(19) United States
(12) Patent Application Publication
(10) Pub. No.: US 2026/0219573 A1
(43) Pub. Date: Jul. 30, 2026

(54) RADIATION-SENSITIVE COMPOSITION, METHOD FOR FORMING PATTERN, AND ONIUM SALT COMPOUND

(71) Applicant: JSR CORPORATION, Tokyo (JP)

(72) Inventors: Ryuichi NEMOTO, Tokyo (JP); Kensuke MIYAO, Tokyo (JP); Satoshi OKAZAKI, Tokyo (JP); Hajime INAMI, Tokyo (JP); Yudai ABE, Tokyo (JP)

(73) Assignee: JSR CORPORATION, Tokyo (JP)

(21) Appl. No.: 19/574,792

(22) Filed: Mar. 23, 2026

Related U.S. Application Data
(63) Continuation-in-part of application No. PCT/JP2024/032863, filed on Sep. 13, 2024.

(30) Foreign Application Priority Data
Sep. 26, 2023 (JP) 2023-162948

(51) Int. Cl. G03F 7/004; C07C 309/23; C07C 381/12; C07D 321/10; C07D 327/04; C07D 333/46; C07D 493/08; G03F 7/00; G03F 7/038; G03F 7/039; G03F 7/20

(52) U.S. Cl. CPC G03F 7/0045; C07C 309/23; C07C 381/12; C07D 321/10; C07D 327/04; C07D 333/46; C07D 493/08; G03F 7/0382; G03F 7/0388; G03F 7/0397; G03F 7/2002; G03F 7/70008

(57) ABSTRACT

A radiation-sensitive composition includes: a first onium salt compound represented by formula (1); at least one second onium salt compound selected from the group consisting of a carboxylic acid salt compound represented by formula (2) and a carboxylic acid intramolecular salt compound represented by formula (3); a polymer including a structural unit which includes an acid-dissociable group; and a solvent. In the formula (1), A is a (1+n)-valent organic group having 1 to 40 carbon atoms; and R^{f1} and R^{f2} are each independently a hydrogen atom, a monovalent organic group, a fluorine atom, or a monovalent fluorinated hydrocarbon group. In the formula (2), R¹ is a monovalent organic group having 2 to 40 carbon atoms. In the formula (3), R² is a single bond or a divalent organic group having 1 to 40 carbon atoms.

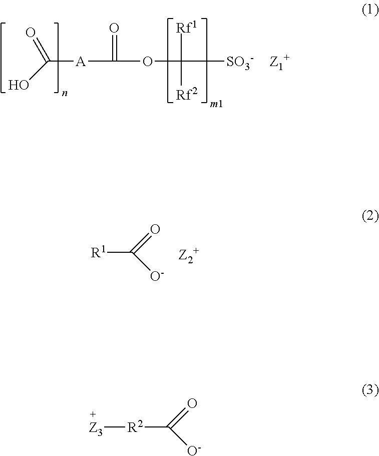

### CROSS-REFERENCE TO RELATED APPLICATIONS

The present application is a continuation-in-part application of International Patent Application No. PCT/JP2024/032863 filed Sep. 13, 2024, which claims priority to Japanese Patent Application No. 2023-162948 filed Sep. 26, 2023. The contents of these applications are incorporated herein by reference in their entirety.

### BACKGROUND OF THE DISCLOSURE

### Technical Field

The present disclosure relates to a radiation-sensitive composition, a method for forming a pattern and an onium salt compound.

### Background Art

A photolithography technology using a resist composition has been used for the fine circuit formation in a semiconductor device. As the representative procedure, for example, a resist pattern is formed on a substrate by generating an acid by irradiating the coating of the resist composition with a radioactive ray through a mask pattern, and then reacting in the presence of the acid as a catalyst to generate the difference of solubility of a resin into an alkaline or organic developer between an exposed part and a non-exposed part.

In the photolithography technique, the micronization of the pattern is promoted by using a short-wavelength radioactive ray such as an ArF excimer laser or by using an immersion exposure method (liquid immersion lithography) in which exposure is performed in a state in which a space between a lens of an exposure apparatus and a resist film is filled with a liquid medium. As a next-generation technology, lithography using shorter wavelength radiation such as electron beams, X-rays and EUV (extreme ultraviolet rays) is also being considered.

To form a finer resist pattern in the formation of a circuit of a semiconductor device by a photolithography technique, various studies have been conducted on a photoacid generator, which is one of main components of a resist composition (see, for example, JP-B2-5083528).

### SUMMARY

According to an aspect of the present disclosure, a radiation-sensitive composition includes:
a first onium salt compound represented by formula (1);
at least one second onium salt compound selected from the group consisting of a carboxylic acid salt compound represented by formula (2) and a carboxylic acid intramolecular salt compound represented by formula (3);
a polymer including a structural unit having an acid-dissociable group; and
a solvent,

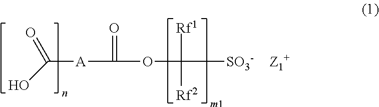

wherein, in the formula (1),
A is a (1+n)-valent organic group having 1 to 40 carbon atoms;
R^{f1} and R^{f2} are each independently a hydrogen atom, a monovalent organic group, a fluorine atom, or a monovalent fluorinated hydrocarbon group; when there are a plurality of R^{f1}s and R^{f2}s, the plurality of R^{f1}s and R^{f2}s are the same as or different from each other;
m1 is an integer of 1 to 8;
n is an integer of 1 to 3; and
Z₁⁺ represents a radiation-sensitive onium cation,

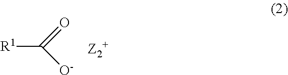

wherein, in the formula (2),
R¹ is a monovalent organic group having 2 to 40 carbon atoms; and
Z₂⁺ represents an organic cation, and

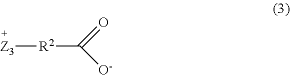

wherein, in the formula (3),
R² is a single bond or a divalent organic group having 1 to 40 carbon atoms; and
Z₃⁺ is a monovalent organic onium cation.

According to another aspect of the present disclosure, a method for forming a pattern, includes:
applying the above-described radiation-sensitive composition directly or indirectly to a substrate to form a resist film;
exposing the resist film to light; and
developing the exposed resist film with a developer.

According to a further aspect of the present disclosure, an onium salt compound is represented by formula (1-a):

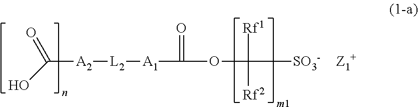

wherein, in the formula (1-a),
A₁ is a divalent organic group;
A₂ is a (1+n)-valent cyclic group;
L₂ is a divalent chain linking group comprising an oxygen atom;
R^{f1} and R^{f2} are each independently a hydrogen atom, a monovalent organic group, a fluorine atom, or a monovalent fluorinated hydrocarbon group; when there are a plurality of R^{f1}s and R^{f2}s, the plurality of R^{f1}s and R^{f2}s are the same as or different from each other;
m1 is an integer of 1 to 8;
n is an integer of 1 to 3; and
Z₁⁺ represents a radiation-sensitive onium cation.

### DESCRIPTION OF THE EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed.

Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

The resist composition is required to have various resist performances such as sensitivity, line width roughness (LWR) performance, which indicates variations in a line width, and a line width of a resist pattern, pattern rectangularity, which indicates rectangularity of a sectional shape of a resist pattern, critical dimension uniformity (CDU) performance, which is an index of uniformity of a hole diameter, pattern circularity, mask error enhancement factor (MEEF), and exposure margin (DOF).

Since the radiation-sensitive composition of the present disclosure contains the first onium salt compound and the second onium salt compound, a resist film exhibiting excellent sensitivity, LWR performance, pattern rectangularity, CDU performance, pattern circularity, MEEF and DOF can be formed. The reason for this is not bound by any theory, but can be expected as follows.

The carboxy group of the anion of the first onium salt compound interacts with the polymer in the composition, so that the diffusion length of the generated acid can be appropriately shortened. Since the anion of the first onium salt compound has a carboxy group, solubility in a developer is greatly improved as compared with an onium salt compound having a polar group such as a hydroxyl group, a lactone structure, and a sultone structure that has been developed so far, and LWR performance and CDU performance can be improved. Since the second onium salt compound has a carboxylic acid anion structure, the quenching ability is high, the proton capture of the acid generated from the first onium salt compound or the like can be efficiently promoted, and the acid diffusion controlling ability in the non-exposed part of the first onium salt compound is improved. Since the second onium salt compound has a carboxylic acid anion structure and the first onium salt compound has a carboxy group, the compatibility of the two types of onium salts is improved, and in particular, aggregation of the first onium salt compound can be eliminated to improve dispersibility. As a result, excellent pattern rectangularity and pattern circularity can be exhibited. Furthermore, it is presumed that given resist properties can also be exhibited.

In the method for forming a pattern of the present disclosure, a high-quality resist pattern can be efficiently formed because of the use of the radiation-sensitive composition capable of forming a resist film excellent in sensitivity, LWR performance, pattern rectangularity, CDU performance, pattern circularity, MEEF and DOF.

Since the radiation-sensitive acid generator of the present disclosure contains the onium salt compound, good sensitivity, LWR performance, pattern rectangularity, CDU performance, pattern circularity, MEEF and DOF can be imparted to a resist film obtained.

Hereinbelow, embodiments of the present disclosure will be described in detail, but the present disclosure is not limited to these embodiments. Combinations of suitable embodiments are also preferable.

### <<Radiation-Sensitive Composition>>

The radiation-sensitive composition (hereinafter also simply referred to as “composition”) according to the present embodiment includes the first onium salt compound, the second onium salt compound, a polymer (A) containing a structural unit (I) having an acid-dissociable group, and a solvent (E). The composition may further contain other optional components as long as the effects of the present invention are not impaired.

### <Polymer (A)>

The polymer (A) is an aggregate of polymers having a structural unit (hereinafter, also referred to as “structural unit (I)”) containing an acid-dissociable group (hereinafter, this aggregate is also referred to as “base polymer”). The “acid-dissociable group” refers to a group that substitutes for a hydrogen atom of a carboxy group, a phenolic hydroxyl group, an alcoholic hydroxyl group, a sulfo group, or the like, and is dissociated by the action of an acid. An acid generated from the first onium salt compound through exposure to light dissociates the acid-dissociable group in the structural unit (I) to generate a carboxy group or the like. As a result, a difference in solubility into a developer arises between the exposed part and the non-exposed part of a resist film, making it possible to achieve pattern formation.

The polymer (A) may have another structural unit other than the structural unit (I). For example, the polymer (A) may have the following structural units (II) to (IV) and structural unit containing an alicyclic structure. Each of the structural units will be described below.

### [Structural Unit (I)]

The structural unit (I) is not particularly limited as long as it contains an acid-dissociable group. Examples of such a structural unit (I) include a structural unit having a tertiary alkyl ester moiety, a structural unit having a structure obtained by substituting the hydrogen atom of a phenolic hydroxyl group with a tertiary alkyl group, and a structural unit having an acetal bond. From the viewpoint of improving the pattern-forming performance of the radiation-sensitive composition, a structural unit represented by the formula (4) (hereinafter also referred to as a “structural unit (I-1)”) is preferred.

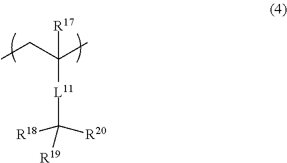

In the formula (4), R¹⁷ is a hydrogen atom, a fluorine atom, a methyl group, or a trifluoromethyl group, R¹⁸ is a substituted or unsubstituted monovalent hydrocarbon group having 1 to 20 carbon atoms, R¹⁹ and R²⁰ are each independently a substituted or unsubstituted monovalent chain hydrocarbon group having 1 to 20 carbon atoms or a substituted or unsubstituted monovalent alicyclic hydrocarbon group having 3 to 20 carbon atoms, or R¹⁹ and R²⁰ taken together represent a divalent alicyclic group having 3 to 20 carbon atoms together with the carbon atom to which R¹⁹ and R²⁰ are bonded, L₁₁ represents *—COO—, *-L^{11a}COO—, or *—COOL^{11a}COO—; L^{11a} is a substituted or unsubstituted alkanediyl group or arenediyl group, and * is a bond to a carbon atom to which R¹⁷ is bonded.

From the viewpoint of copolymerizability of a monomer that will give the structural unit (1-1), R¹⁷ is preferably a hydrogen atom or a methyl group, more preferably a methyl group.

Examples of the monovalent hydrocarbon group having 1 to 20 carbon atoms represented by R¹⁸ include a chain hydrocarbon group having 1 to 20 carbon atoms, a monovalent alicyclic hydrocarbon group having 3 to 20 carbon atoms, and a monovalent aromatic hydrocarbon group having 6 to 20 carbon atoms.

Examples of the monovalent chain hydrocarbon group having 1 to 20 carbon atoms represented by R¹⁸ to R²⁰ include a monovalent linear or branched chain saturated hydrocarbon group having 1 to 20 carbon atoms and a monovalent linear or branched chain unsaturated hydrocarbon group having 2 to 20 carbon atoms. Examples of the monovalent linear or branched chain saturated hydrocarbon group having 1 to 20 carbon atoms include alkyl groups such as a methyl group, an ethyl group, a n-propyl group, an i-propyl group, a n-butyl group, a 2-methylpropyl group, a 1-methylpropyl group, a t-butyl group, a n-pentyl group, an isopentyl group, and a neopentyl group. Examples of the monovalent linear or branched chain unsaturated hydrocarbon group having 2 to 20 carbon atoms include alkenyl groups such as an ethenyl group, a propenyl group, and a butenyl group; and alkynyl groups such as an ethynyl group, a propynyl group, and a butynyl group.

Examples of the monovalent alicyclic hydrocarbon group having 3 to 20 carbon atoms represented by R¹⁸ to R²⁰ include monovalent monocyclic or polycyclic saturated hydrocarbon groups and monocyclic or polycyclic unsaturated hydrocarbon groups. Examples of the monocyclic saturated hydrocarbon groups include cycloalkyl groups such as a cyclopentyl group, a cyclohexyl group, a cycloheptyl group, and a cyclooctyl group. Examples of the polycyclic saturated hydrocarbon group include bridged alicyclic hydrocarbon groups such as a norbornyl group, an adamantyl group, a tricyclodecyl group, and a tetracyclododecyl group. Examples of the monocyclic unsaturated hydrocarbon group include monocyclic cycloalkenyl groups such as a cyclopropenyl group, a cyclobutenyl group, a cyclopentenyl group, and a cyclohexenyl group. Examples of the polycyclic unsaturated hydrocarbon group include polycyclic cycloalkenyl groups such as a norbornenyl group, a tricyclodecenyl group, and a tetracyclododecenyl group. The bridged alicyclic hydrocarbon group refers to a polycyclic alicyclic hydrocarbon group in which two carbon atoms that constitute an alicyclic ring and are not adjacent to each other are bonded by a linking group containing one or more carbon atoms.

Examples of the monovalent aromatic hydrocarbon group having 6 to 20 carbon atoms represented by R¹⁸ include: aryl groups such as a phenyl group, a tolyl group, a xylyl group, a naphthyl group, and an anthryl group; and aralkyl groups such as a benzyl group, a phenethyl group, and a naphthylmethyl group.

R¹⁸ is preferably a linear or branched chain saturated hydrocarbon group having 1 to 20 carbon atoms or a monovalent alicyclic hydrocarbon group having 3 to 20 carbon atoms.

The divalent alicyclic group having 3 to 20 carbon atoms formed by the chain hydrocarbon group or the alicyclic hydrocarbon group represented by R¹⁹ and R²⁰ taken together with the carbon atom to which R¹⁹ and R²⁰ are bonded is not particularly limited as long as it is a group obtained by removing one hydrogen atoms from the same carbon atom constituting a carbon ring of a monocyclic or polycyclic alicyclic hydrocarbon having 3 to 20 carbon atoms. The divalent alicyclic group having 3 to 20 carbon atoms may either be a monocyclic hydrocarbon group or a polycyclic hydrocarbon group. The polycyclic hydrocarbon group may either be a bridged alicyclic hydrocarbon group or a condensed alicyclic hydrocarbon group and may either be a saturated hydrocarbon group or an unsaturated hydrocarbon group. It is to be noted that the condensed alicyclic hydrocarbon group refers to a polycyclic alicyclic hydrocarbon group in which two or more alicyclic rings share their sides (bond between two adjacent carbon atoms).

When the monocyclic alicyclic hydrocarbon group is a saturated hydrocarbon group, preferred examples thereof include a cyclopentanediyl group, a cyclohexanediyl group, a cycloheptanediyl group, and a cyclooctanediyl group. When the monocyclic alicyclic hydrocarbon group is an unsaturated hydrocarbon group, preferred examples thereof include a cyclopentenediyl group, a cyclohexenediyl group, a cycloheptenediyl group, a cyclooctenediyl group, and a cyclodecenediyl group. The polycyclic alicyclic hydrocarbon group is preferably a bridged alicyclic saturated hydrocarbon group, and preferred examples thereof include a bicyclo[2.2.1]heptane-2,2-diyl group (norbornane-2,2-diyl group), a bicyclo[2.2.2]octane-2,2-diyl group, and a tricyclo[3.3.1.1^{3,7}]decane-diyl group (adamantane-diyl group).

As R¹⁹ and R²⁰, a monovalent chain hydrocarbon group having 1 to 10 carbon atoms or a divalent alicyclic hydrocarbon group having 3 to 20 carbon atoms composed of R¹⁹ and R²⁰ combined with each other together with a carbon atom to which R¹⁹ and R²⁰ are bonded is preferable, a monovalent linear hydrocarbon group having 1 to 10 carbon atoms or a divalent alicyclic hydrocarbon group having 5 to 10 carbon atoms is more preferable, and a methyl group, an ethyl group, a cyclopentanediyl group, or an adamantanediyl group is still more preferable.

When R¹⁸ to R²⁰ have substituents, examples of the substituent (T) include a halogen atom such as a fluorine atom, a chlorine atom, a bromine atom, and an iodine atom; a hydroxy group; a carboxy group; a cyano group; a nitro group; an alkyl group, an alkoxy group, an alkoxycarbonyl group, an alkoxycarbonyloxy group, an acyl group, an acyloxy group, an aryloxy group, or groups obtained by substituting a hydrogen atom on these groups with a halogen atom; and an oxo group (═O).

Examples of the alkanediyl group represented by L^{11a} include alkanediyl groups having 1 to 10 carbon atoms such as a methylene group, an ethanediyl group, a 1,3-propanediyl group, and a 2,2-propanediyl group. L^{11a} is preferably a methylene group or an ethanediyl group.

Examples of the arenediyl group represented by L^{11a} include divalent aromatic hydrocarbon groups having 6 to 20 carbon atoms such as a benzenediyl group and a naphthalenediyl group. As Lila, a benzenediyl group is preferable.

Examples of the substituent that can be possessed by the arenediyl group represented by L^{11a} include a halogen atom, a hydroxy group, a carboxy group, a cyano group, a nitro group, an alkyl group, a fluorinated alkyl group, an alkoxycarbonyloxy group, an acyl group, an acyloxy group, and an alkoxy group.

Examples of the structural unit (I) include structural units represented by the formulas (4-1) to (4-15) (hereinafter also referred to as “structural units (I-1) to (I-15)”).

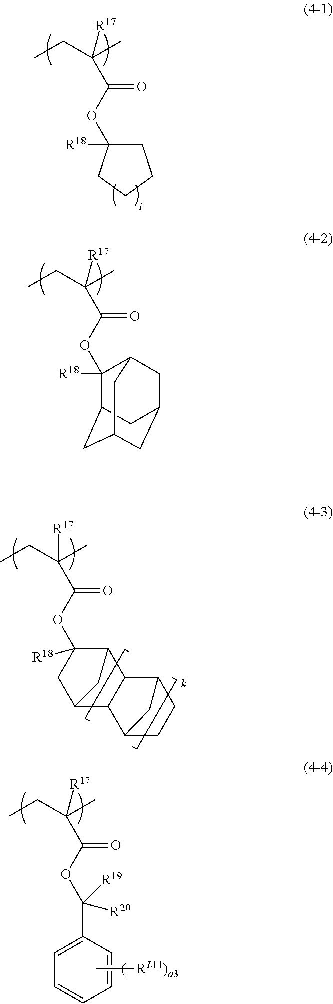

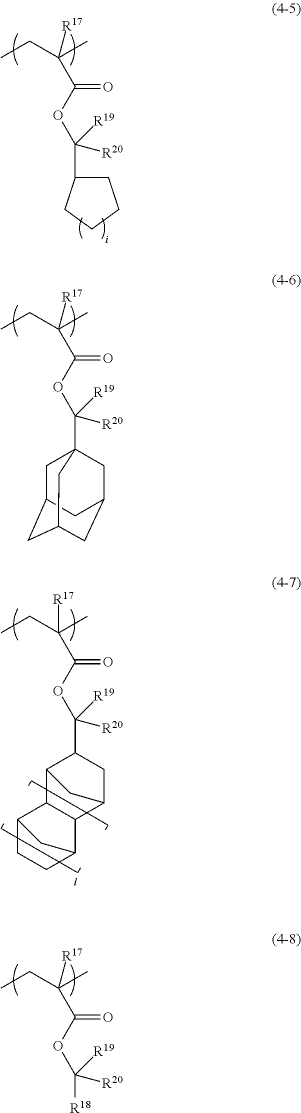

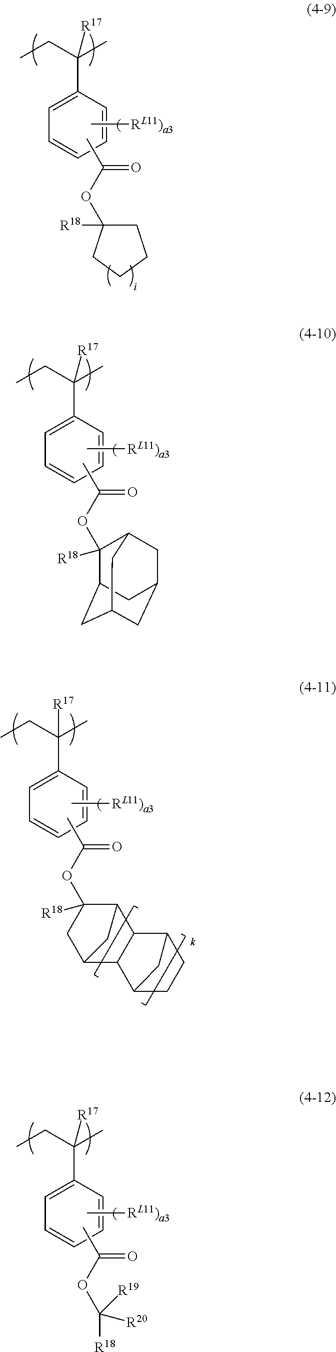

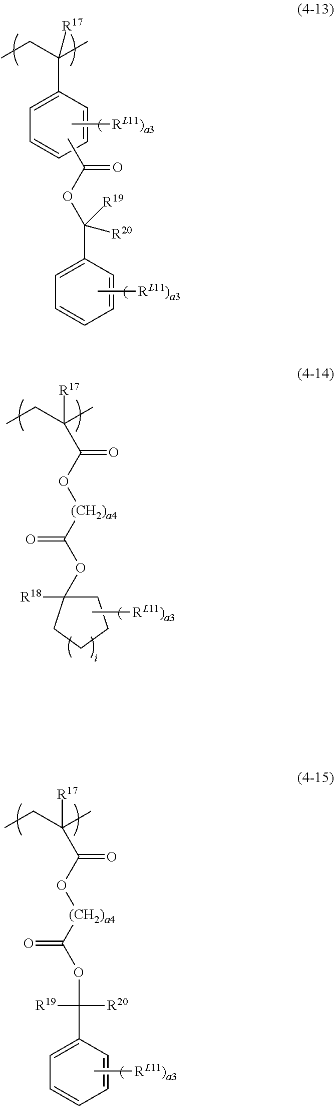

In the formulas (4-1) to (4-15), R¹⁷ to R²⁰ have the same meaning as in the formula (4), R^{L1l} is a halogen atom, a hydroxy group, a carboxy group, a cyano group, a nitro group, an alkyl group, a fluorinated alkyl group, an alkoxycarbonyl-oxy group, an acyl group, an acyloxy group, or an alkoxy group, i and j are each independently an integer of 1 to 4, k and l are each 0 or 1, a3 is each independently an integer of 0 to 3, when a3 is 2 or more, a plurality of R^{L11}'s may be the same or different, and a4 is an integer of 1 to 3.

The i and j are preferably 1, and R¹⁸ is preferably a methyl group, an ethyl group, an isopropyl group, a t-butyl group, a cyclopentyl group or an adamantyl group. R¹⁹ and R²⁰ are each preferably a methyl group, or an ethyl group.

The polymer may further contain, as a structural unit (I), a structural unit represented by the formula (1f) to (2f).

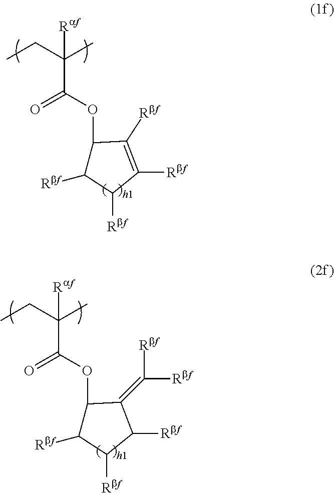

In the above formulas (1f) to (2f), Raf is each independently a hydrogen atom, a fluorine atom, a methyl group or a trifluoromethyl group, Rβf is each independently a hydrogen atom or a chain alkyl group having 1 to 5 carbon atoms, and h1 is an integer of 1 to 4.

R^{βf} is preferably a hydrogen atom, a methyl group or an ethyl group. h1 is preferably 1 or 2.

Specific examples of the structural unit (I) include, but are not limited to, structural units represented by the formulas.

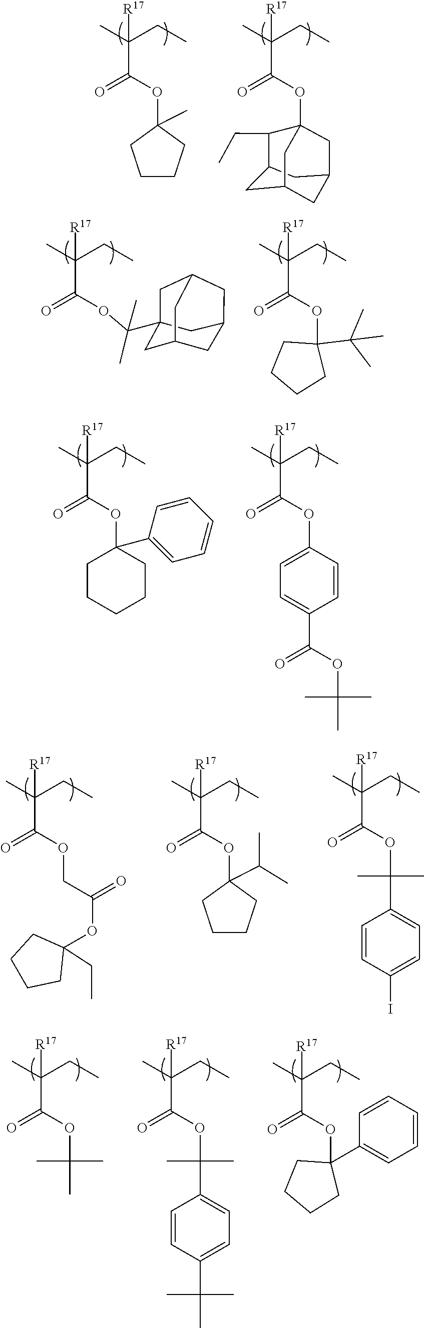

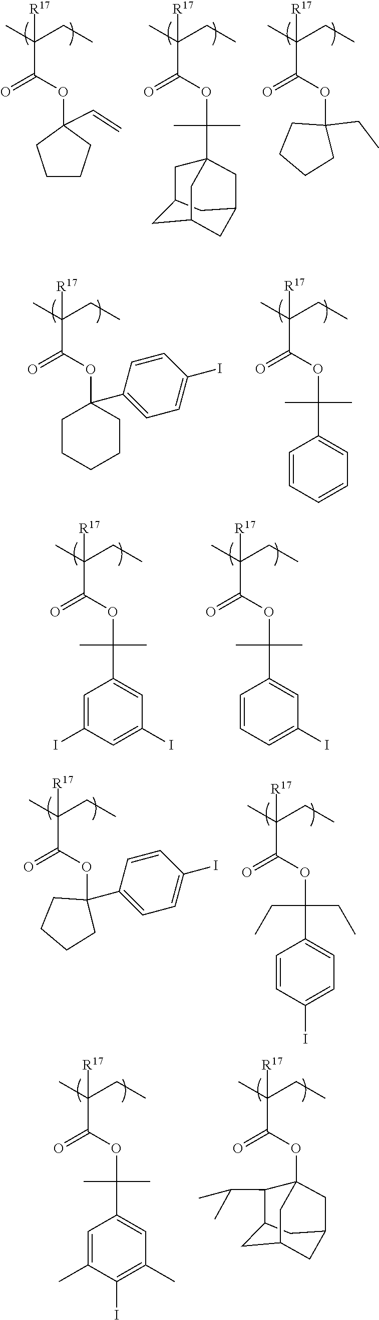

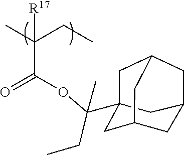

In the formula, R¹⁷ has the same meaning as in the formula (4).

The base polymer may contain one type or a combination of two or more types of the structural units (I).

The lower limit of the content of the structural unit (I) (a total content when a plurality of types are contained) is preferably 5 mol %, more preferably 7 mol %, and still more preferably 10 mol % based on all structural units constituting the base polymer. The upper limit of the content is preferably 80 molo, more preferably 70 mol %, and still more preferably 60 mol %. When the content of the structural unit (I) is set to fall within the above range, the pattern-forming performance of the radiation-sensitive composition can further be improved.

### [Structural Unit (II)]

The base polymer optionally has another structural unit in addition to the structural units (I). Another structural unit includes a structural unit (II) containing at least one selected from the group consisting of a lactone structure, a cyclic carbonate structure, and a sultone structure. The solubility of the base polymer into a developer can be adjusted by further introducing the structural unit (II). As a result, the radiation-sensitive composition can provide improved lithography properties such as the resolution. The adhesion between a resist pattern formed from the base polymer and a substrate can also be improved.

Examples of the structural unit (II) include structural units represented by the formulae (T-1) to (T-11).

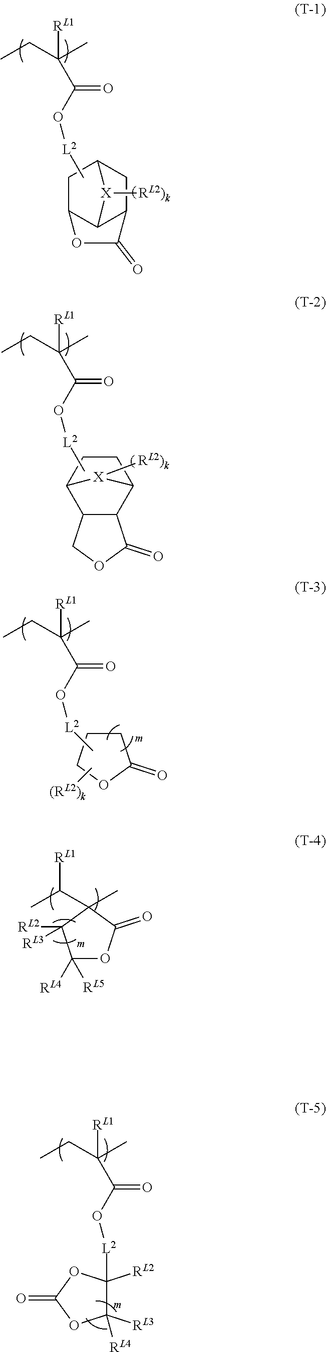

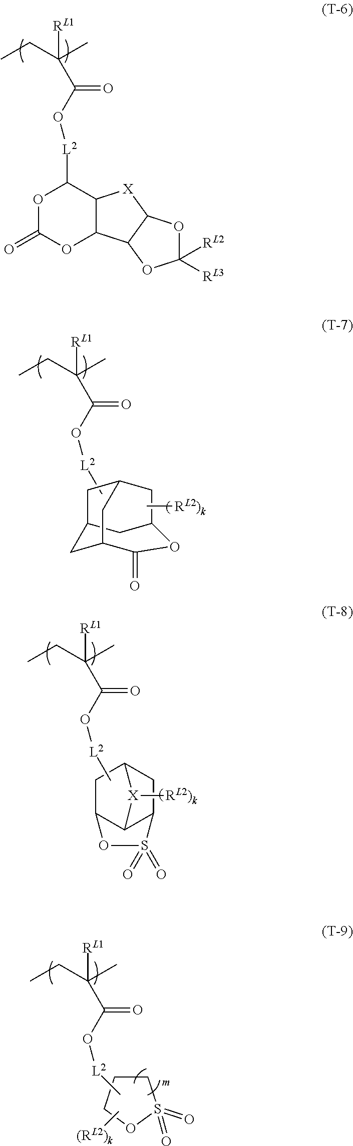

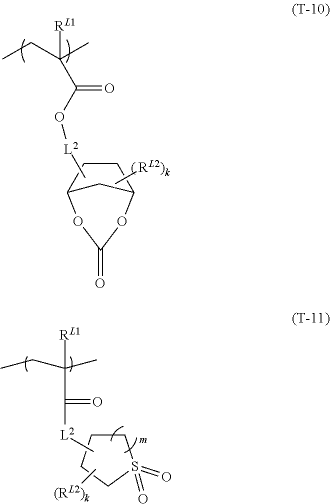

In the formulae, R^{L1} is a hydrogen atom, a fluorine atom, a methyl group, or a trifluoromethyl group; R¹² to R¹⁵ are each independently a hydrogen atom, an alkyl group having 1 to 4 carbon atoms, a cyano group, a trifluoromethyl group, a methoxy group, a methoxycarbonyl group, a hydroxy group, a hydroxymethyl group, or a dimethylamino group; R^{L4} and R^{L5} may be a divalent alicyclic hydrocarbon group having 3 to 8 carbon atoms, which is obtained by combining R^{L4} and R^{L5} with the carbon atom to which they are bound. L² is a single bond, or a divalent linking group; X is an oxygen atom or a methylene group; k is an integer of 0 to 3; and m is an integer of 1 to 3.

Example of the divalent alicyclic hydrocarbon group having 3 to 8 carbon atoms, which is composed of a combination of R^{L4} and R^{L5} with the carbon atom to which they are bound, includes the divalent alicyclic group having 3 to 8 carbon atoms in the divalent alicyclic hydrocarbon group having 3 to 20 carbon atoms, which is obtained by combining R¹⁹ and R²⁰ in the formula (4) with the carbon atom to which they are bound. One or more hydrogen atoms on the alicyclic hydrocarbon group may be substituted with a hydroxy group.

Examples of the divalent linking group represented by L² as described above include a divalent linear or branched chain hydrocarbon group having 1 to 10 carbon atoms; a divalent alicyclic hydrocarbon group having 4 to 12 carbon atoms; and a group composed of one or more of the hydrocarbon group thereof and at least one group of —CO—, —O—, —NH— and —S—.

As the linear or branched chain divalent hydrocarbon group having 1 to 10 carbon atoms in L², a group obtained by removing one hydrogen atom from the chain hydrocarbon group having 1 to 20 carbon atoms represented by R¹⁸ of the formula (4) can be suitably employed.

As the divalent alicyclic hydrocarbon group having 4 to 12 carbon atoms in L₂, a group obtained by removing one hydrogen atom from the monovalent alicyclic hydrocarbon group having 3 to 20 carbon atoms represented by R¹⁸ of the formula (4) can be suitably employed.

Among them, the structural unit (II) is preferably a group having a lactone structure, more preferably a group having a norbornane lactone structure, and further preferably a group derived from a norbornane lactone-yl (meth)acrylate.

The base polymer may contain one type or a combination of two or more types of the structural units (II).

When the base polymer has the structural unit (II), the lower limit of the content of the structural unit (II) (a total content when a plurality of types are contained) is preferably 5 mol %, more preferably 10 mol %, and still more preferably 15 mol % based on all structural units constituting the base polymer. The upper limit of the content is preferably 80 mol %, more preferably 70 mol %, and still more preferably 60 mol %. By adjusting the content of the structural unit (II) within the ranges, the radiation-sensitive composition can provide improved lithography properties such as the resolution. The adhesion between the formed resist pattern and the substrate can also be improved.

### [Structural Unit (III)]

The base polymer may include a structural unit (III) containing a polar group (excluding those corresponding to the structural unit (II)). When the base polymer further has a structural unit (III), solubility in the developer can be adjusted. As a result, lithographic performance such as resolution of the radiation-sensitive composition can be improved. Examples of the polar group include a hydroxy group, a carboxy group, a cyano group, a nitro group, and a sulfonamide group. Among them, a hydroxy group and a carboxy group are preferable, and a hydroxy group is more preferable.

Examples of the structural unit (III) include structural units represented by the formulas.

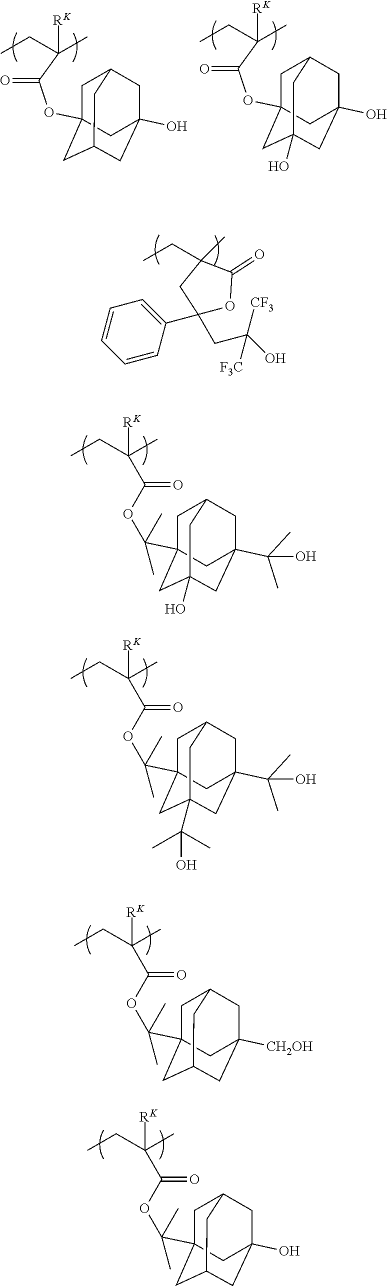

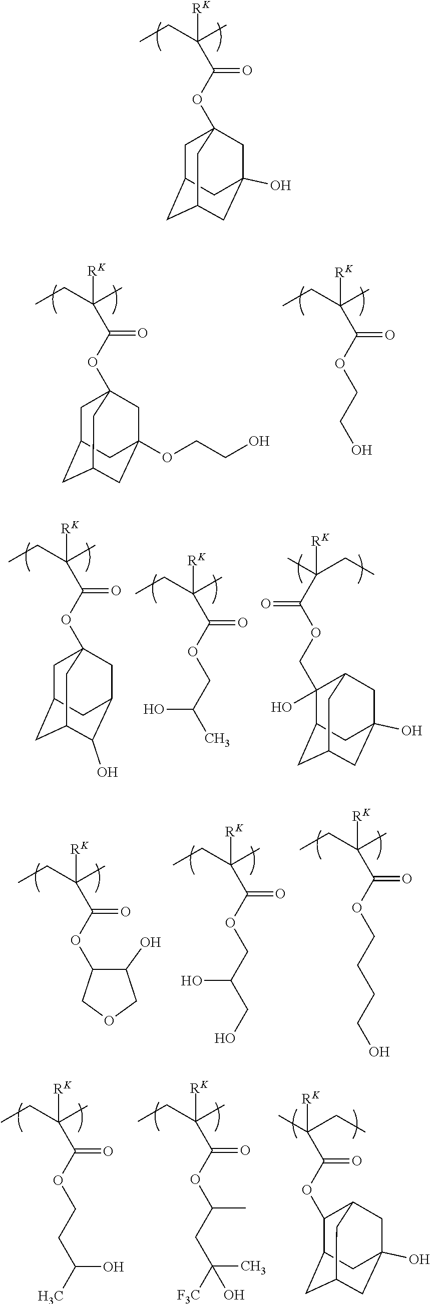

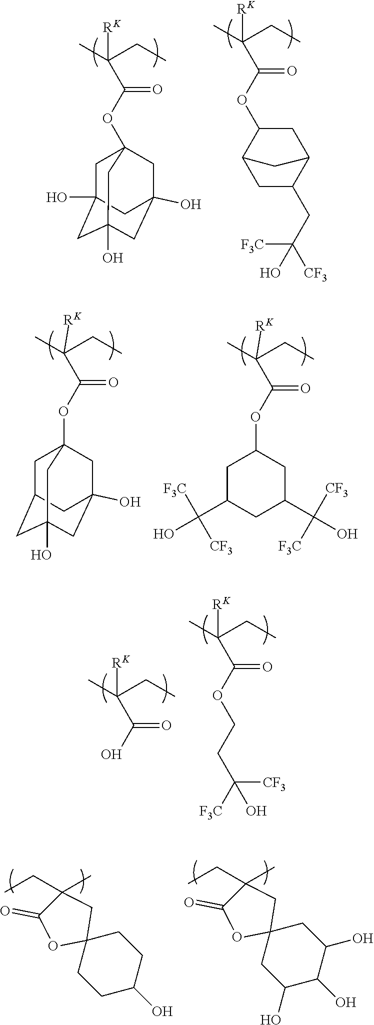

In the formulas, R^{K} is a hydrogen atom, a fluorine atom, a methyl group, or a trifluoromethyl group.

When the base polymer has the structural unit (III), the lower limit of the content of the structural unit (III) is preferably 2 mol %, more preferably 5 mol %, and still more preferably 8 mol % based on all structural units constituting the base polymer. The upper limit of the content is preferably 80 molo, more preferably 70 mol %, and still more preferably 65 mol %. When the content of the structural unit (III) is set to fall within the above range, the radiation-sensitive composition can provide further improved lithography properties such as the resolution.

### [Structural Unit (IV)]

The base polymer optionally has, as another structural unit, a structural unit (IV) having a phenolic hydroxyl group. When the polymer includes the structural unit (IV), the solubility into a developer can be more appropriately adjusted, and as a result, the sensitivity and the like of the radiation-sensitive composition can be further improved. When KrF excimer laser light, EUV, electron beam, or the like is used as radiation to be applied in an exposure step in a resist pattern formation method, the structural unit (IV) contributes to improvement in etching resistance and improvement in difference in solubility in a developer between an exposed part and a non-exposed part (dissolution contrast). In particular, the structural unit (IV) can be suitably applied to pattern formation using exposure with a radioactive ray having a wavelength of 50 nm or less, such as an electron beam or EUV. The Structural unit (IV) is preferably represented by the formula (5).

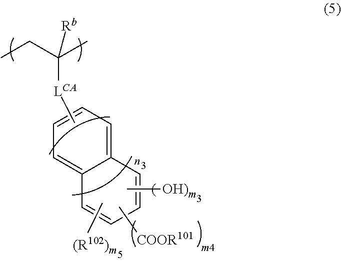

In the formula (5),
R^{b} is a hydrogen atom, a fluorine atom, a methyl group, or a trifluoromethyl group;
L^{CA} is a single bond, —COO—*, or —O—; * is a bond on the aromatic ring side;
R¹⁰¹ is an acid-dissociable group;
R¹⁰² is a halogen atom, a cyano group, a nitro group, an alkyl group, an alkoxycarbonyl group, an acyl group, or an acyloxy group; when there are a plurality of R¹⁰²s, the plurality of R¹⁰²s are the same as or different from each other; and
n₃ is an integer of 0 to 2, m₃ is an integer of 1 to 8, and me and my are each independently an integer of 0 to 8, provided that 1≤m₃+m₄+m₅≤2n₃+5 is satisfied.

From the viewpoint of copolymerizability of a monomer that will give the structural unit (IV), Rb is preferably a hydrogen atom or a methyl group.

As the L^{CA}, a single bond or —COO—* is preferred.

The acid-dissociable group represented by R¹⁰¹ is not particularly limited, and examples thereof include a structure in which R¹⁰¹ is bonded to —COO— to form a tertiary alkyl ester moiety, a structure in which R¹⁰¹ is bonded to —COO— to form a secondary unsaturated alkyl ester moiety having a double bond between the β-position carbon and the γ-position carbon of the terminal oxygen atom of —COO—, and a structure in which R¹⁰¹ is bonded to —COO— to form an acetal bond.

Examples of the halogen atom as R¹⁰² include a fluorine atom, a chlorine atom, a bromine atom, and an iodine atom, and a fluorine atom and an iodine atom are preferable. Examples of the alkyl group as R¹⁰² include linear or branched chain alkyl groups having 1 to 8 carbon atoms such as a methyl group, an ethyl group, and a propyl group. Examples of the alkoxycarbonyl group as R¹⁰² include a methoxycarbonyl group, an ethoxycarbonyl group, and an n-butoxycarbonyl group. Examples of the acyl group include aliphatic or aromatic acyl groups having 2 to 12 carbon atoms such as an acetyl group, a propionyl group, a benzoyl group, and an acryloyl group. Examples of the acyloxy group include aliphatic or aromatic acyloxy groups having 2 to 12 carbon atoms such as an acetyloxy group, a propionyloxy group, a benzoyloxy group, and an acryloyloxy group.

The n₃ is more preferably 0 or 1, and still more preferably 0.

The m₃ is preferably an integer of 1 to 3, and more preferably 1 or 2.

The m₄ is preferably an integer of 0 to 3, and more preferably an integer of 0 to 2.

The m₅ is preferably an integer of 0 to 3, and more preferably an integer of 0 to 2.

Preferred examples of the structural unit (IV) include the structural units represented by the formulas (5-1) to (5-32) (hereinafter, also referred to as “structural unit (IV-1) to (IV-32)”).

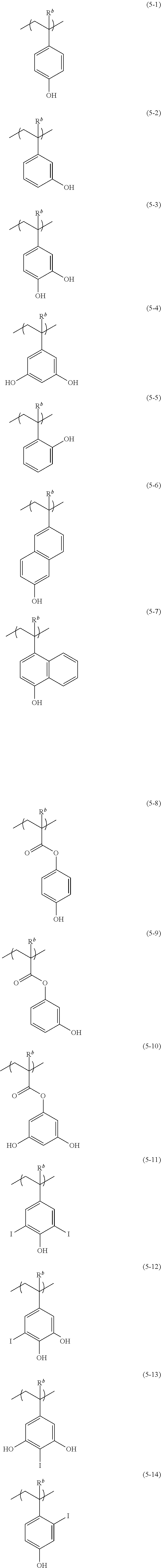

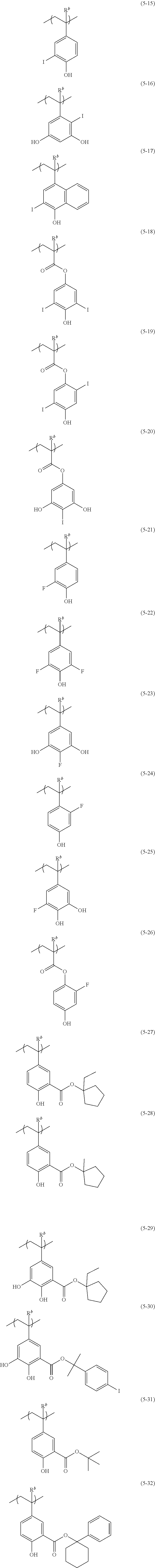

In the above formulas (5-1) to (5-32), Rb is the same as in the formula (5).

When the base polymer has the structural unit (IV) containing a polar group, the lower limit of the content of the structural unit (IV) (a total content when a plurality of types of the structural unit (IV) are contained) is preferably 10 mol %, more preferably 20 mol %, and still more preferably 30 mol % based on all structural units constituting the polymer. The upper limit of the content is preferably 70 mol %, more preferably 60 mol %, and still more preferably 50 mol %. When the content ratio of the structural unit (IV) is adjusted to within the above range, the sensitivity and the development contrast of the radiation-sensitive composition can be further improved.

In the case of polymerizing a monomer having a phenolic hydroxy group such as hydroxystyrene, it is preferable to polymerize the monomer with the phenolic hydroxy group protected by a protective group such as an alkali-dissociable group (for example, an acyl group), and then perform deprotection by hydrolysis to obtain a structural unit (IV). The polymerization may be conducted without protecting a phenolic hydroxy group of hydroxystyrene or the like.

### [Other Structural Unit]

The base polymer may contain, as a structural unit other than the structural units listed above, a structural unit represented by the formula (6) and containing an alicyclic structure.

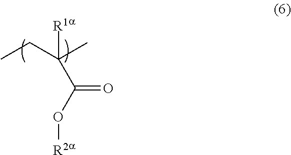

In the formula (6), R^{1α} represents a hydrogen atom, a fluorine atom, a methyl group, or a trifluoromethyl group, and R^{2α} represents a monovalent alicyclic hydrocarbon group having 3 to 20 carbon atoms.

In the formula (6), as the monovalent alicyclic hydrocarbon group having 3 to 20 carbon atoms represented by R^{2α}, the monovalent alicyclic hydrocarbon groups having 3 to 20 carbon atoms represented by R¹⁸ in the formula (4) can be suitably employed.

When the base polymer contains the structural unit having an alicyclic structure, the lower limit of the content of the structural unit having an alicyclic structure is preferably 2 mol %, more preferably 5 mol %, and still more preferably 8 mol % based on all structural units constituting the base polymer. The upper limit of the content is preferably 30 mol %, more preferably 20 mol %, and still more preferably 15 mol %.

### (Synthesis Method of Base Polymer)

For example, the base polymer can be synthesized by performing a polymerization reaction of each monomer for providing each structural unit with a radical polymerization initiator or the like in a suitable solvent.

Examples of the radical polymerization initiator include an azo-based radical initiator, including azobisisobutyronitrile (AIBN), 2,2′-azobis(4-methoxy-2,4-dimethylvaleronitrile), 2,2′-azobis(2-cyclopropylpropanenitrile), 2,2′-azobis(2,4-dimethylvaleronitrile), and dimethyl 2,2′-azobisisobutyrate; and peroxide-based radical initiator, including benzoyl peroxide, t-butyl hydroperoxide, and cumene hydroperoxide. Among them, AIBN or dimethyl 2,2′-azobisisobutyrate is preferred, and AIBN is more preferred. The radical initiator may be used alone, or two or more radical initiators may be used in combination.

Examples of the solvent used for the polymerization reaction include
alkanes including n-pentane, n-hexane, n-heptane, n-octane, n-nonane, and n-decane;
cycloalkanes including cyclohexane, cycloheptane, cyclooctane, decalin, and norbornane;
aromatic hydrocarbons including benzene, toluene, xylene, ethylbenzene, and cumene;
halogenated hydrocarbons including chlorobutanes, bromohexanes, dichloroethanes, hexamethylenedibromide, and chlorobenzenes;
saturated carboxylate esters, including ethyl acetate, n-butyl acetate, i-butyl acetate, and methyl propionate;
a partially etherized polyhydric alcohol acetate-based solvent, including diethylene glycol mono-n-butyl ether acetate, propylene glycol monomethyl ether acetate, and dipropylene glycol monomethyl ether acetate;
ketones including acetone, 2-butanone, 4-methyl-2-pentanone, and 2-heptanone;
ethers including tetrahydrofuran, dimethoxyethanes, diethoxyethanes and 1,4-dioxane;
alcohols including methanol, ethanol, 1-propanol, 2-propanol, 1-methoxy-2-propanol and 4-methyl-2-pentanol; and
a lactones, including γ-butyrolactone. The solvent used for the polymerization reaction may be used alone, or two or more solvents may be used in combination.

The reaction temperature of the polymerization reaction is typically from 40° C. to 150° C., and preferably from 50° C. to 120° C. The reaction time is typically from 1 hour to 48 hours, and preferably from 1 hour to 24 hours.

The molecular weight of the base polymer is not particularly limited, and the lower limit of the weight-average molecular weight (Mw) equivalent to polystyrene determined by gel permeation chromatography (GPC) is preferably 2,000, more preferably 3,000, still more preferably 4,000, and particularly preferably 4,500. The upper limit of the Mw is preferably 30,000, more preferably 20,000, still more preferably 12,000, and particularly preferably 10,000. By setting the Mw of the base polymer within the above range, it is possible to impart good heat resistance and developability to the resulting resist film.

For the base polymer, the ratio of Mw to the number average molecular weight (Mn) as determined by GPC relative to standard polystyrene (Mw/Mn) is typically 1 or more and 5 or less, preferably 1 or more and 3 or less, and more preferably 1 or more and 2 or less.

The Mw and Mn of the polymer in the specification are amounts measured by using Gel Permeation Chromatography (GPC) with the condition as described below.
GPC column: two G2000HXL, one G3000HXL, and one G4000HXL (all manufactured from Tosoh Corporation)
Column temperature: 40° C.
Eluting solvent: tetrahydrofuran
Flow rate: 1.0 mL/min
Sample concentration: 1.0% by mass
Sample injection amount: 100 μL
Detector: Differential Refractometer
Reference material: monodisperse polystyrene

The content of the base polymer is preferably 60% by mass or more, more preferably 65% by mass or more, and still more preferably 70% by mass or more based on the total solid content of the radiation-sensitive composition.

### (First Onium Salt Compound)

The first onium salt compound is represented by the formula (1).

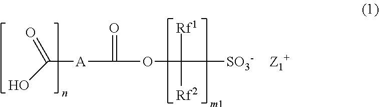

wherein, in the formula (1),
A is a (1+n)-valent organic group having 1 to 40 carbon atoms;
R^{f1} and R^{f2} are each independently a hydrogen atom, a monovalent organic group, a fluorine atom, or a monovalent fluorinated hydrocarbon group; when there are a plurality of R^{f1}s and R^{f2}s, the plurality of R^{f1}s and R^{f2}s are the same as or different from each other;
m1 is an integer of 1 to 8;
n is an integer of 1 to 3; and
Z₁⁺ represents a radiation-sensitive onium cation.

The first onium salt compound has a function in which the acid generated through exposure to light dissociates the acid-dissociable group of the polymer to generate a carboxy group or the like (that is, functions as a radiation-sensitive acid generator). The first onium salt compound as a radiation-sensitive acid generator will be described below.

The (1+n)-valent organic group having 1 to 40 carbon atoms represented by A is not particularly limited, and may include a chain structure, a cyclic structure (hereinafter, a first cyclic structure), or a combination thereof, but preferably includes the first cyclic structure from the viewpoint of appropriately controlling the acid diffusion length. The carboxy group is preferably directly bonded to the first cyclic structure. Note that, in the present specification, the “organic group” is a group containing at least one carbon atom.

Examples of the chain structure include a chain hydrocarbon group having 1 to 40 carbon atoms, which may be saturated, unsaturated, linear, or branched. The first cyclic structure is not particularly limited as long as it has a cyclic structure, and may be any of an alicyclic structure, an aromatic ring structure, and a heterocyclic structure, may be either a monocyclic structure or a polycyclic structure, and may be either saturated or unsaturated. Examples thereof include a group obtained by substituting a part or all of hydrogen atoms contained in a group having a chain structure or a group having a first cyclic structure by a substituent and a group containing, in a carbon-carbon bond (including both between two adjacent carbons and between two non-adjacent carbons) of such a group, a divalent heteroatom-containing group.

As the alicyclic structure, a ring structure corresponding to the monovalent alicyclic hydrocarbon group having 3 to 20 carbon atoms represented by R¹⁸ to R²⁰ of the formula (4) can be employed.

As the aromatic ring structure, a ring structure corresponding to the monovalent aromatic hydrocarbon group having 6 to 20 carbon atoms represented by R¹⁸ of the formula (4) can be employed.

Examples of the heterocyclic structure include an aromatic heterocyclic structure and an alicyclic heterocyclic structure.

Examples of the aromatic heterocyclic structure include: oxygen atom-containing aromatic heterocyclic structures such as furan, pyran, benzofuran, and benzopyran;
nitrogen atom-containing aromatic heterocyclic structures such as pyrrole, imidazole, pyridine, pyrimidine, pyrazine, indole, quinoline, and isoquinoline;
sulfur atom-containing aromatic heterocyclic structures such as thiophene; and
aromatic heterocyclic structures containing a plurality of hetero atoms such as thiazole, benzothiazole, thiazine, and oxazine.

Examples of the alicyclic heterocyclic structure include:
oxygen atom-containing alicyclic heterocyclic structures such as oxirane, tetrahydrofuran, tetrahydropyran, dioxolane, and dioxane;
nitrogen atom-containing alicyclic heterocyclic structures such as aziridine, pyrrolidine, piperidine, and piperazine;
sulfur atom-containing alicyclic heterocyclic structures such as thietane, thiolane, and thiane; and
alicyclic heterocyclic structures containing a plurality of hetero atoms such as morpholine, 1,2-oxathiolane, and 1,3-oxathiolane.

The heterocyclic structures include a lactone structure, a cyclic carbonate structure, a sultone structure, a cyclic acetal, or a combination thereof.

When A includes a plurality of ring structures, the plurality of ring structures may form a spiro ring structure.

Among them, it is preferable that the first cyclic structure has an alicyclic hydrocarbon structure, a cyclic acetal structure, an aromatic ring structure, or a structure obtained by combining these structures from the viewpoint of appropriately controlling the acid diffusion length.

Preferable examples of the chain hydrocarbon group having 1 to 40 carbon atoms include groups corresponding to monovalent chain hydrocarbon groups having 1 to 20 carbon atoms represented by R¹⁸ to R²⁰ of the formula (4).

Examples of the substituent that substitutes some or all of the hydrogen atoms of the organic group include halogen atoms such as a fluorine atom, a chlorine atom, a bromine atom, and an iodine atom; a hydroxy group; a carboxy group; a cyano group; a nitro group; an amino group; an aldehyde group; a thiol group; and an oxo group (═O).

As the divalent heteroatom-containing group in the group containing the divalent heteroatom-containing group in a carbon-carbon bond of the group having a chain structure or the group having a cyclic structure, —CO—, —C(═O)O—, —CS—, —O—, —S—, —SO₂—, —NR″—, or a combination of two or more thereof can be suitably used. R″ represents a hydrogen atom or a monovalent hydrocarbon group having 1 to 5 carbon atoms. When the chain hydrocarbon group has the divalent heteroatom-containing groups, the number of the divalent heteroatom-containing groups is preferably 1, 2 or 3, and more preferably 1 or 2.

The (1+n)-valent organic group having 1 to 40 carbon atoms includes the chain structure, the first cyclic structure, or a combination thereof, and can be used without limitation as long as it is (1+n)-valent.

As the monovalent organic group represented by R^{f1} and R^{f2}, a monovalent organic group corresponding to the (1+n)-valent organic group having 1 to 40 carbon atoms represented by A can be suitably employed. Among them, a monovalent chain hydrocarbon group having 1 to 8 carbon atoms is preferable, and a monovalent linear hydrocarbon group having 1 to 5 carbon atoms is more preferable.

Examples of the monovalent fluorinated hydrocarbon groups represented by R^{f1} and R^{f2} include a monovalent fluorinated chain hydrocarbon group having 1 to 20 carbon atoms and a monovalent fluorinated alicyclic hydrocarbon group having 3 to 20 carbon atoms.

Examples of the monovalent fluorinated chain hydrocarbon group having 1 to 20 carbon atoms include:
fluorinated alkyl groups such as a trifluoromethyl group, a difluoromethyl group, a 2,2,2-trifluoroethyl group, a pentafluoroethyl group, a 2,2,3,3,3-pentafluoropropyl group, a 1,1,1,3,3,3-hexafluoropropyl group, a heptafluoro-n-propyl group, a heptafluoro-i-propyl group, a nonafluoro-n-butyl group, a nonafluoro-i-butyl group, a nonafluoro-t-butyl group, a 2,2,3,3,4,4,5,5-octafluoro-n-pentyl group, a tridecafluoro-n-hexyl group, and a 5,5,5-trifluoro-1,1-diethylpentyl group;
fluorinated alkenyl groups such as a trifluoroethenyl group and a pentafluoropropenyl group; and
fluorinated alkynyl groups such as a fluoroethynyl group and a trifluoropropynyl group.

Examples of the monovalent fluorinated alicyclic hydrocarbon group having 3 to 20 carbon atoms include:
fluorinated cycloalkyl groups such as a fluorocyclopentyl group, a difluorocyclopentyl group, a nonafluorocyclopentyl group, a fluorocyclohexyl group, a difluorocyclohexyl group, an undecafluorocyclohexylmethyl group, a fluoronorbornyl group, a fluoroadamantyl group, a fluorobornyl group, a fluoroisobornyl group, and a fluorotricyclodecyl group; and
fluorinated cycloalkenyl groups such as a fluorocyclopentenyl group and a nonafluorocyclohexenyl group.

The fluorinated hydrocarbon group is preferably a monovalent fluorinated chain hydrocarbon group having 1 to 8 carbon atoms, and more preferably a monovalent fluorinated linear hydrocarbon group having 1 to 5 carbon atoms.

In order for the first onium salt compound to sufficiently function as a radiation-sensitive acid generator, at least one of R^{f1} and R^{f2} is preferably a fluorine atom or a monovalent fluorinated hydrocarbon group, at least one of R^{f1} and R^{f2} on the carbon atom adjacent to the sulfur atom of the sulfonate ion (SO₃⁻) (at the α position with respect to the sulfur atom) is more preferably a fluorine atom or a monovalent fluorinated hydrocarbon group, and both of R^{f1} and R^{f2} on the carbon atom at the x position with respect to the sulfur atom of SO₃⁻ are still more preferably a fluorine atom or a monovalent fluorinated hydrocarbon group.

m1 is an integer of 1 to 8, and is preferably an integer of 2 to 6.

n is an integer of 1 to 3, preferably 1 or 2, and more preferably 1.

Specific examples of the first onium salt compound include, but are not limited to, structures represented by the formulas.

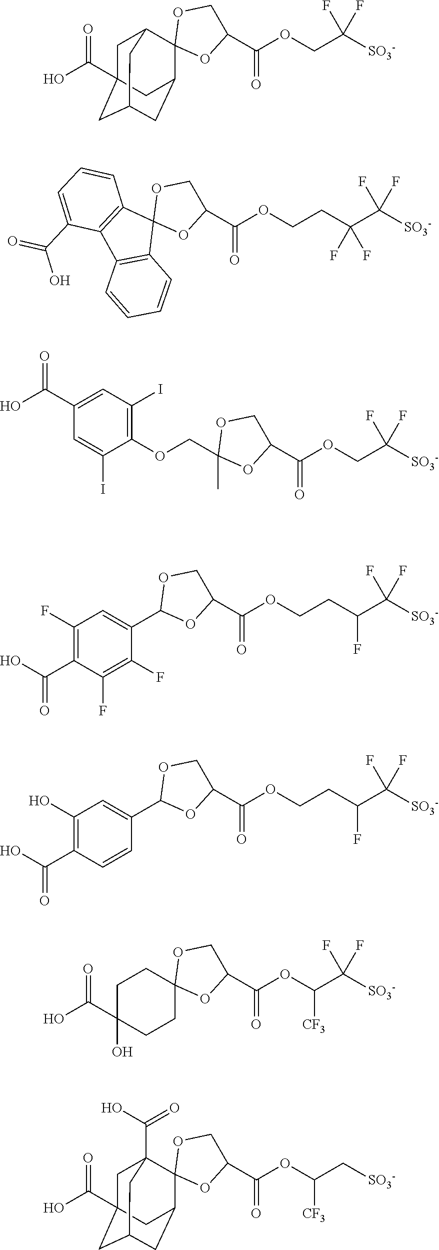

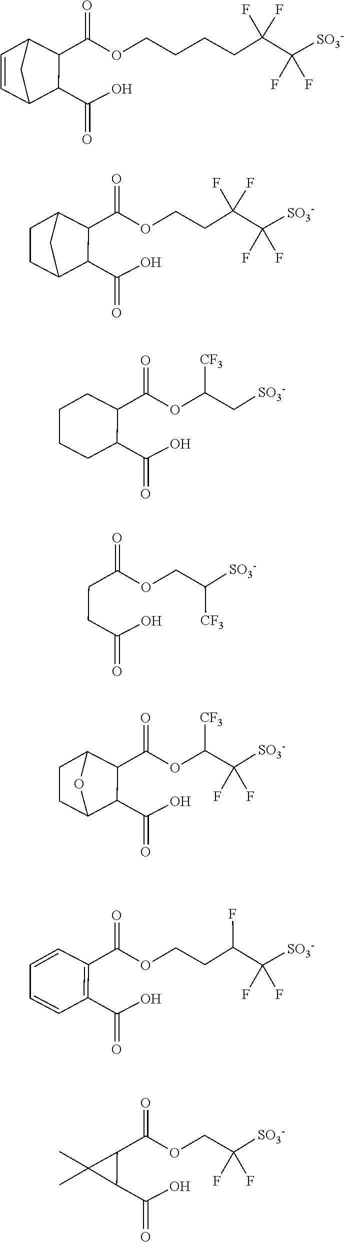

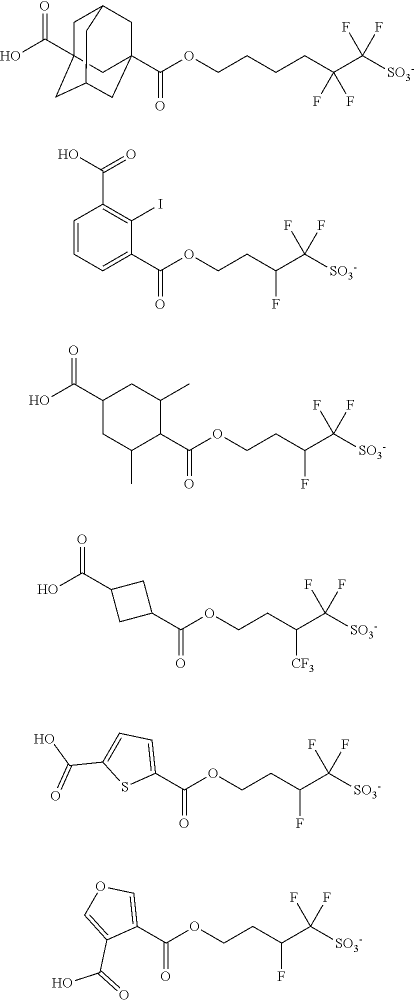

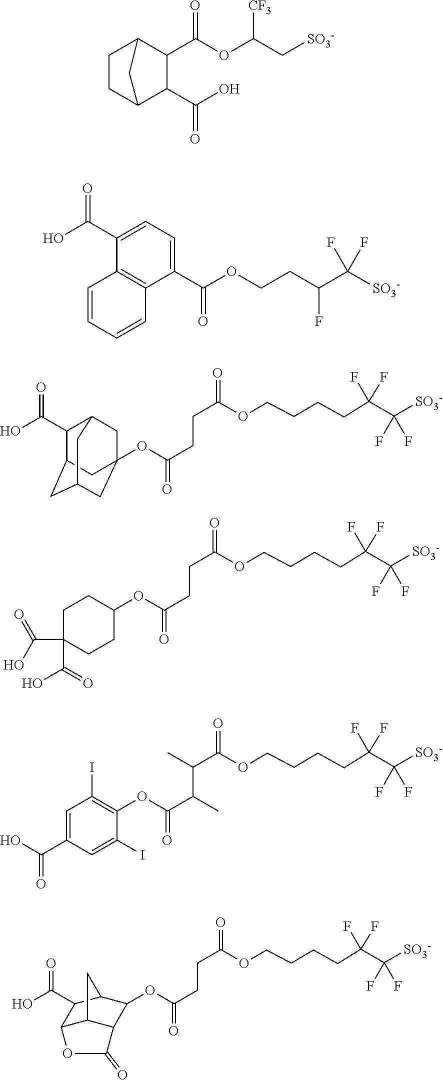

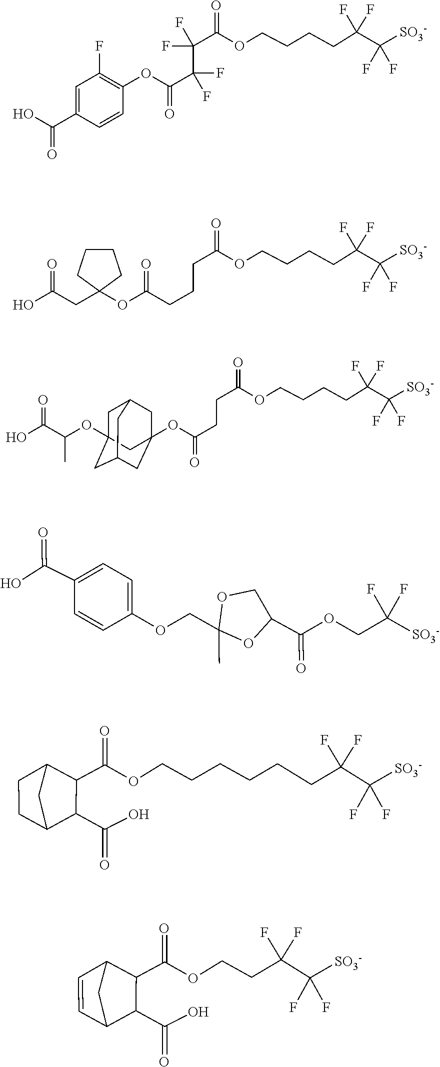

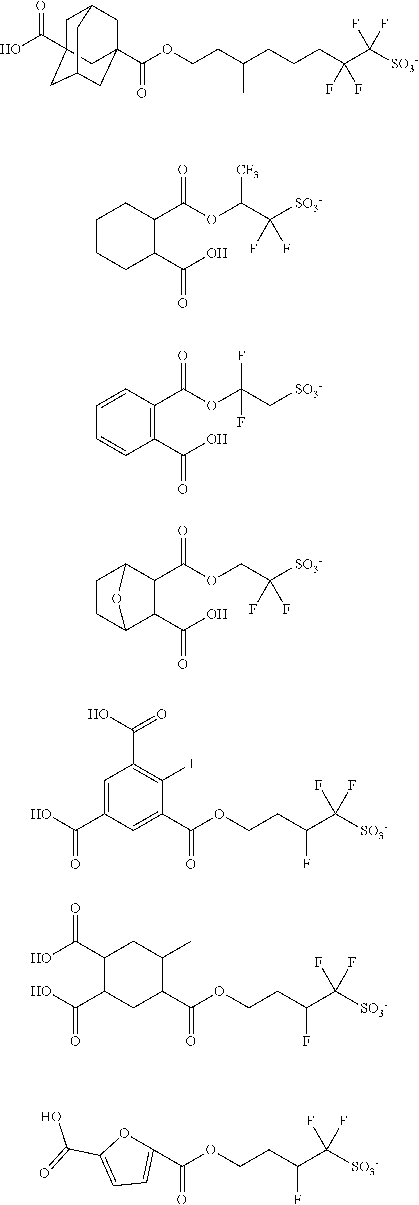

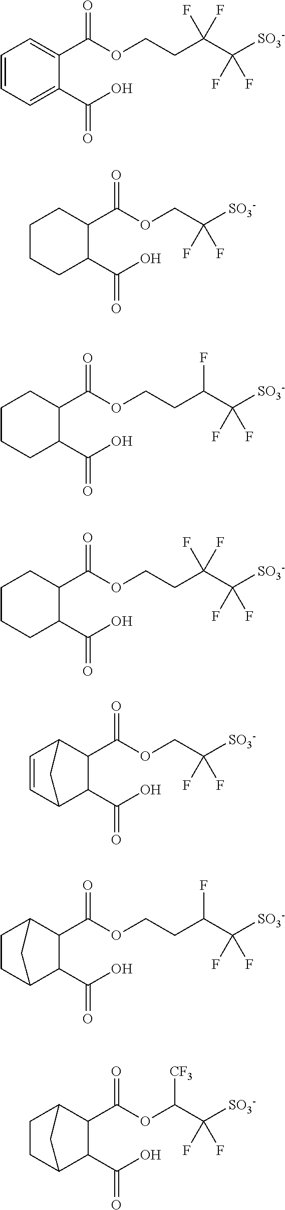

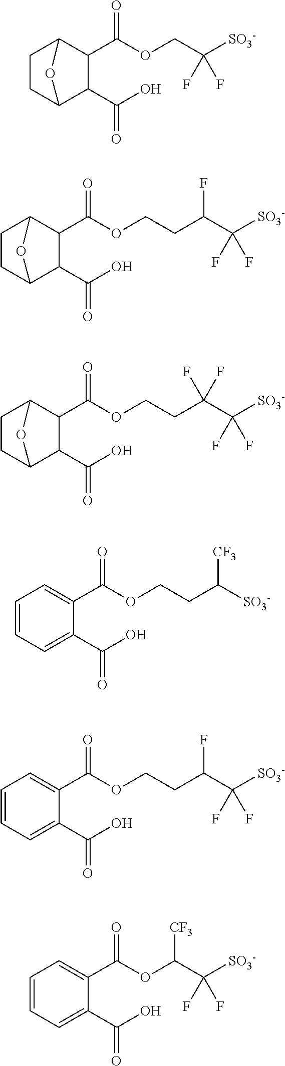

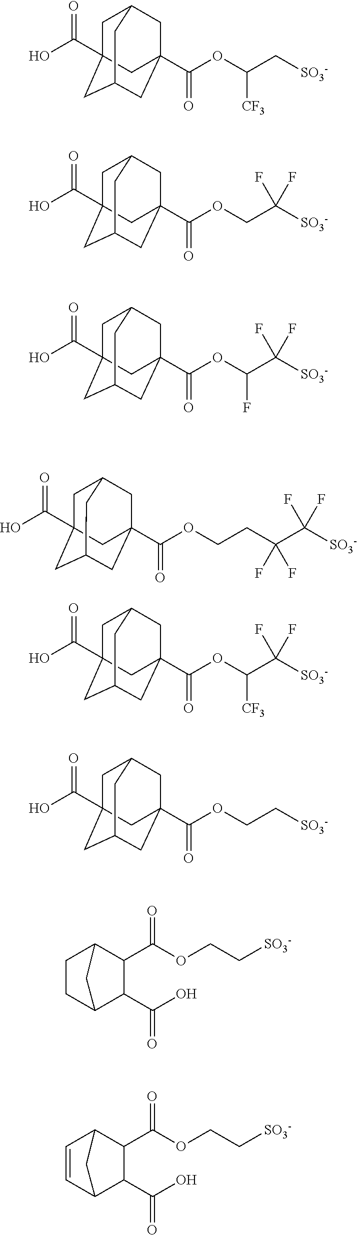

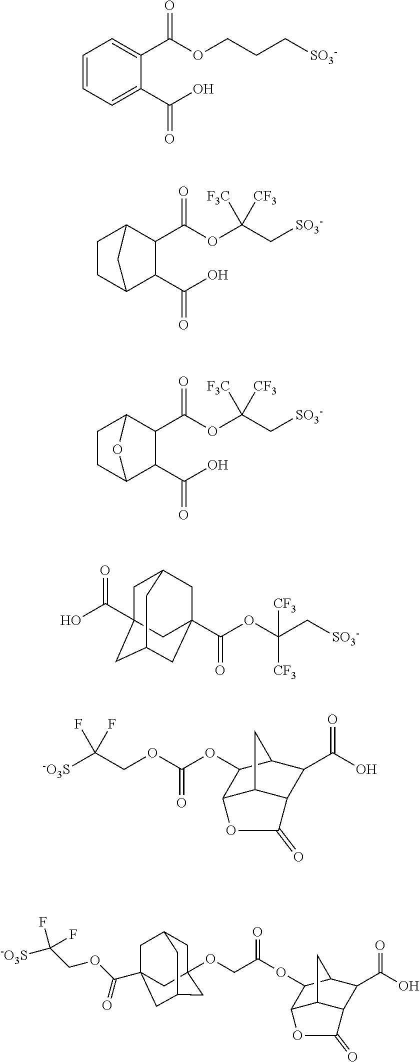

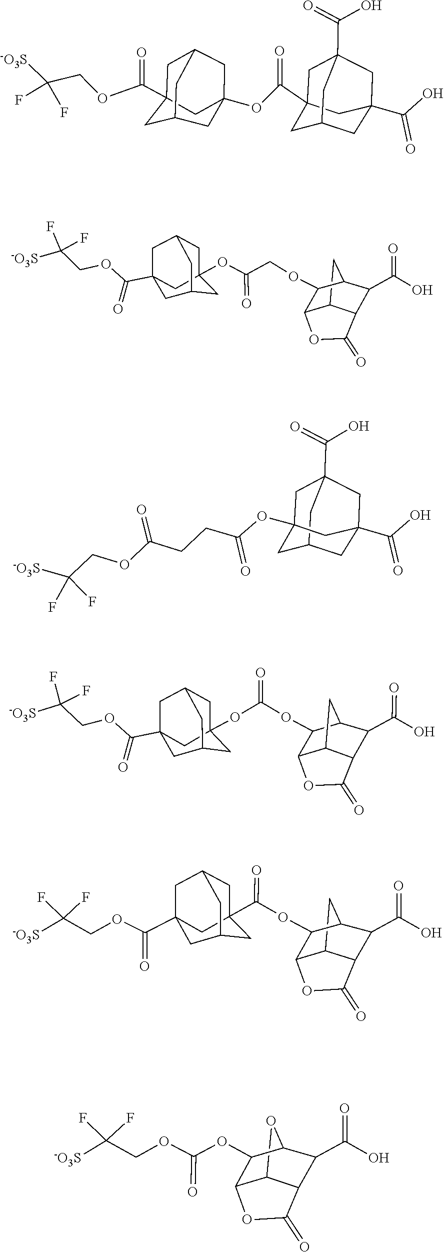

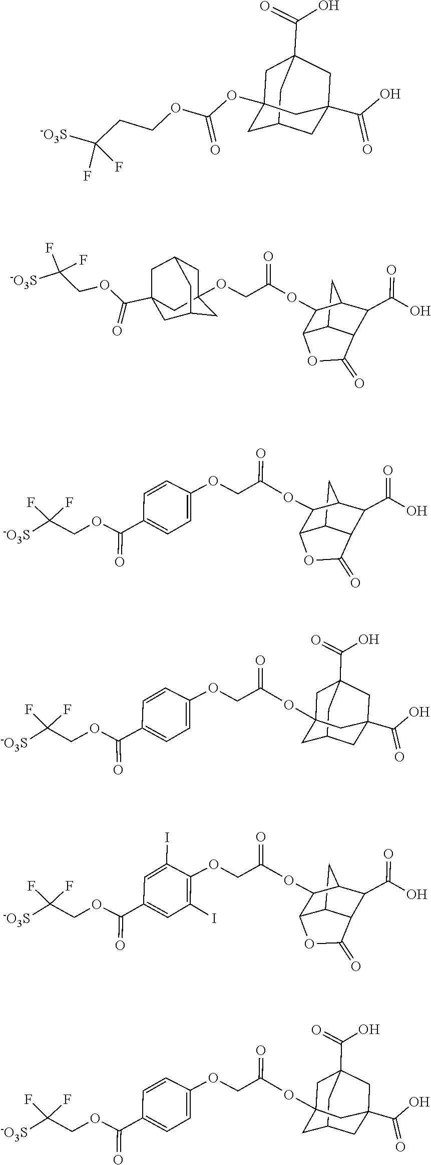

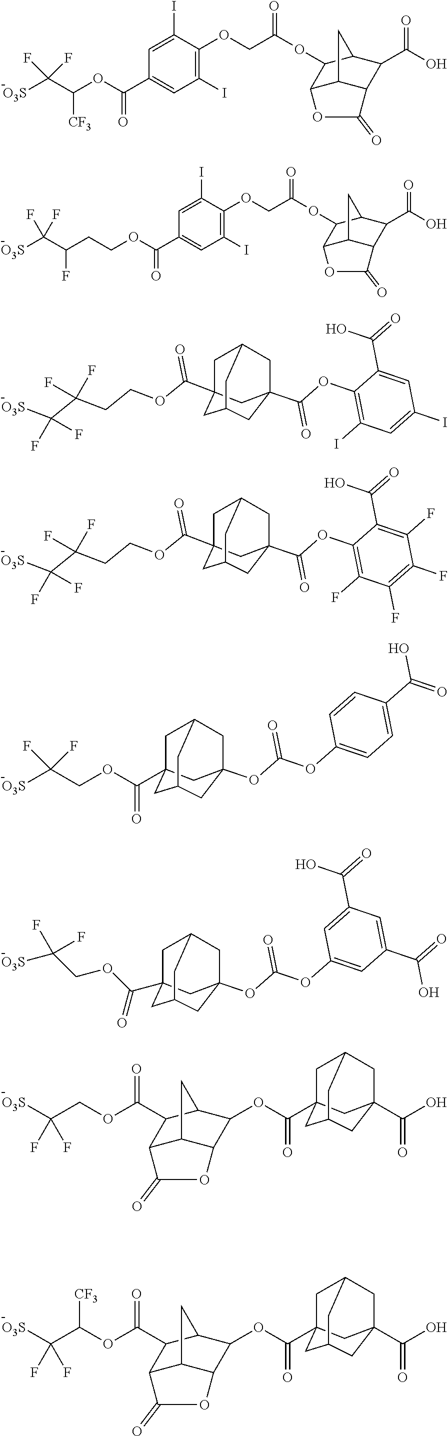

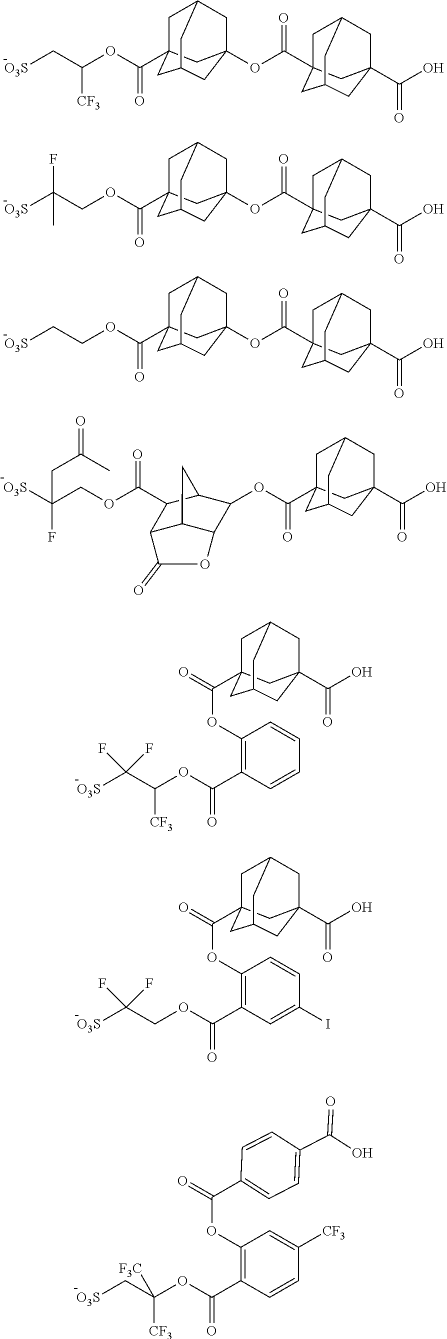

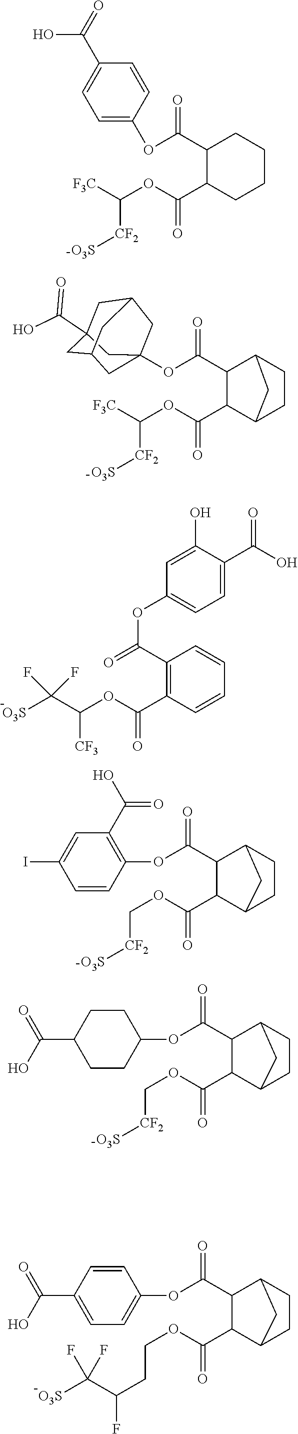

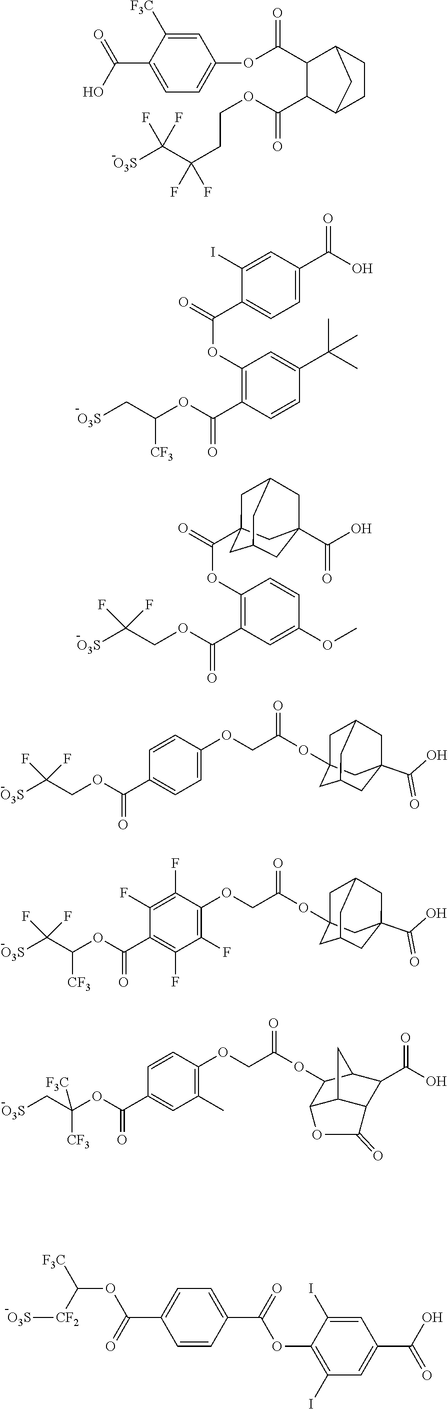

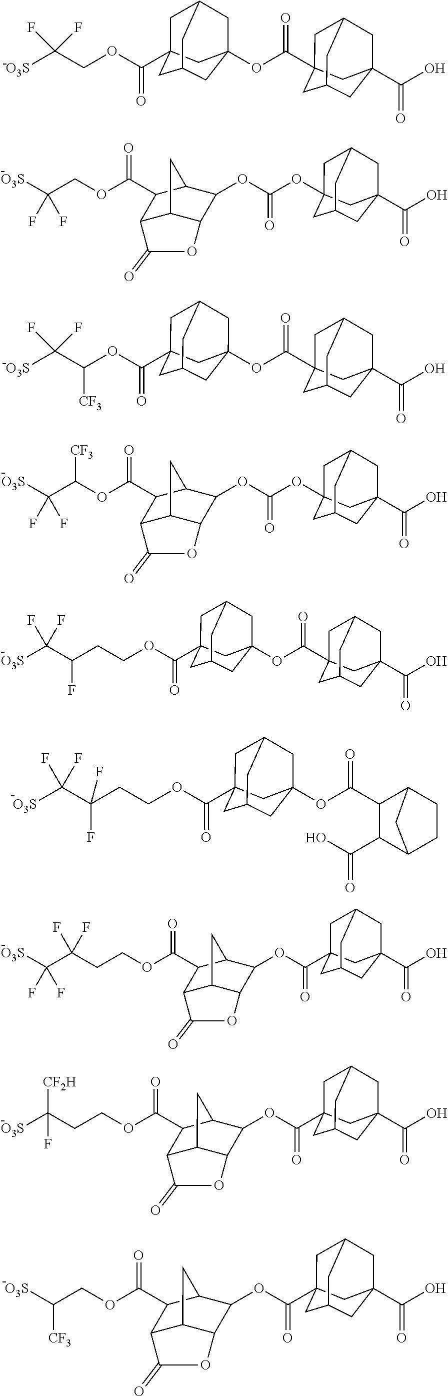

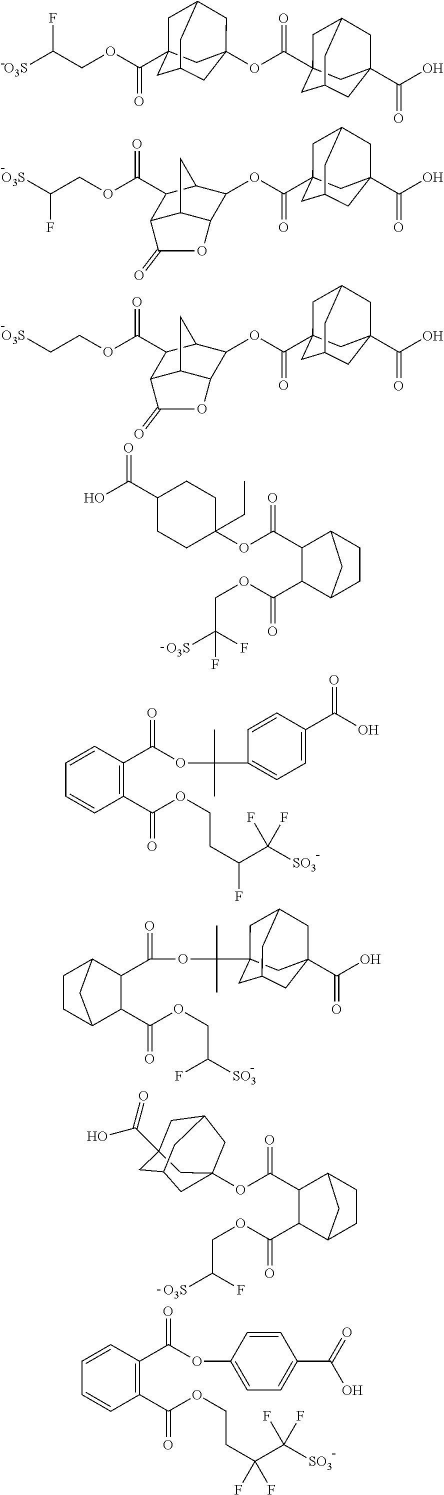

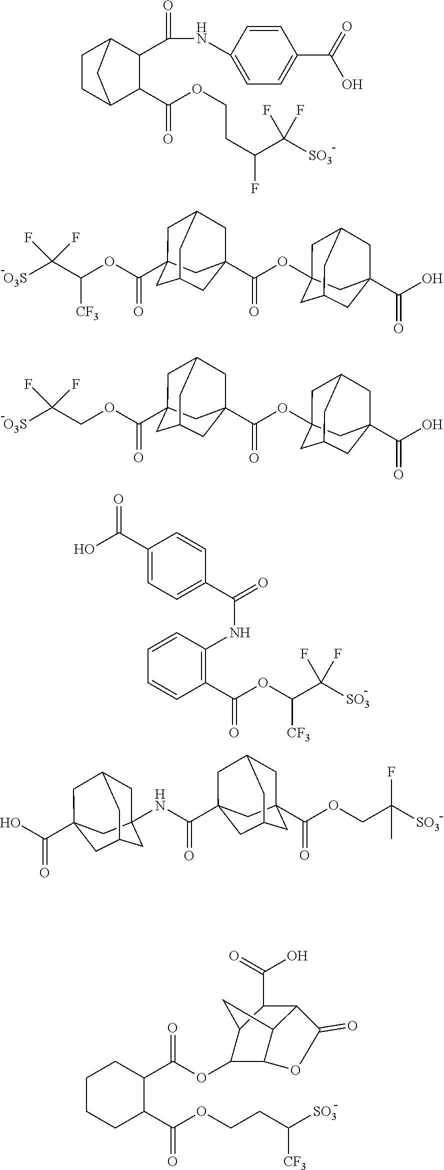

An example of the radiation-sensitive onium cation represented by Z₁⁺ of the formula (1) is a radioactive ray-degradable onium cation containing an element such as S, I, O, N, P, Cl, Br, F, As, Se, Sn, Sb, Te, or Bi. Examples of such a radioactive ray-degradable onium cation include a sulfonium cation, a tetrahydrothiophenium cation, a iodonium cation, a phosphonium cation, a diazonium cation, and a pyridinium cation. Among them, a sulfonium cation or a iodonium cation is preferred. The sulfonium cation or the iodonium cation is preferably represented by any of the formulas (X-1) to (X-6).

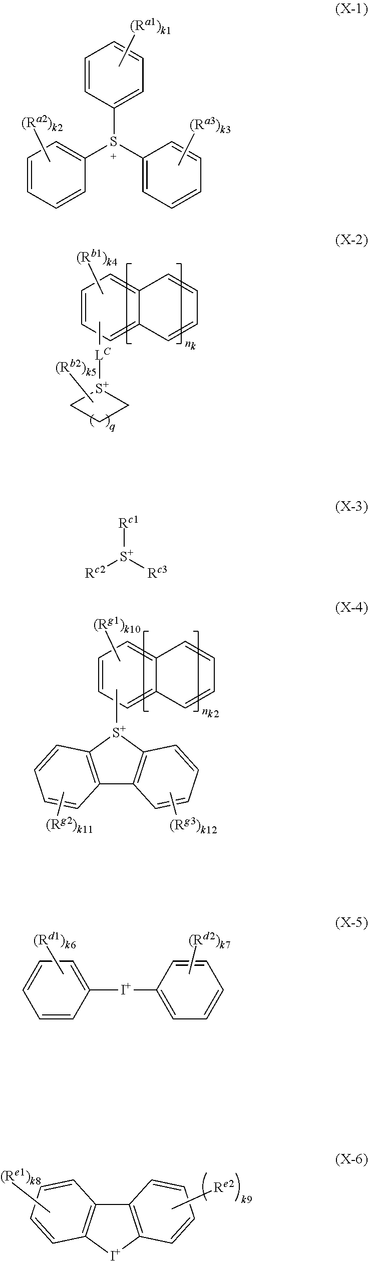

In the formula (X-1), R^{a1}, R^{a2} and R^{a3} are each independently a substituted or unsubstituted, linear or branched chain alkyl group, alkoxy group, or alkoxycarbonyloxy group having 1 to 12 carbon atoms; a substituted or unsubstituted, monocyclic or polycyclic cycloalkyl group having 3 to 12 carbon atoms; a substituted or unsubstituted aromatic hydrocarbon group having 6 to 12 carbon atoms; a hydroxy group, a halogen atom, —OSO₂—R^{P}, —SO₂—R^{Q}, —S—R^{T}, —O—, —CO— or a combination thereof; or a ring structure obtained by combining two or more of these groups. The ring structure may contain heteroatoms such as O and S between the carbon-carbon bonds forming the skeleton. R^{P}, R^{Q} and R^{T} are each independently a substituted or unsubstituted, linear or branched chain alkyl group having 1 to 12 carbon atoms; a substituted or unsubstituted alicyclic hydrocarbon group having 5 to 25 carbon atoms; or a substituted or unsubstituted aromatic hydrocarbon group having 6 to 12 carbon atoms. k1, k2 and k3 are each independently an integer of 0 to 5. When there are a plurality of R^{a1} to R^{a3} and a plurality of R^{P}, R^{Q} and R^{T}, a plurality of R^{a1} to R^{a3} and a plurality of R^{P}, R^{Q} and R^{T} may be each identical or different.

In the formula (X-2), R^{b1} is a substituted or unsubstituted, linear or branched chain alkyl group or alkoxy group having 1 to 20 carbon atoms; an alkoxyalkyl group; a substituted or unsubstituted acyl group having 2 to 8 carbon atoms; or a substituted or unsubstituted aromatic hydrocarbon group having 6 to 8 carbon atoms; or a hydroxy group. nₖ is 0 or 1. When nₖ is 0, k4 is an integer of 0 to 4. When nₖ is 1, k4 is an integer of 0 to 7. When there are a plurality of R^{b1}, a plurality of R^{b1} may be each identical or different. A plurality of R^{b1} may represent a ring structure obtained by combining them. R^{b2} is a substituted or unsubstituted, linear or branched chain alkyl group having 1 to 7 carbon atoms; or a substituted or unsubstituted aromatic hydrocarbon group having 6 or 7 carbon atoms. LC is a single bond or divalent linking group. k5 is an integer of 0 to 4. When there are a plurality of R^{b2}, a plurality of R^{b2} may be each identical or different. A plurality of R^{b2} may represent a ring structure obtained by combining them. q is an integer of 0 to 3. In the formula, the ring structure containing S⁺ may contain a heteroatom such as O or S between the carbon-carbon bonds forming the skeleton.

In the formula (X-3), R^{c1}, R^{c2} and R^{c3} are each independently a substituted or unsubstituted, linear or branched chain alkyl group having 1 to 12 carbon atoms.

In the formula (X-4), R^{g1} is a substituted or unsubstituted linear or branched chain alkyl or alkoxy group having 1 to 20 carbon atoms, a substituted or unsubstituted acyl group having 2 to 8 carbon atoms, a substituted or unsubstituted aromatic hydrocarbon group having 6 to 8 carbon atoms, or a hydroxy group. nₖ₂ is 0 or 1. When nₖ₂ is 0, k10 is an integer of 0 to 4, and when nₖ₂ is 1, k10 is an integer of 0 to 7. When there are two or more R^{g1}s, the two or more

Rols are the same or different from each other, and may represent a cyclic structure formed by combining them together. R^{g2} and R^{g3} are each independently a substituted or unsubstituted linear or branched chain alkyl, alkoxy, or alkoxycarbonyloxy group having 1 to 12 carbon atoms, a substituted or unsubstituted monocyclic or polycyclic cycloalkyl group having 3 to 12 carbon atoms, a substituted or unsubstituted aromatic hydrocarbon group having 6 to 12 carbon atoms, a hydroxyl group, a halogen atom, or a ring structure formed by combining two or more of these groups together. K11 and k12 are each independently an integer of 0 to 4. When there are two or more R^{g2}s and two or more R^{g3}s, the two or more R^{g2}s may be the same or different from each other, and the two or more R^{g3}s may be the same or different from each other.

In the formula (X-5), R^{d1} and R^{d2} are each independently a substituted or unsubstituted, linear or branched chain alkyl group, alkoxy group or alkoxycarbonyl group having 1 to 12 carbon atoms; a substituted or unsubstituted aromatic hydrocarbon group having 6 to 12 carbon atoms; a halogen atom; a halogenated alkyl group having 1 to 4 carbon atoms; a nitro group; or a ring structure obtained by combining two or more of these groups. k6 and k7 are each independently an integer of 0 to 5. When there are a plurality of R^{d1} and a plurality of R^{d2}, a plurality of R^{d1} and a plurality of R^{d2} may be each identical or different.

In the formula (X-6), R^{e1} and R^{e2} are each independently a halogen atom; a substituted or unsubstituted linear or branched chain alkyl group having 1 to 12 carbon atoms; or a substituted or unsubstituted aromatic hydrocarbon group having 6 to 12 carbon atoms. k8 and k9 are each independently an integer of 0 to 4.

Specific examples of the radiation-sensitive onium cation include, but not limited thereto, the structures represented by the formulas (1-2-1) to (Jan. 2, 1959).

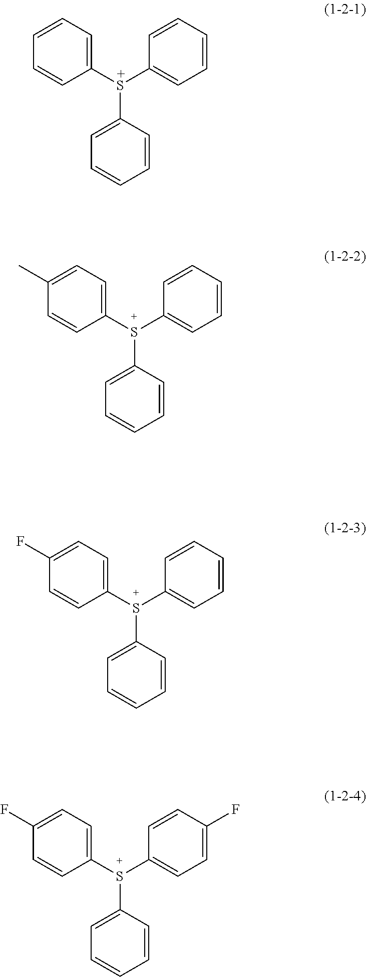

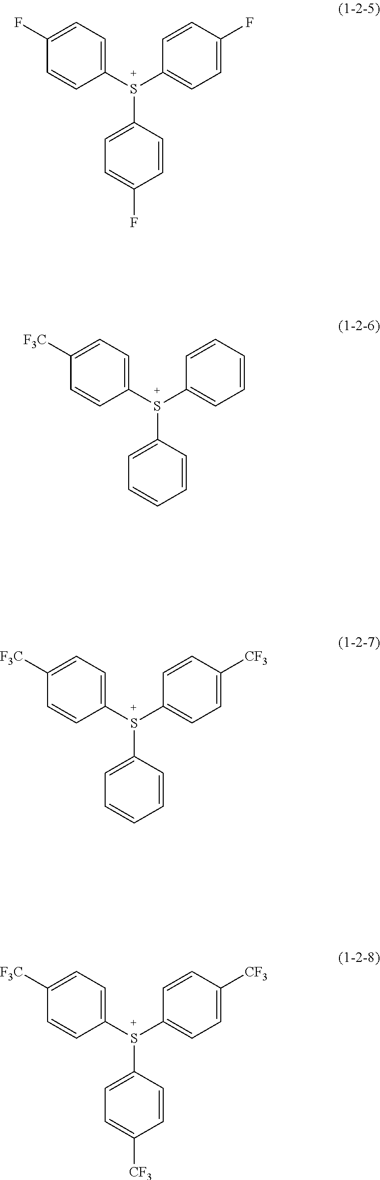

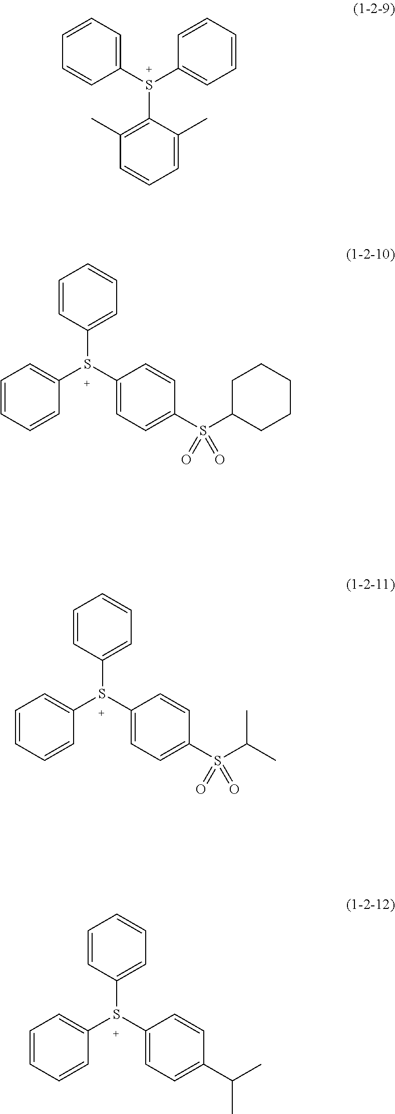

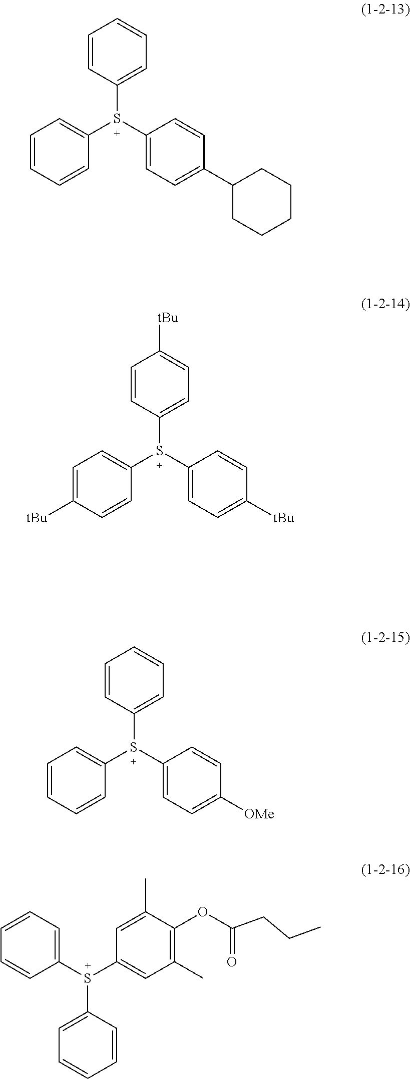

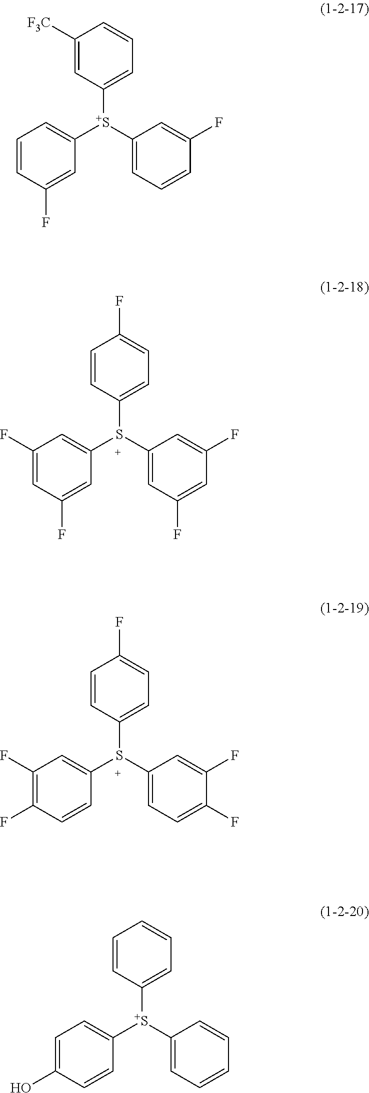

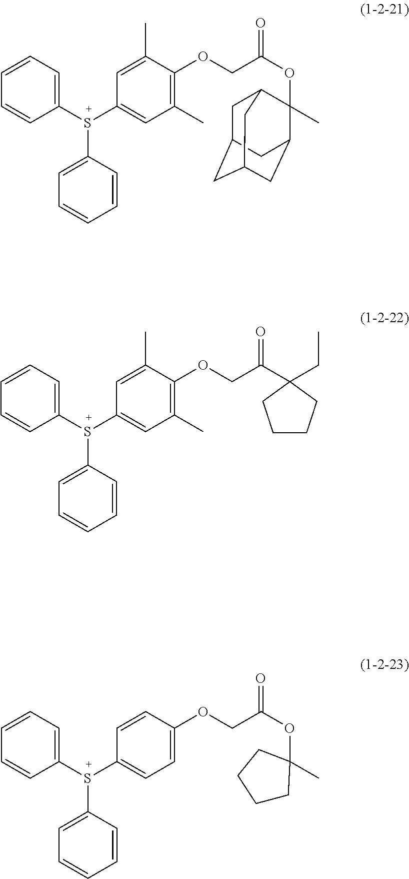

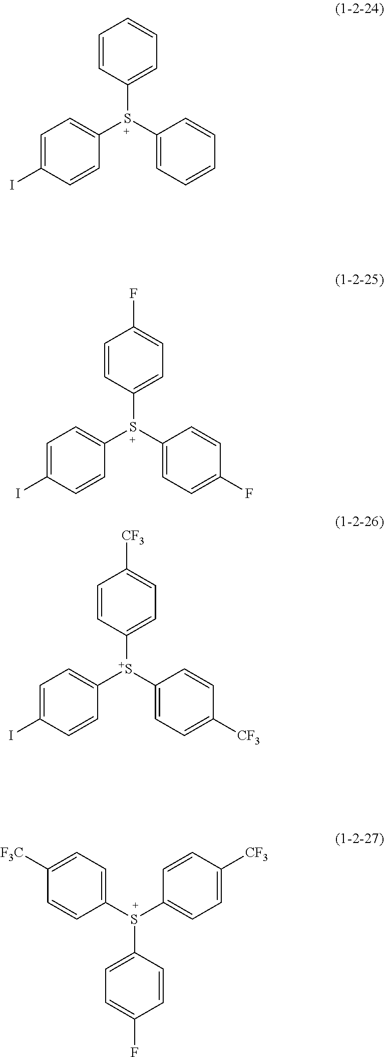

In the formula, tBu represents a t-butyl group, and Me represents a methyl group.

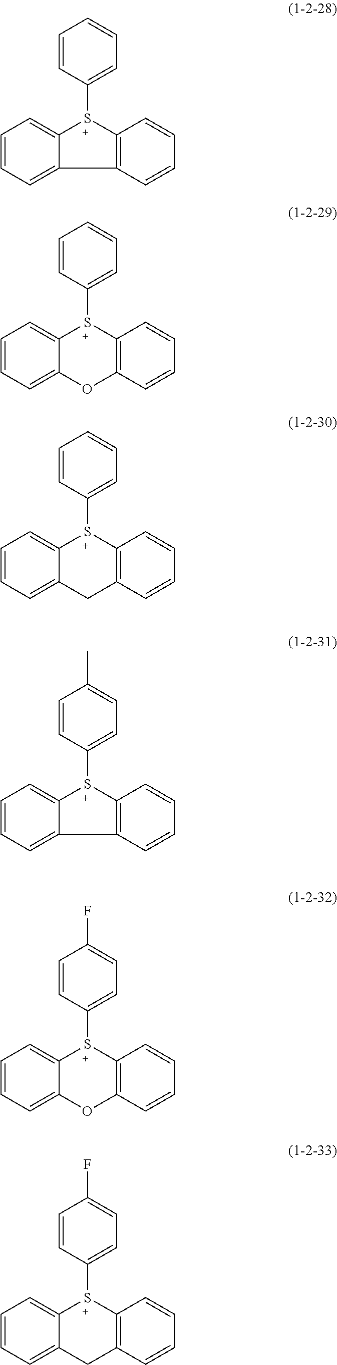

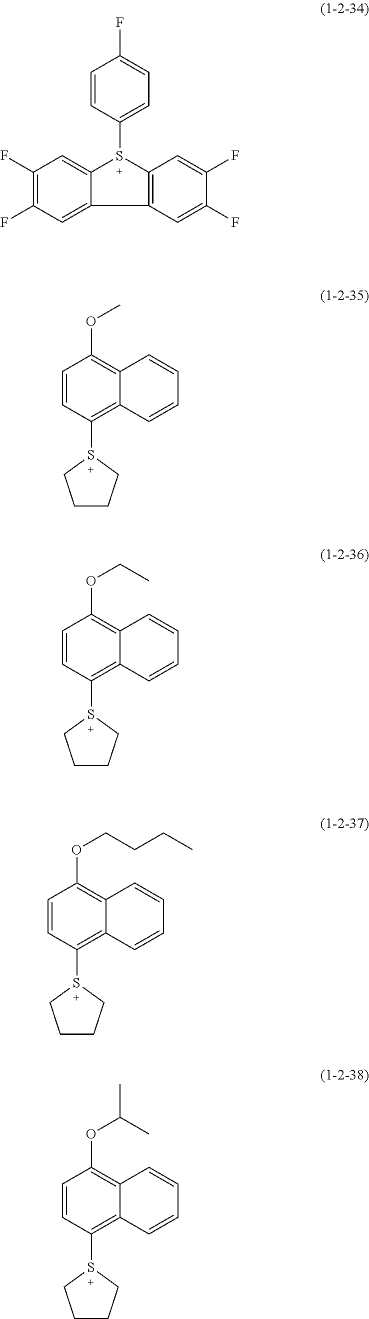

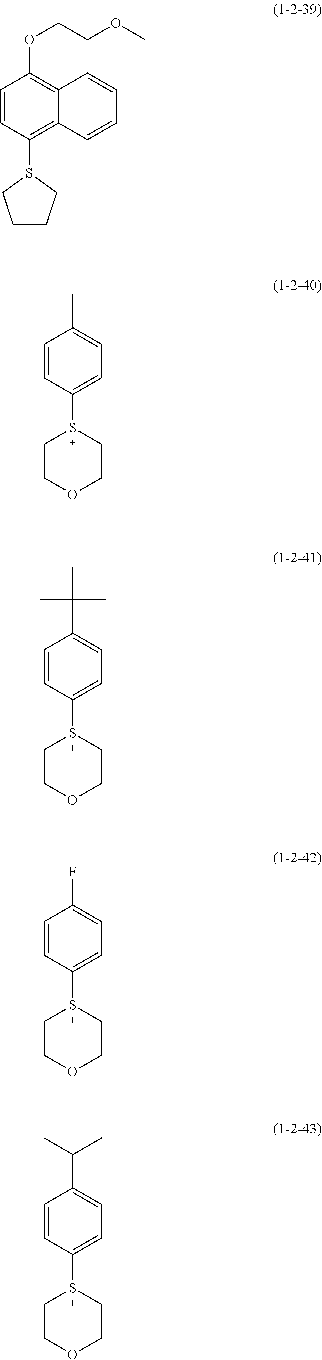

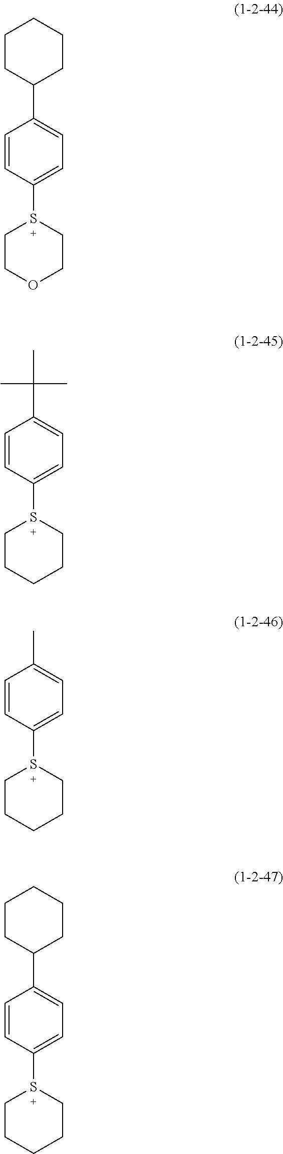

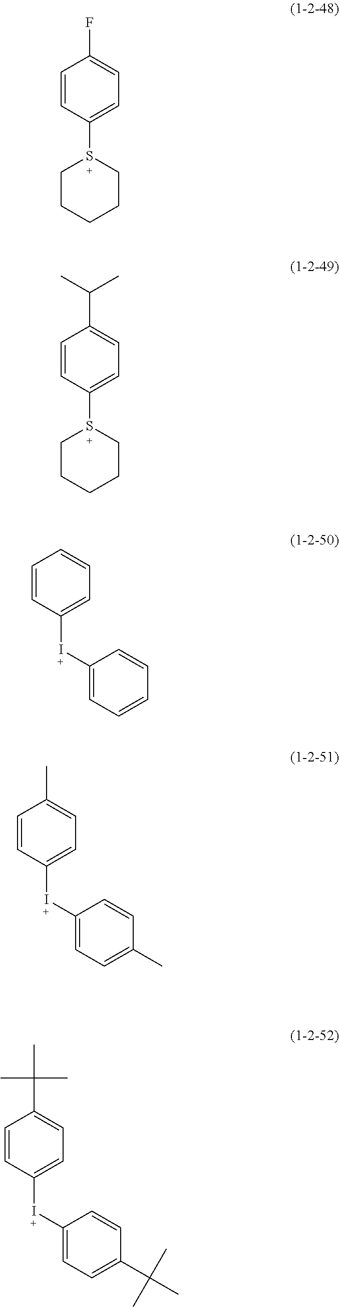

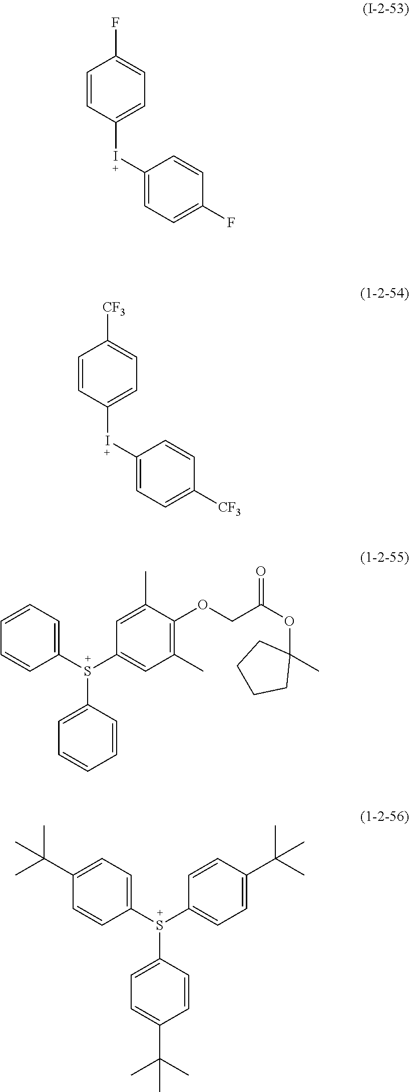

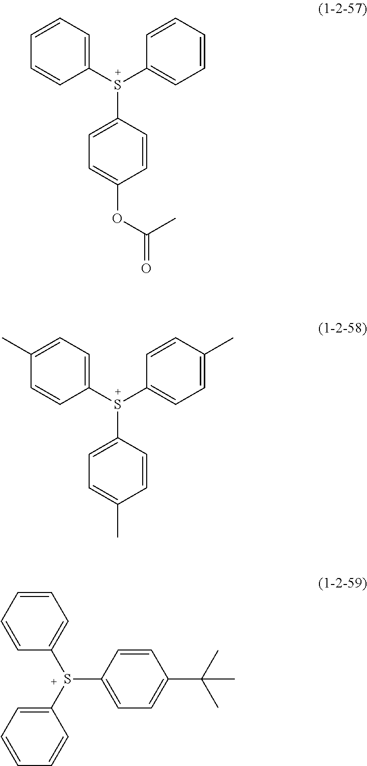

The first onium salt compound as a radiation-sensitive acid generator is obtained by appropriately combining the aforementioned anions and the aforementioned radiation-sensitive onium cations. Specific examples thereof include, but are not particularly limited to, structures represented by formulae (1-3-1) to (Jan. 3, 2028).

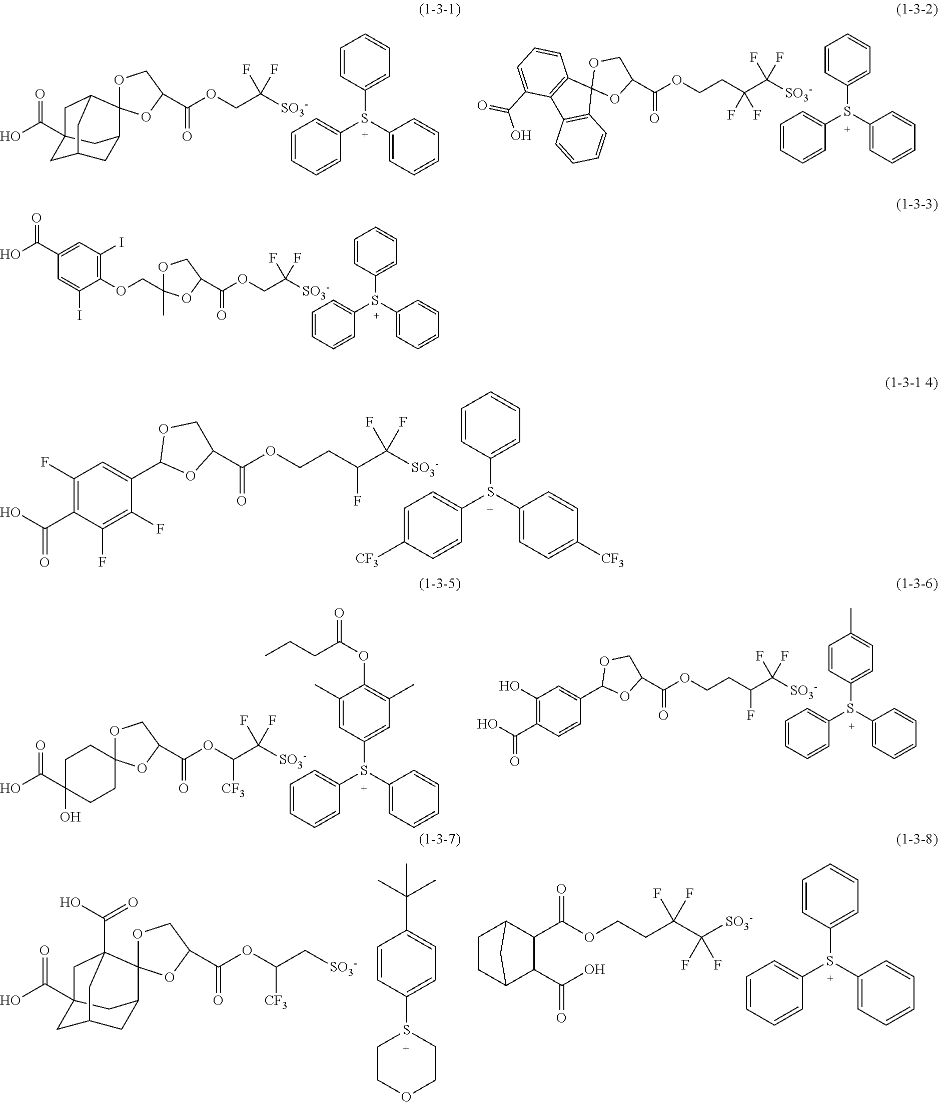

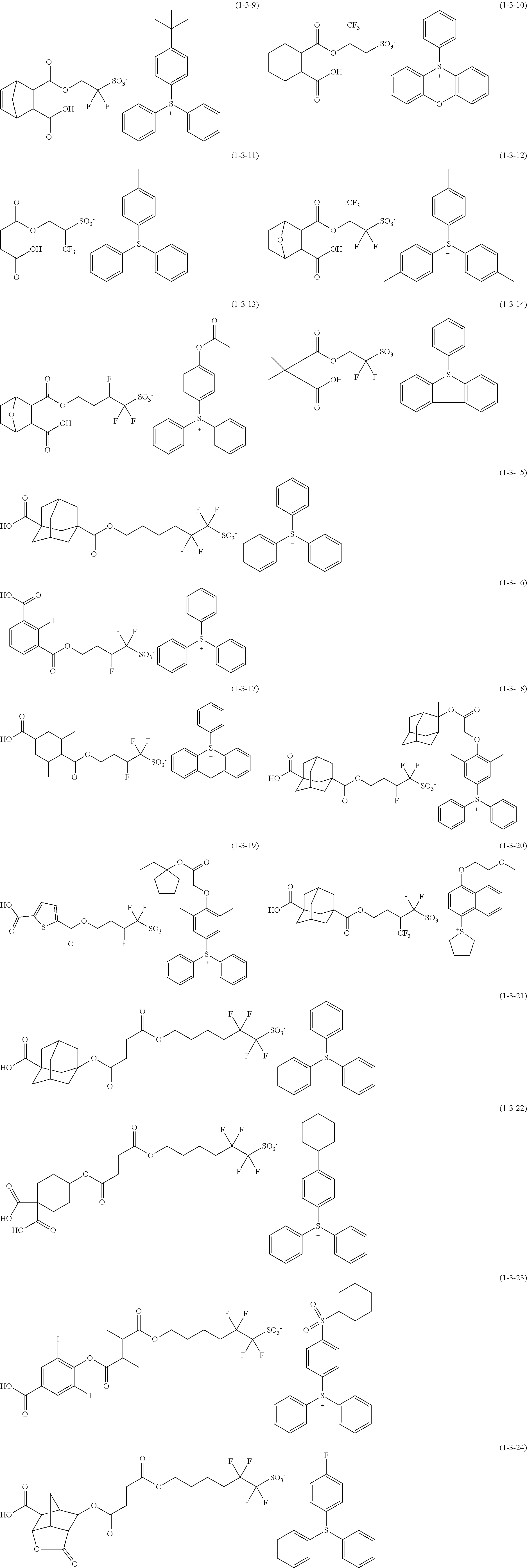

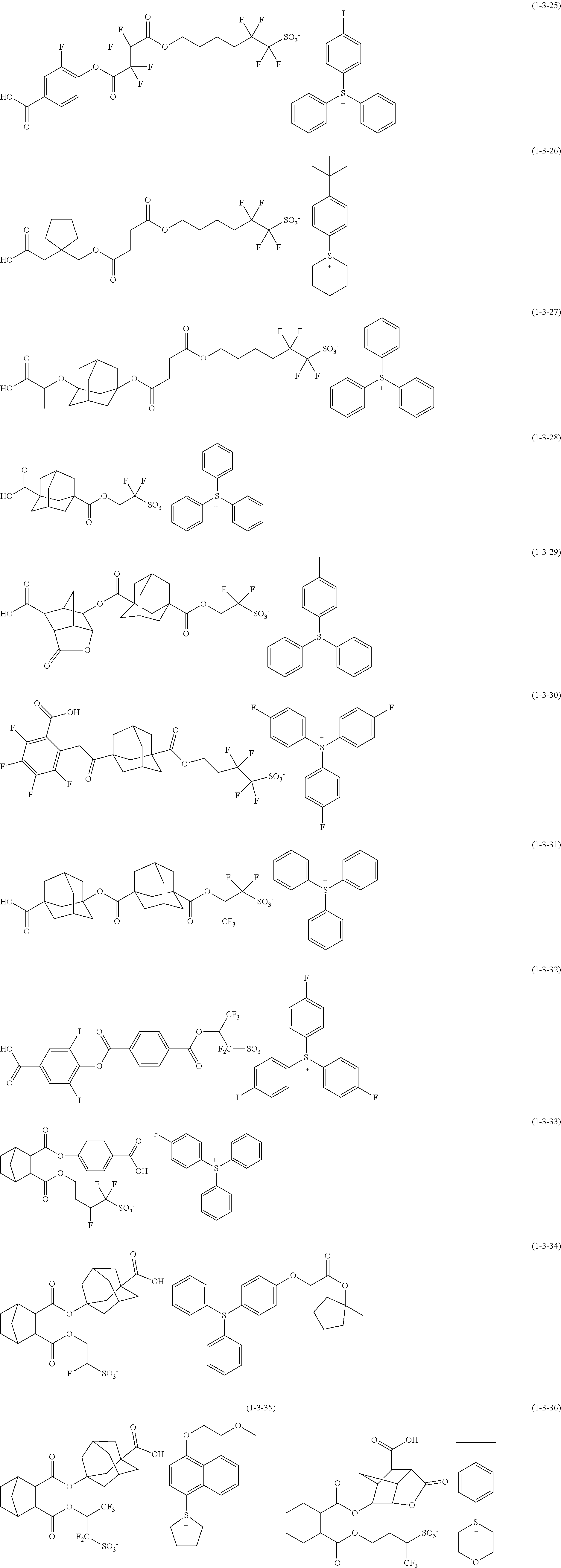

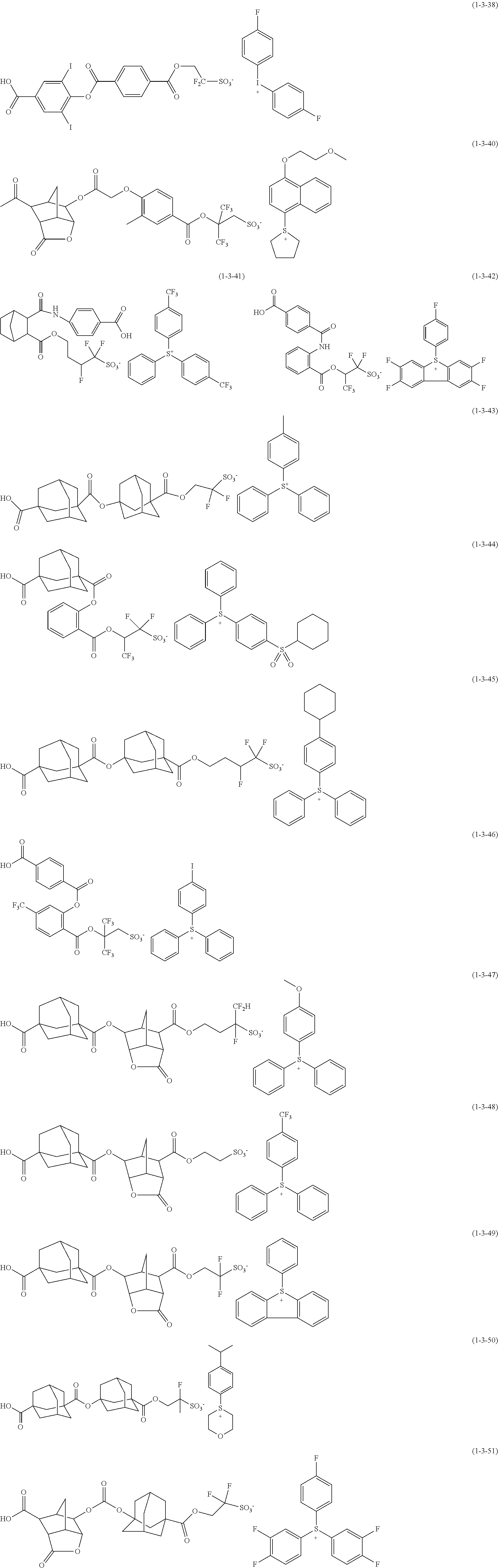

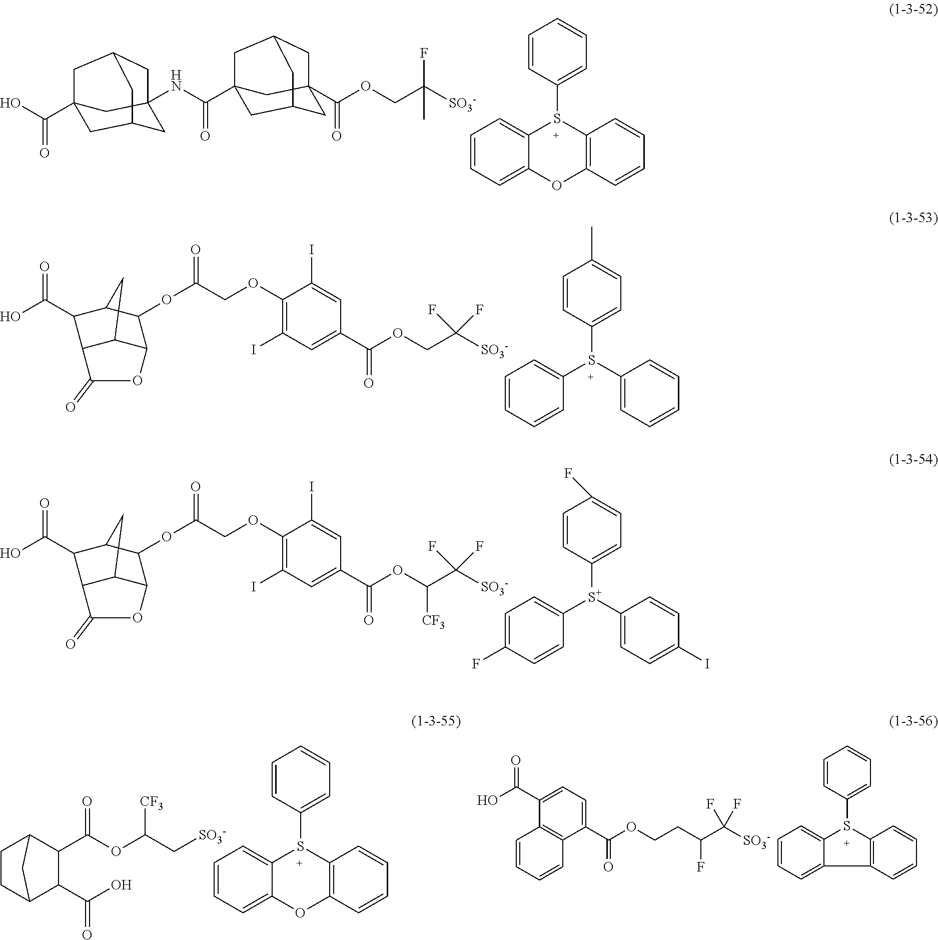

The lower limit of the content of the first onium salt compound (when plural kinds of first onium salt compounds are contained, the total content thereof) is preferably 1 part by mass, more preferably 3 parts by mass, and still more preferably 5 parts by mass based on 100 parts by mass of the polymer (A). The upper limit of the content is preferably 60 parts by mass, more preferably 40 parts by mass, and still more preferably 30 parts by mass. The content of the first onium salt compound is appropriately selected according to the type of a polymer to be used, exposure conditions, required sensitivity, and the like. This makes it possible to exhibit superior sensitivity, LWR performance, and pattern rectangularity when forming a resist pattern.

The first onium salt compound as a radiation-sensitive acid generator and another radiation-sensitive acid generator (for example, the onium salt compound (P1) described below) may be used in combination.

(Radiation-Sensitive Acid Generator (P1) Other than First Onium Salt Compound)

The radiation-sensitive composition may include a radiation-sensitive acid generator other than the first onium salt compound as a radiation-sensitive acid generator.

Examples of the radiation-sensitive acid generator include an onium salt compound (P1) represented by the following formula (P1), provided that those corresponding to the first onium salt compound are excluded.

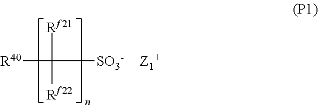

in the formula (P1),
R⁴⁰ is a monovalent organic group having 3 to 40 carbon atoms,
R^{f21} and R^{f22} each independently represent a fluorine atom or a monovalent fluorinated hydrocarbon group, when there are a plurality of R^{f21}s and R^{f22}s, the plurality of R^{f21}s and R^{f22}s are the same or different from each other;
n is an integer of 1 to 4; and
Z₂⁺ represents a radiation-sensitive onium cation.

As the monovalent organic group having 3 to 40 carbon atoms represented by R⁴⁰, a monovalent organic group corresponding to the (1+n)-valent organic group having 1 to 40 carbon atoms represented by A of the formula (1) can be suitably employed.

As the monovalent fluorinated hydrocarbon group represented by R^{f21} and R^{f22}, monovalent fluorinated hydrocarbon groups represented by R^{f1} and R^{f2} of the formula (1) can be suitably employed.

Specific examples of the anion of the onium salt compound (P1) include, but are not limited to, structures represented by the formulas.

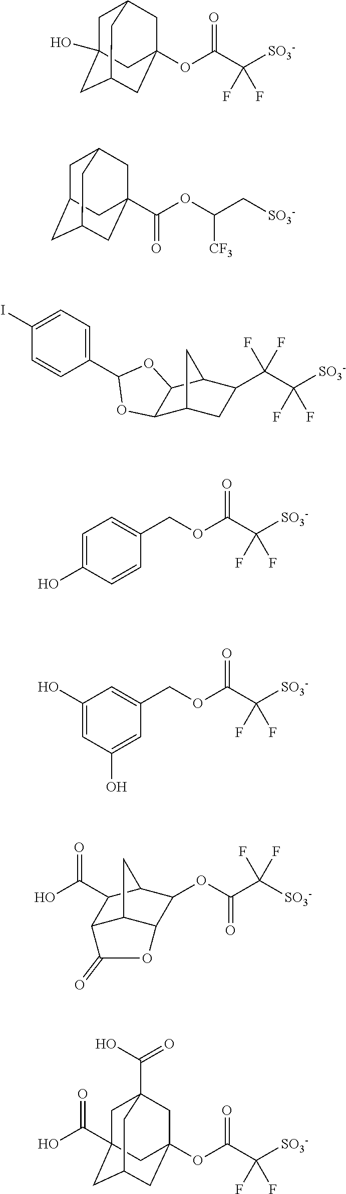

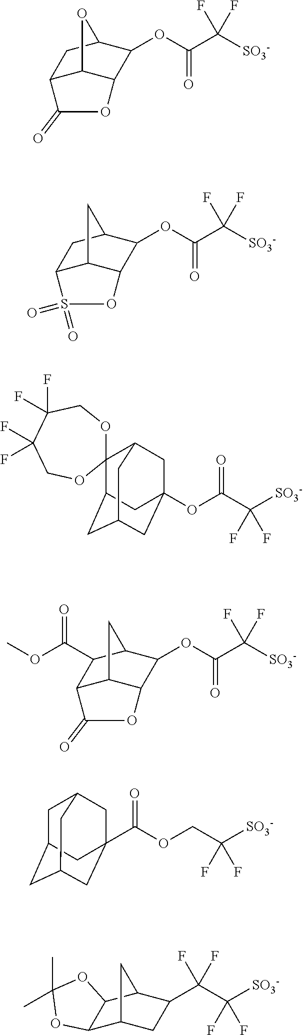

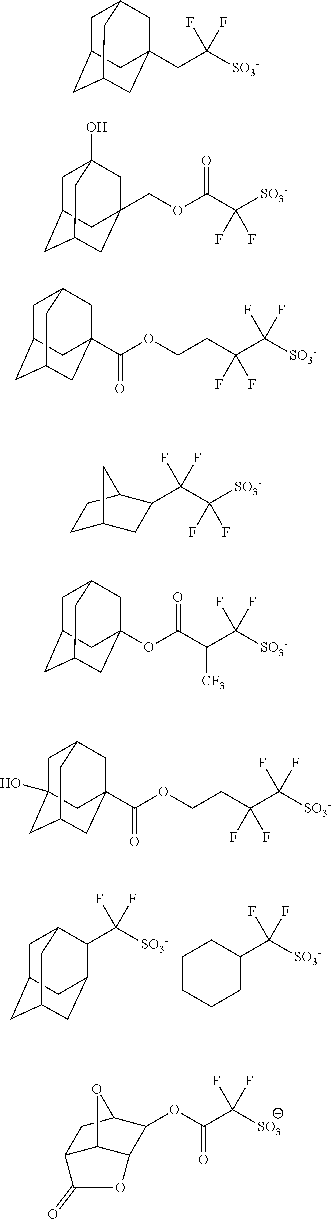

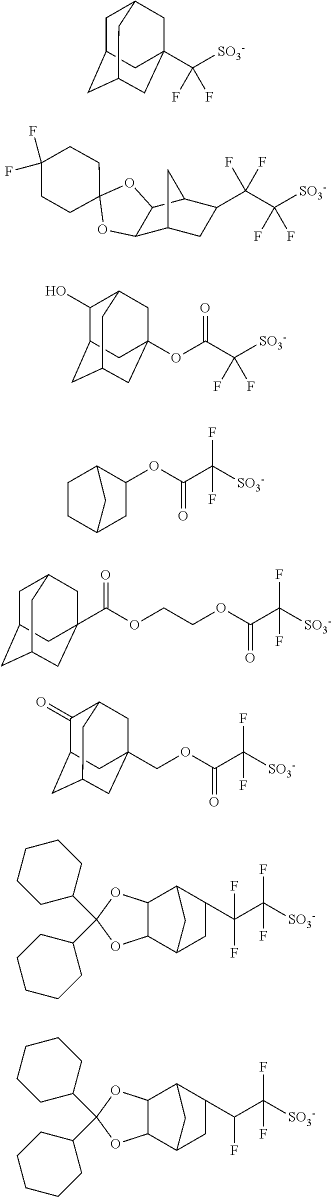

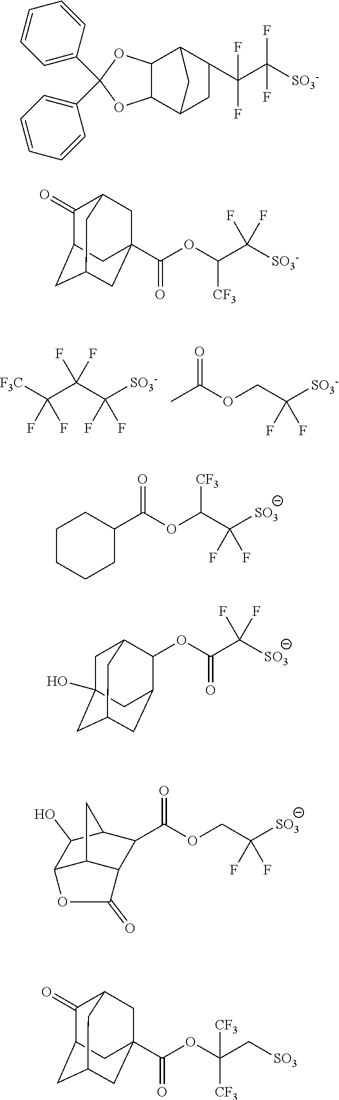

Specific examples of the radiation-sensitive onium cation of the onium salt compound (P1) are not limited, but the structures exemplified as the specific examples of the radiation-sensitive onium cation of the formula (1) can be suitably employed.

Examples of the onium salt compound (P1) include structures obtained by arbitrarily combining the anions and the radiation-sensitive onium cations.

As the radiation-sensitive acid generator other than the first onium salt compound, an intramolecular salt compound (P2) containing a cation and an anion in the same molecule represented by formula (Y-1) below can also be used.

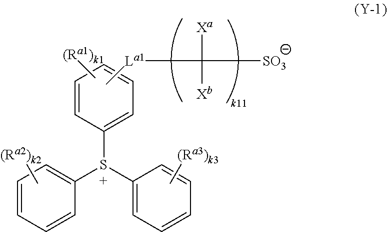

In the formula (Y-1), R^{a1}, R^{a2}, R^{a3}, k1, k2, and k3 have the same meaning as in the formula (X-1). L^{a1} is a single bond or a divalent linking group. X^{a} and X^{b} are each independently a hydrogen atom, a fluorine atom, or a trifluoromethyl group. k11 is an integer of 1 to 4.

As the divalent linking group, for example, a divalent linking group represented by L² of the formulas (T-1) to (T-10) and the divalent heteroatom-containing group can be suitably employed.

Specific examples of the intramolecular salt compound represented by formula (Y-1) include, but are not particularly limited to, structures represented by formulae (1-4-1) to (1-4-9).

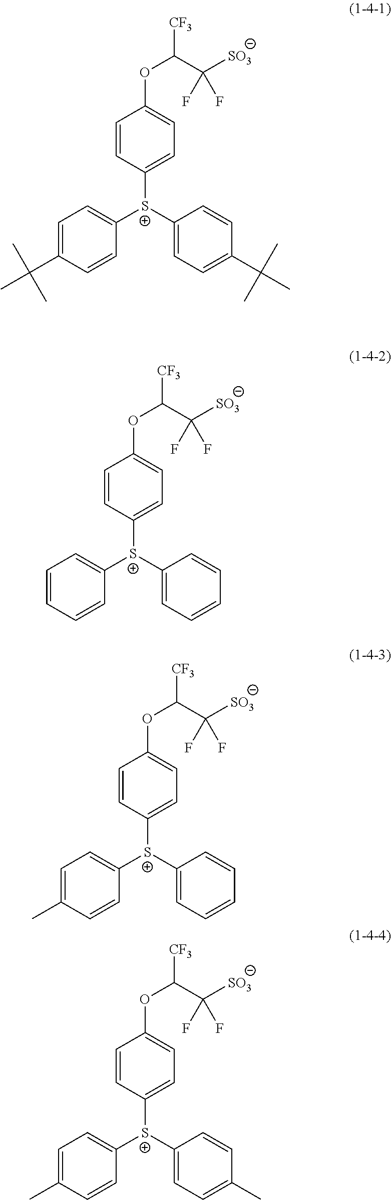

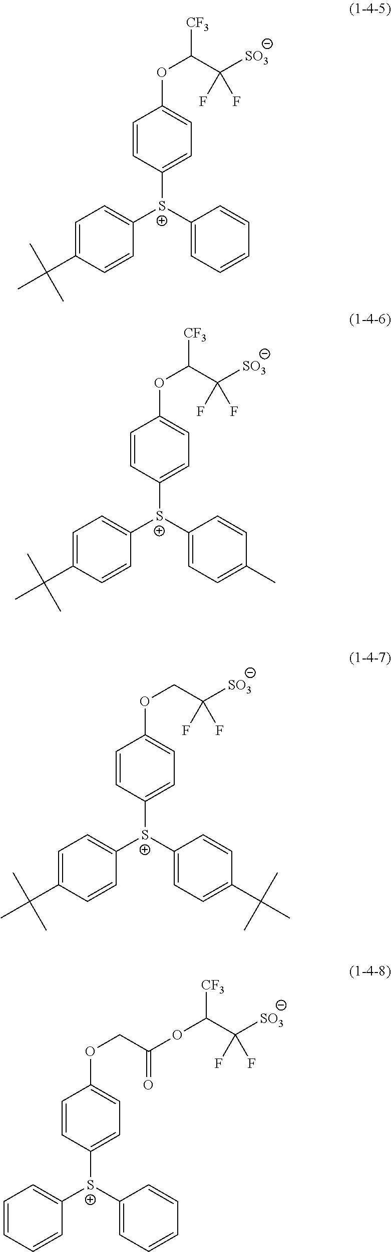

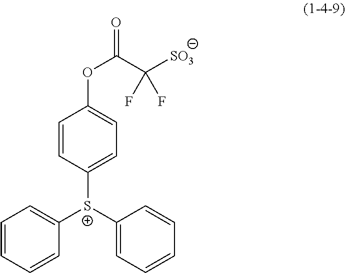

The lower limit of the content of the radiation-sensitive acid generator other than the first onium salt compound (when plural kinds of onium salt compounds (P1) and the intramolecular salt compounds (P2) are contained, the total content thereof) is preferably 0 parts by mass, more preferably 0.1 parts by mass, still more preferably 0.5 parts by mass, particularly preferably 3 parts by mass with respect to 100 parts by mass of the polymer (A). The upper limit of the content is preferably 50 parts by mass, more preferably 40 parts by mass, still more preferably 30 parts by mass, and particularly preferably 25 parts by mass. The content of the radiation-sensitive acid generator other than the first onium salt compound is appropriately selected depending on the type of a polymer to be used, exposure conditions, required sensitivity, and the like.

### (Second Onium Salt Compound)

The second onium salt compound is at least one selected from the group consisting of a carboxylic acid salt compound represented by formula (2) below and a carboxylic acid intramolecular salt compound represented by formula (3) below;

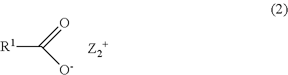

wherein, in the formula (2),
R¹ is a monovalent organic group having 2 to 40 carbon atoms; and
Z₂⁺ represents an organic cation, and

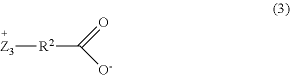

wherein, in the formula (3),
R² is a single bond or a divalent organic group having 1 to 40 carbon atoms; and
Z₃⁺ is a monovalent organic onium cation.

The second onium salt compound generates a weak acid that does not induce dissociation of the acid-dissociable group of the polymer (A) through exposure to light. That is, the second onium salt compound generates an acid having a pKa higher than that of the acid generated from the first onium salt compound or the radiation-sensitive acid generator through irradiation with radiation. Since the second onium salt compound has a carboxylic acid anion structure, the quenching ability is high, the proton capture of the acid generated from the first onium salt compound or the like can be efficiently promoted, and the acid diffusion controlling ability in the non-exposed part of the first onium salt compound is improved. Since the second onium salt compound has a carboxylic acid anion structure and the first onium salt compound has a carboxy group, the compatibility of the two types of onium salts is improved, and in particular, aggregation of the first onium salt compound can be eliminated to improve dispersibility. As a result, excellent pattern rectangularity, pattern circularity, LWR performance, and MEEF performance can be exhibited.

As the monovalent organic group having 2 to 40 carbon atoms represented by R¹ of the formula (2), a monovalent organic group corresponding to the (1+n)-valent organic group having 1 to 40 carbon atoms represented by A of the formula (1) can be suitably employed.

The monovalent organic group preferably includes at least one structure selected from the group consisting of a cyclic structure (hereinafter, a second cyclic structure), an ester bond, an alcoholic hydroxyl group, a structure having a carboxy group, and an ether bond.

Examples of the second cyclic structure include the same as the first cyclic structure, and among them, it is preferable to have an alicyclic hydrocarbon structure, an aromatic hydrocarbon group, or a structure obtained by combining these.

Examples of the anion structure in the formula (2) include an anion structure represented by the following formula. Among those shown below, as the structure having an iodine group-containing aromatic ring structure, a structure in which an iodine atom in formulas below is substituted by an atom or group other than an iodine atom such as a hydrogen atom or another substituent can also be suitably employed.

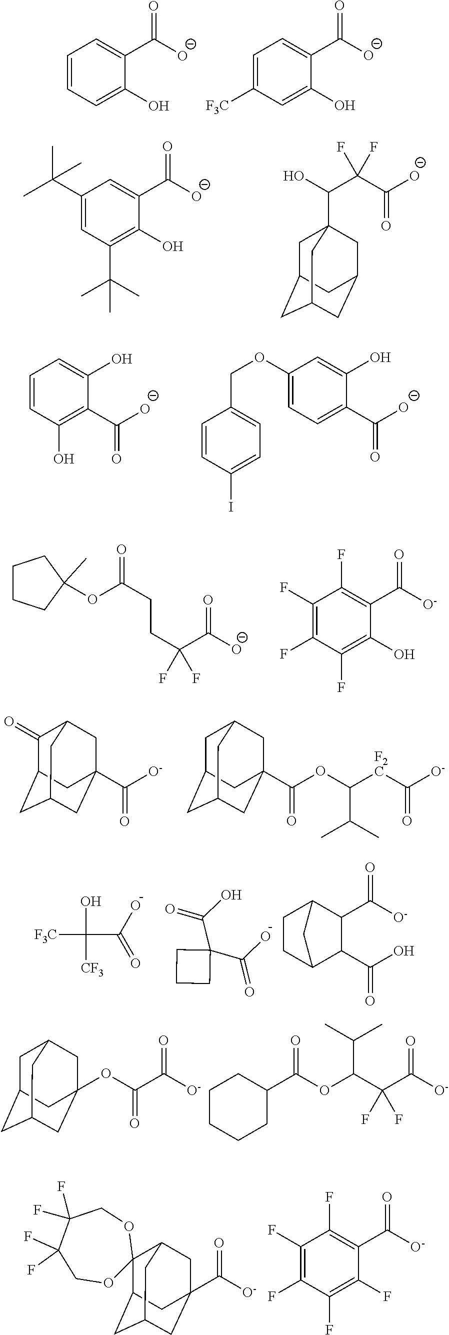

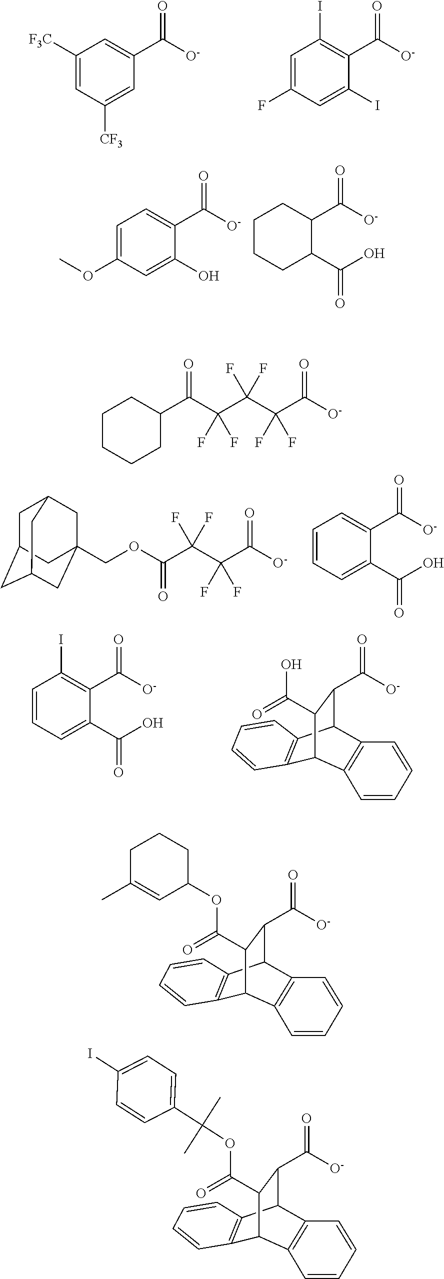

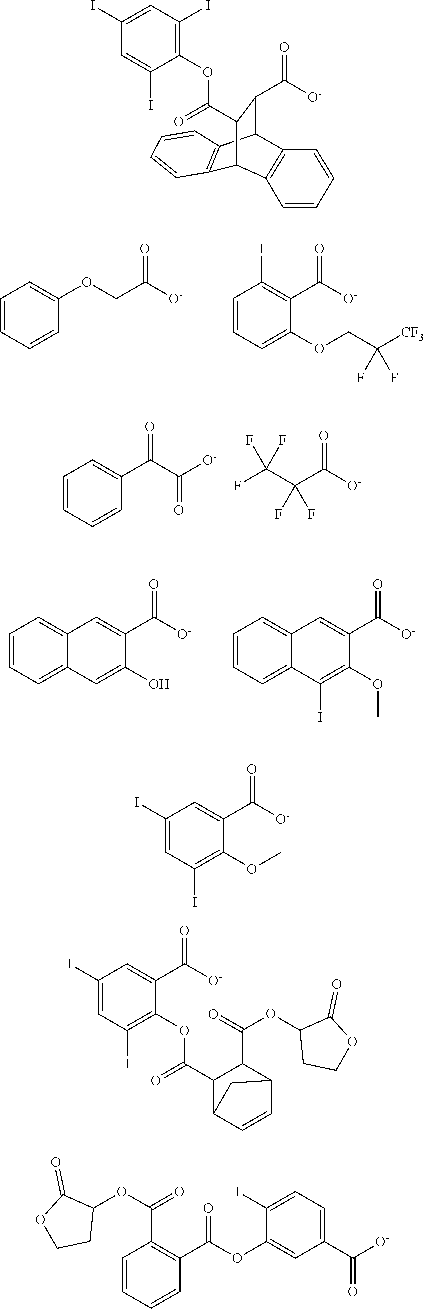

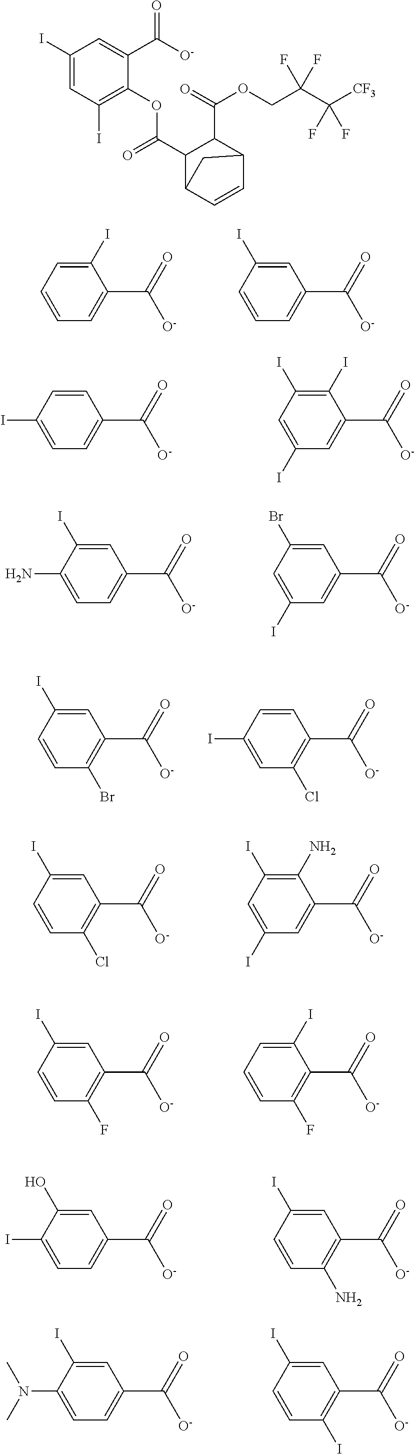

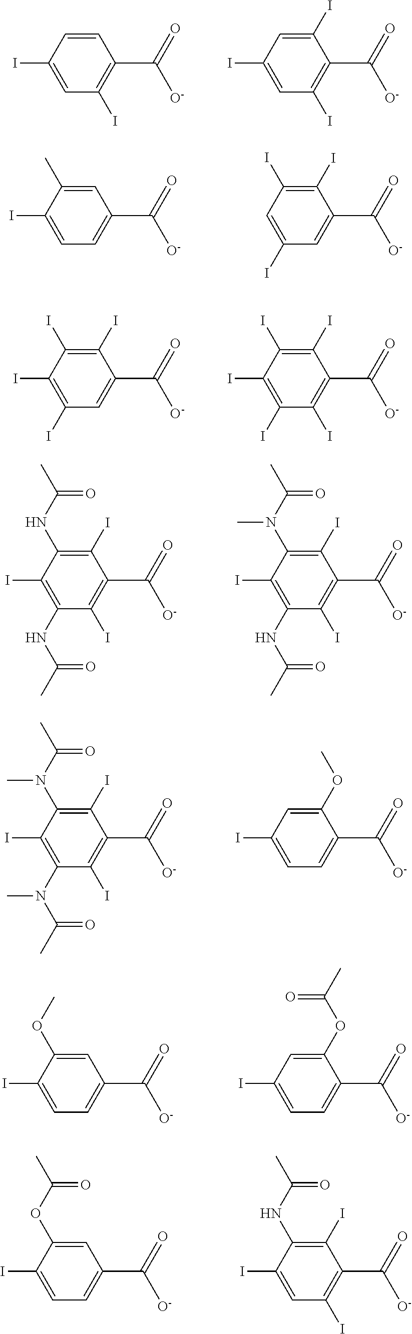

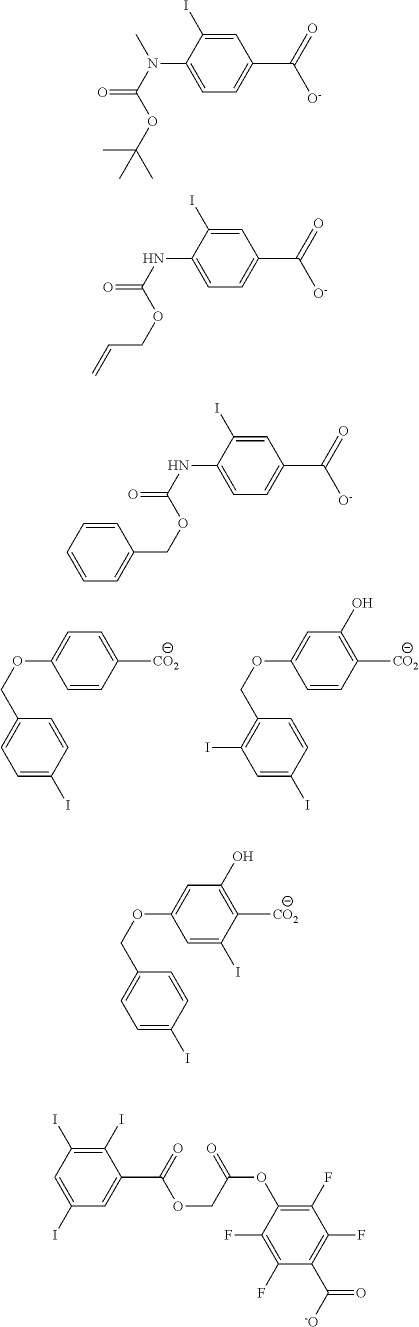

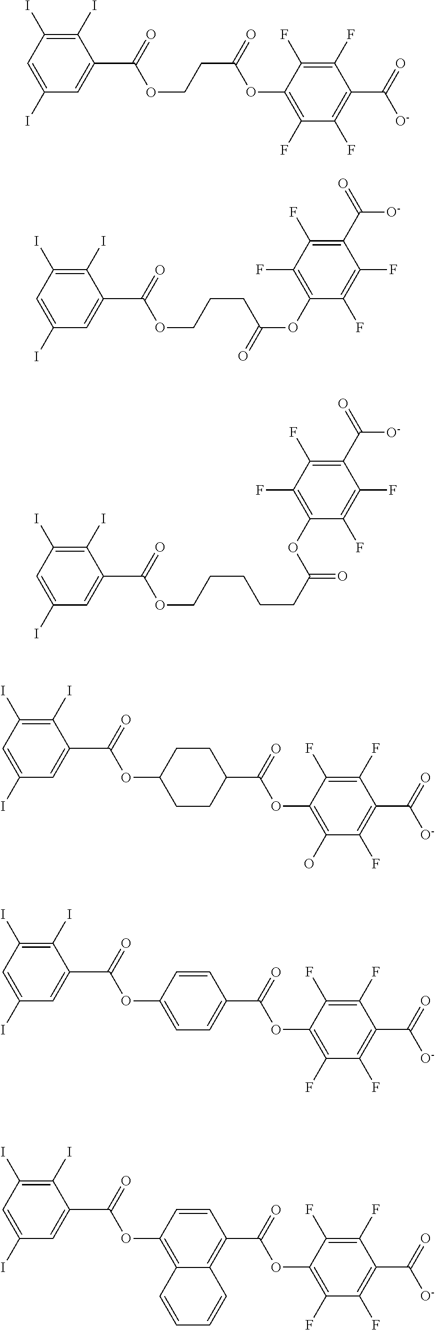 [SMILES: O=C(CCOC(=O)c1cc(I)cc(I)c1I)Oc1c(F)c(F)c(C(=O)[O-])c(F)c1F ; O=C(CCCOC(=O)c1cc(I)cc(I)c1I)Oc1c(F)c(F)c(C(=O)[O-])c(F)c1F ; O=C(CCCCCOC(=O)c1cc(I)cc(I)c1I)Oc1c(F)c(F)c(C(=O)[O-])c(F)c1F ; O=C(OC1CCC(C(=O)Oc2c(O)c(F)c(C(=O)[O-])c(F)c2F)CC1)c1cc(I)cc(I)c1I ; O=C(Oc1c(F)c(F)c(C(=O)[O-])c(F)c1F)c1ccc(OC(=O)c2cc(I)cc(I)c2I)cc1 ; O=C(Oc1ccc(C(=O)Oc2c(F)c(F)c(C(=O)[O-])c(F)c2F)c2ccccc12)c1cc(I)cc(I)c1I]

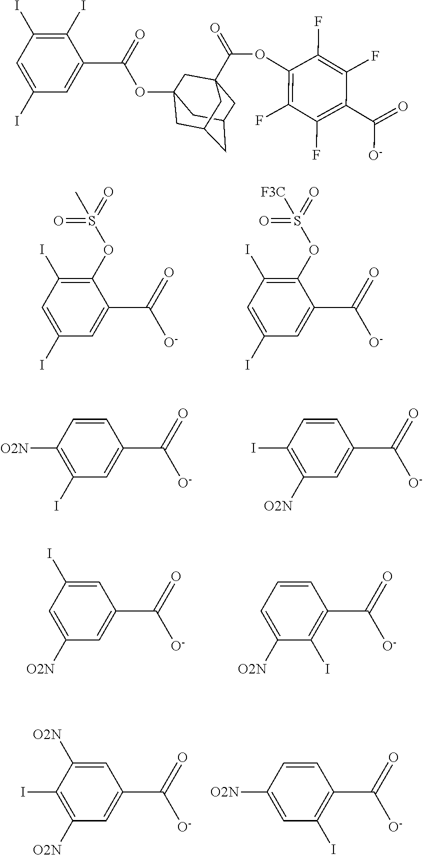

As the organic cation represented by Z₂⁺, the cation in the formula (1) can be suitably employed. Among them, a sulfonium cation is preferable.

As the formula (2), any combination of the anion structure and the onium cation may be included.

Specific examples of the carboxylic acid salt compound represented by formula (2) include, but are not limited to, structures represented by the formulas.

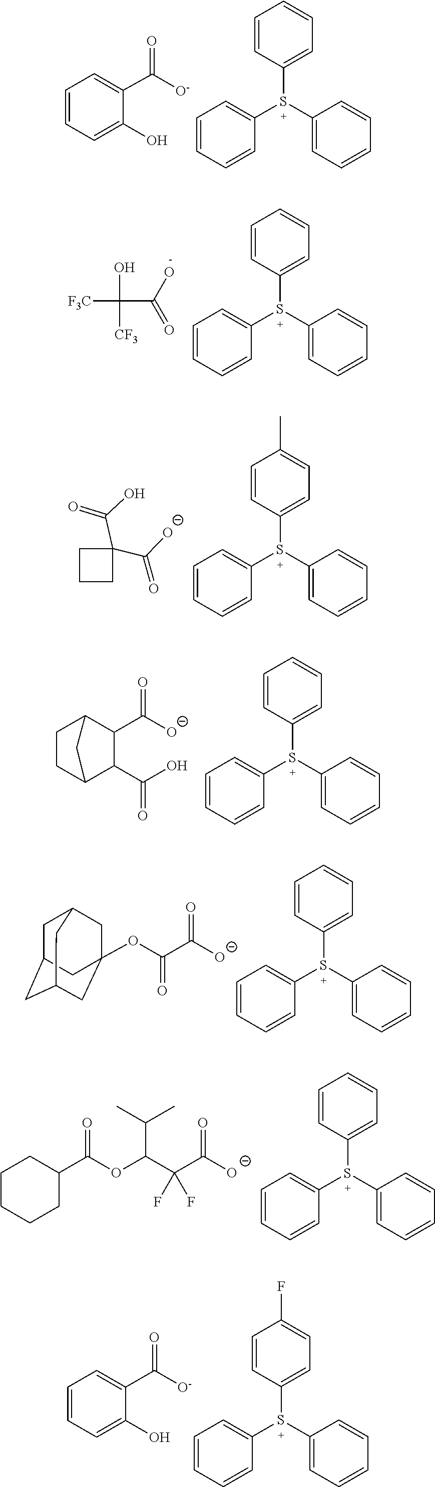

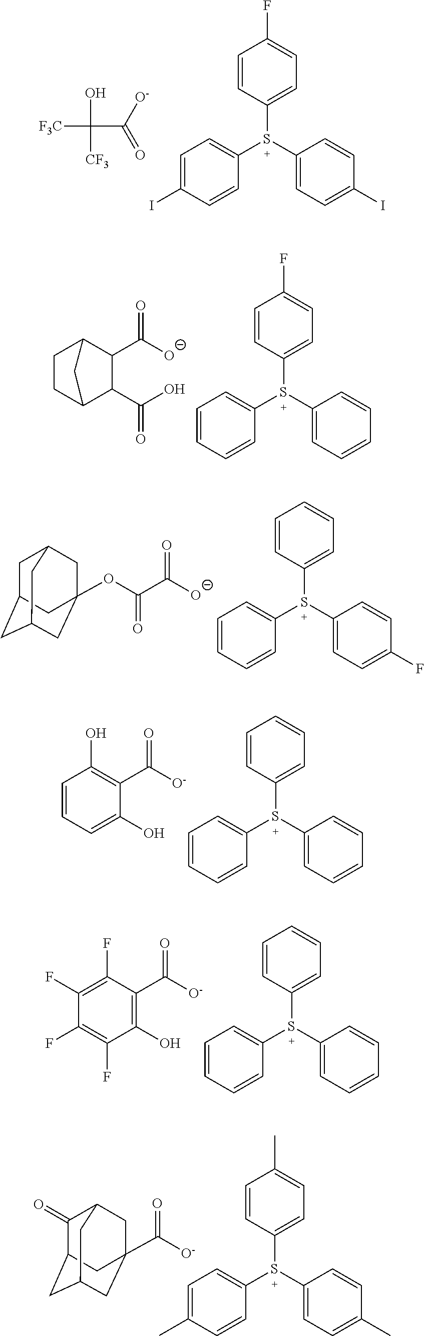

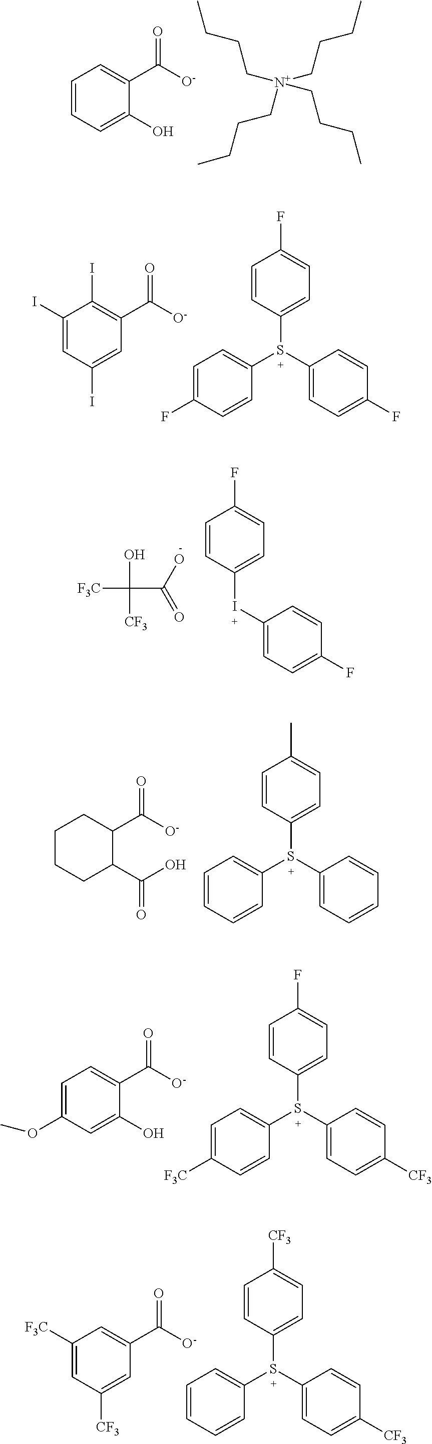

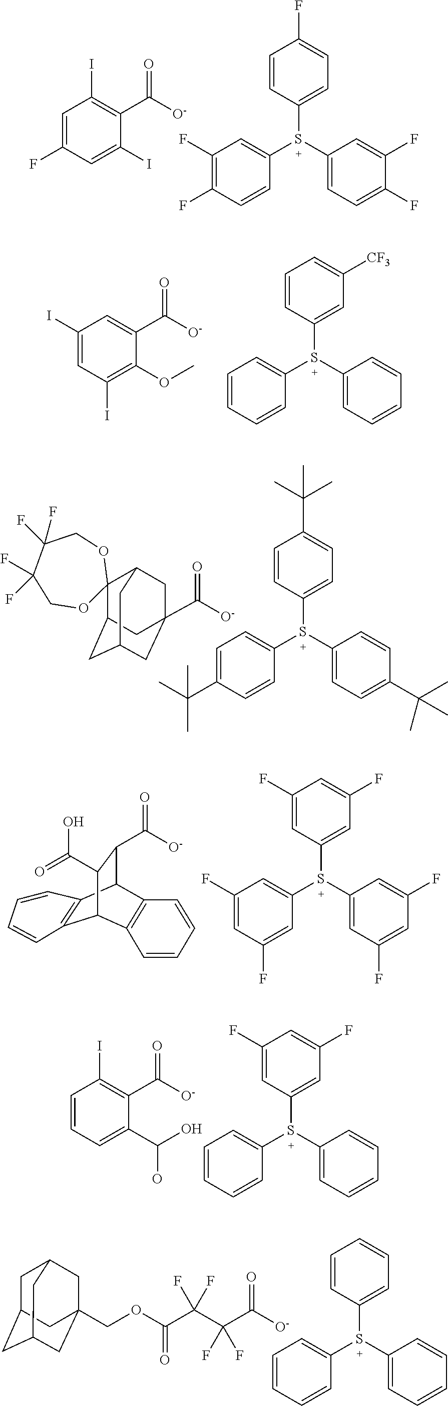

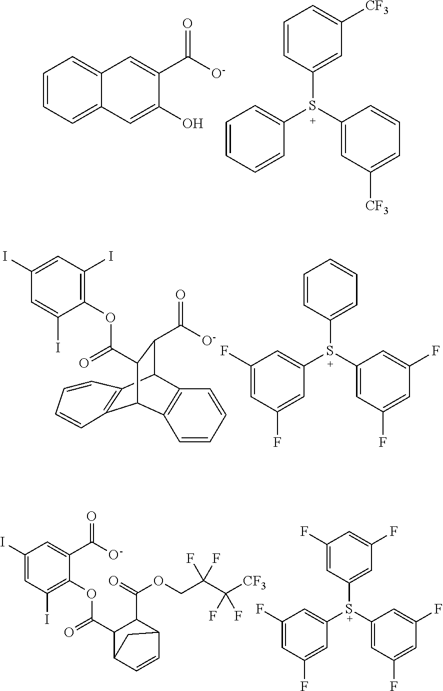

The carboxylic acid intramolecular salt compound represented by the formula (3) is a compound containing a cation and a carboxylic acid anion in the same molecule.

As the divalent organic group having 1 to 40 carbon atoms represented by R² of the formula (3), a divalent organic group corresponding to the (1+n)-valent organic group having 1 to 40 carbon atoms represented by A of the formula (1) can be suitably employed.

Z₃⁺ of the formula (3) is a monovalent organic onium cation, and examples thereof include a monovalent group having a monovalent sulfonium cation structure, and a monovalent group having an iodonium cation structure. Specific examples thereof include structures represented by *—R²¹—I⁺—R²² and *—R²³—S⁺—(R²⁴)₂. R²² and R²⁴ are each a monovalent organic group, and a monovalent organic group corresponding to the (1+n)-valent organic group having 1 to 40 carbon atoms represented by A of the formula (1) can be suitably employed. R²¹ and R²³ are each a divalent organic group, and a divalent organic group corresponding to the (1+n)-valent organic group having 1 to 40 carbon atoms represented by A of the formula (1) can be suitably employed. “*” represents a bond side with R².

Examples of the carboxylic acid intramolecular salt compound represented by the formula (3) include compounds represented by formula below (3-1).

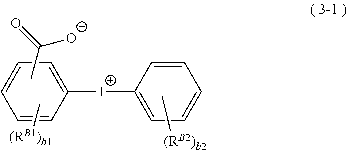

wherein, in formula (3-1), R^{B1} and R^{B2} are each independently a cyano group, a nitro group, an alkyl group, an alkoxy group, an alkoxycarbonyl group, an acyl group, or an acyloxy group; b1 represents an integer of 0 to 4, and when b1 is 2 or more, a plurality of R^{B1}s are the same as or different from each other; and b2 represents an integer of 0 to 4, and when b2 is 2 or more, a plurality of R^{B2}s are the same as or different from each other.

As the alkyl group, the alkoxycarbonyl group, the acyl group, and the acyloxy group, the alkyl group, the alkoxycarbonyl group, the acyl group, and the acyloxy group in R¹⁰² of the formula (5) can be suitably employed.

As the alkoxy group, the alkoxy group in X^{P1} of the formulas (4-1) to (4-7) can be suitably employed.

Specific examples of the carboxylic acid intramolecular salt compound represented by the formula (3) include the following compounds.

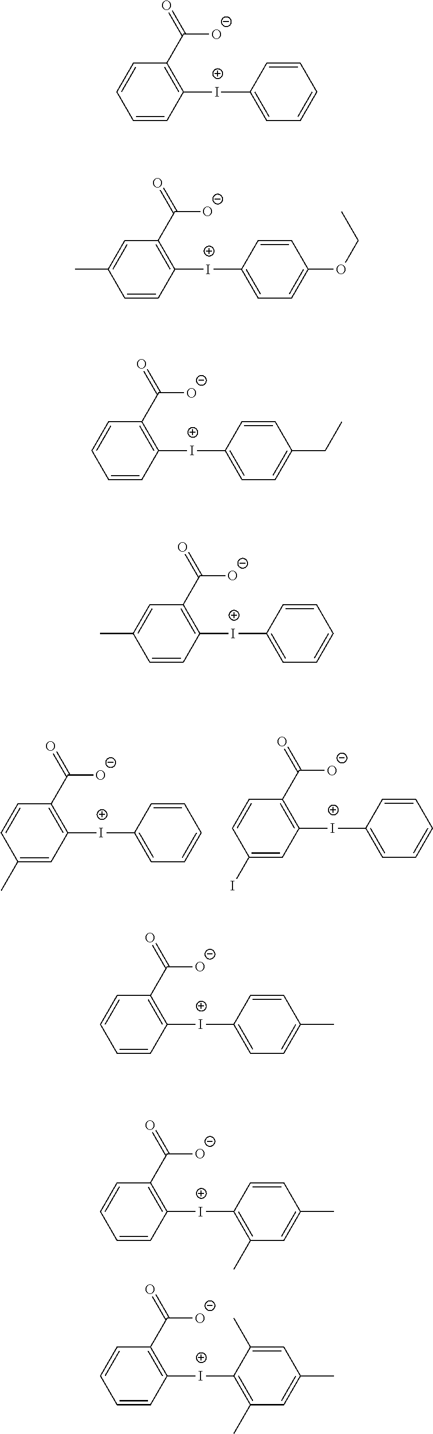

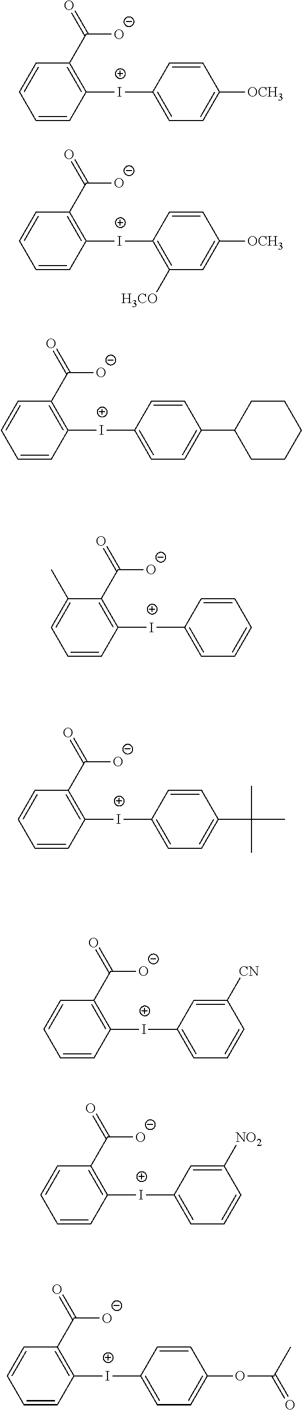

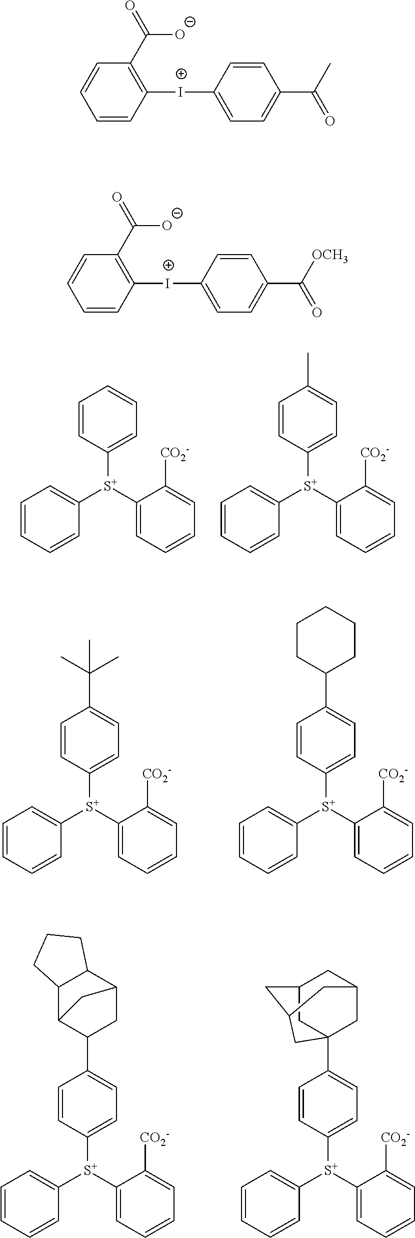

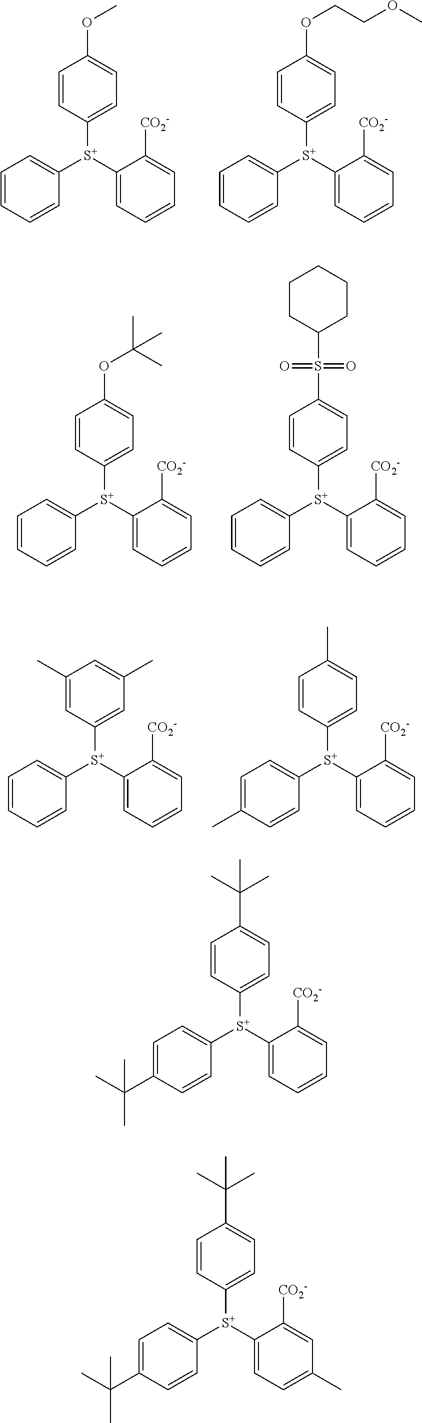

The second onium salt compound may be used singly, or two or more thereof may be used in combination. An acid diffusion controlling agent other than the second onium salt compound can be used in combination.

These second onium salt compounds may be used singly, or two or more thereof may be used in combination. The lower limit of the content of the second onium salt compound (the total content in the case of plural kinds of second onium salt compounds) is preferably 1 part by mass, more preferably 2 parts by mass, and still more preferably 3 parts by mass with respect to 100 parts by mass of the polymer (A). The upper limit of the content is preferably 30 parts by mass and more preferably 25 parts by mass with respect to 100 parts by mass of the polymer (A). This makes it possible to exhibit superior sensitivity and CDU performance when forming a resist pattern.

### (Another Polymer)

The radiation-sensitive composition according to the present embodiment may contain, as another polymer, a polymer having higher content by mass of fluorine atoms than the above-described base polymer (hereinafter, also referred to as a “high fluorine-content polymer”). When the radiation-sensitive composition contains the high fluorine-content polymer, the high fluorine-content polymer can be localized in the surface layer of a resist film compared to the base polymer, which as a result makes it possible to enhance the water repellency of the surface of the resist film during immersion exposure or to perform surface modification of the resist film during EUV exposure or control of the distribution of the composition in the film.

The high fluorine-content polymer preferably has, for example, a structural unit represented by the formula (5) (hereinafter, also referred to as “structural unit (V)”), and may have the structural unit (I) or the structural unit (III) in the base polymer as necessary.

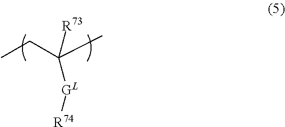

In the formula (5), R⁷³ is a hydrogen atom, a methyl group, or a trifluoromethyl group; GL is a single bond, an alkanediyl group having 1 to 5 carbon atoms, an oxygen atom, a sulfur atom, —COO—, —OCO—, —SO₂ONH—, —CONH—, —OCONH—, or a combination thereof; and R⁷⁴ is a monovalent fluorinated chain hydrocarbon group having 1 to 20 carbon atoms, or a monovalent fluorinated alicyclic hydrocarbon group having 3 to 20 carbon atoms.

As R⁷³ as described above, in terms of the copolymerizability of monomers resulting in the structural unit (V), a hydrogen atom or a methyl group is preferred, and a methyl group is more preferred.

As GL as described above, a combination of at least one of a single bond, —COO—, —OCO— and an alkanediyl group having 1 to 5 carbon atoms is preferable, and —COO— is more preferable from the viewpoint of the copolymerizability of a monomer that gives the structural unit (V).

Example of the monovalent fluorinated chain hydrocarbon group having 1 to 20 carbon atoms represented by R⁷⁴ as described above includes a group in which a part of or all of hydrogen atoms in the linear or branched chain alkyl group having 1 to 20 carbon atoms is/are substituted with a fluorine atom.

Example of the monovalent fluorinated alicyclic hydrocarbon group having 3 to 20 carbon atoms represented by R⁷⁴ as described above includes a group in which a part of or all of hydrogen atoms in the monocyclic or polycyclic hydrocarbon group having 3 to 20 carbon atoms is/are substituted with a fluorine atom.

The R⁷⁴ as described above is preferably a fluorinated chain hydrocarbon group, more preferably a fluorinated alkyl group, and further preferably 1,1,2,2,2-pentafluoroethyl group, 2,2,2-trifluoroethyl group, 2,2,3,3,3-pentafluoropropyl group, 1,1,1,3,3,3-hexafluoropropyl group and 5,5,5-trifluoro-1,1-diethylpentyl group.

When the high fluorine-content polymer has the structural unit (V), the lower limit of the content of the structural unit (V) is preferably 40 mol %, more preferably 50 mol, and still more preferably 55 mol % based on the total amount of all structural units constituting the high fluorine-content polymer. The upper limit of the content is preferably 90 mol %, more preferably 85 mol %, and still more preferably 80 mol %. When the content of the structural unit (V) is set to fall within the above range, the content by mass of fluorine atoms of the high fluorine-content polymer can more appropriately be adjusted to further promote the localization of the high fluorine-content polymer in the surface layer of a resist film, as a result, the water repellency of the resist film during immersion exposure can be further improved.

The high fluorine-content polymer may have a fluorine atom-containing structural unit represented by the formula (f-2) (hereinafter, also referred to as a “structural unit (VI)”) in addition to or in place of the structural unit (V). When the high fluorine-content polymer has the structural unit (VI), solubility in an alkaline developing solution is improved, and therefore generation of development defects can be prevented.

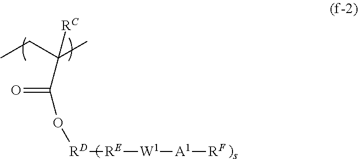

The structural unit (VI) is classified into two groups: a unit having an alkali soluble group (x); and a unit having a group (y) in which the solubility into the alkaline developing solution is increased by the dissociation by alkali (hereinafter, simply referred as an “alkali-dissociable group”). In both cases of (x) and (y), R^{C} in the formula (f-2) is a hydrogen atom, a fluorine atom, a methyl group, or a trifluoromethyl group; R^{D} is a single bond, a hydrocarbon group having 1 to 20 carbon atoms with the valency of (s+1), a structure in which an oxygen atom, a sulfur atom, —NR^{dd}—, a carbonyl group, —COO—, —OCO—, or —CONH— is connected to the terminal on R^{E} side of the hydrocarbon group, or a structure in which a part of hydrogen atoms in the hydrocarbon group is substituted with an organic group having a hetero atom; R^{dd} is a hydrogen atom, or a monovalent hydrocarbon group having 1 to 10 carbon atoms; and s is an integer of 1 to 3.

When the structural unit (VI) has the alkali soluble group (x), R^{F} is a hydrogen atom; A¹ is an oxygen atom, —COO—* or —SO₂O—*; * refers to a bond to R^{F}; W¹ is a single bond, a hydrocarbon group having 1 to 20 carbon atoms, or a divalent fluorinated hydrocarbon group. When A¹ is an oxygen atom, W¹ is a fluorinated hydrocarbon group having a fluorine atom or a fluoroalkyl group on the carbon atom connecting to A¹. R^{E} is a single bond, or a divalent organic group having 1 to 20 carbon atoms. When s is 2 or 3, a plurality of R^{E}, W¹, A¹ and R^{E} may be each identical or different. The affinity of the high fluorine-content polymer into the alkaline developing solution can be improved by including the structural unit (VI) having the alkali soluble group (x), and thereby prevent from generating the development defect. As the structural unit (VI) having the alkali soluble group (x), particularly preferred is a structural unit in which A¹ is an oxygen atom and W¹ is a 1,1,1,3,3,3-hexafluoro-2,2-methanediyl group.

When the structural unit (VI) has the alkali-dissociable group (y), R^{F} is a monovalent organic group having 1 to 30 carbon atoms; A¹ is an oxygen atom, —NR^{aa}—, —COO—*, —OCO—*, or —SO₂O—*; R^{aa} is a hydrogen atom, or a monovalent hydrocarbon group having 1 to 10 carbon atoms; * refers to a bond to R^{F}; W¹ is a single bond, or a divalent fluorinated hydrocarbon group having 1 to 20 carbon atoms; R^{E} is a single bond, or a divalent organic group having 1 to 20 carbon atoms. When A¹ is —COO—*, —OCO—* or —SO₂O—*, W¹ or R^{E} has a fluorine atom on the carbon atom connecting to A¹ or on the carbon atom adjacent to the carbon atom. When A¹ is an oxygen atom, W¹ and R^{E} are a single bond; RD is a structure in which a carbonyl group is connected at the terminal on R^{E} side of the hydrocarbon group having 1 to 20 carbon atoms; and R^{E} is an organic group having a fluorine atom. When s is 2 or 3, a plurality of RE, W¹, A¹ and R^{E} may be each identical or different. The surface of the resist film is changed from hydrophobic to hydrophilic in the alkaline developing step by including the structural unit (VI) having the alkali-dissociable group (y). As a result, the affinity of the high fluorine-content polymer into the alkaline developing solution can be significantly improved, and thereby prevent from generating the development defect more efficiently. As the structural unit (VI) having the alkali-dissociable group (y), particularly preferred is a structural unit in which A¹ is —COO—*, and R^{E} or W¹, or both is/are a group having a fluorine atom.

In terms of the copolymerizability of monomers resulting in the structural unit (VI), RC is preferably a hydrogen atom or a methyl group, and more preferably a methyl group.

When R^{E} is a divalent organic group, R^{E} is preferably a group having a lactone structure, more preferably a group having a polycyclic lactone structure, and further preferably a group having a norbornane lactone structure.

When the high fluorine-content polymer has the structural unit (VI), the lower limit of the content of the structural unit (VI) is preferably 40 mol %, more preferably 50 molo, and still more preferably 55 mol % based on the total amount of all structural units constituting the high fluorine-content polymer. The upper limit of the content is preferably 95 molo, more preferably 90 mol %, and still more preferably 85 mol %. When the content of the structural unit (VI) is set to fall within the above range, water repellency of a resist film during immersion exposure can further be improved, and development defects can be suppressed.

### [Other Structural Unit]

The high fluorine-content polymer may contain a structural unit having an alicyclic structure represented by the formula (6) in addition to the structural unit (I) and the structural unit (III) in the base polymer as a structural unit other than the structural units listed above.

When the high fluorine-content polymer contains the structural unit (I) and the structural unit (III), the content described for the base polymer can be suitably employed as the content of each structural unit in the high fluorine-content polymer.

When the high fluorine-content polymer contains the structural unit having an alicyclic structure, the lower limit of the content of the structural unit having an alicyclic structure is preferably 10 mol %, more preferably 20 mol %, and still more preferably 30 mol % based on all structural units constituting the high fluorine-content polymer. The upper limit of the content is preferably 60 mol %, more preferably 50 molo, and still more preferably 45 mol %.

The lower limit of the Mw of the high fluorine-content polymer is preferably 2,000, more preferably 3,000, still more preferably 4,000, and particularly preferably 5,000. The upper limit of the Mw is preferably 30,000, more preferably 20,000, still more preferably 10,000, particularly preferably 8,000.

The lower limit of the Mw/Mn of the high fluorine-content polymer is usually 1, and more preferably 1.1. In addition, the upper limit of the Mw/Mn is usually 5, preferably 3, and more preferably 2.

When the radiation-sensitive composition contains the high-fluorine-content polymer, the content of the high fluorine-containing polymer is preferably 0.5 parts by mass or more, more preferably 1 part by mass or more, still more preferably 1.5 parts by mass or more, and particularly preferably 2 parts by mass or more based on 100 parts by mass of the base polymer. The content of the high fluorine-containing polymer is preferably 15 parts by mass or less, more preferably 10 parts by mass or less, still more preferably 8 parts by mass or less, and particularly preferably 6 parts by mass or less.

When the content of the high fluorine-content polymer is set to fall within the above range, the high fluorine-content polymer can more effectively be localized in the surface layer of a resist film, which as a result makes it possible to further enhance the water repellency of the surface of the resist film during immersion exposure. The radiation-sensitive composition may contain one kind of high fluorine-content polymer or two or more kinds of high fluorine-content polymers.

### (Method for Synthesizing High Fluorine-Content Polymer)

The high fluorine-content polymer can be synthesized by a method similar to the above-described method for synthesizing a base polymer.

### (Solvent (E))

The radiation-sensitive composition according to the present embodiment contains a solvent. The solvent is not particularly limited as long as the solvent can dissolve or disperse at least a first onium salt compound, a second onium salt compound and a polymer, and a radiation-sensitive acid generator contained as desired, and the like.

Examples of the solvent include an alcohol-based solvent, an ether-based solvent, a ketone-based solvent, an amide-based solvent, an ester-based solvent, and a hydrocarbon-based solvent.

Examples of the alcohol-based solvent include:
a monoalcohol-based solvent having 1 to 18 carbon atoms, including iso-propanol, 4-methyl-2-pentanol, 3-methoxybutanol, n-hexanol, 2-ethylhexanol, furfuryl alcohol, cyclohexanol, 3,3,5-trimethylcyclohexanol, diacetone alcohol, and 1-methoxy-2-propanol;
a polyhydric alcohol having 2 to 18 carbon atoms, including ethylene glycol, 1,2-propylene glycol, 2-methyl-2,4-pentanediol, 2,5-hexanediol, diethylene glycol, dipropylene glycol, triethylene glycol, and tripropylene glycol; and
a partially etherized polyhydric alcohol-based solvent in which a part of hydroxy groups in the polyhydric alcohol-based solvent is etherized, including 3-methoxybutanol, and 1-methoxy-2-propanol (propylene glycol monomethyl ether).

In the present embodiment, alcohol acid ester-based solvents such as methyl lactate, ethyl lactate, propyl lactate, butyl lactate, methyl 2-hydroxyisobutyrate, i-propyl 2-hydroxyisobutyrate, i-butyl 2-hydroxyisobutyrate, and n-butyl 2-hydroxyisobutyrate are also included in the alcohol-based solvent.

Examples of the ether-based solvent include:
a dialkyl ether-based solvent, including diethyl ether, dipropyl ether, and dibutyl ether;
a cyclic ether-based solvent, including tetrahydrofuran and tetrahydropyran;
an ether-based solvent having an aromatic ring, including diphenylether and anisole (methyl phenyl ether); and
an etherized polyhydric alcohol-based solvent in which a hydroxy group in the polyhydric alcohol-based solvent is etherized.

Examples of the ketone-based solvent include:
a chain ketone-based solvent, including acetone, butanone, and methyl-iso-butyl ketone;
a cyclic ketone-based solvent, including cyclopentanone, cyclohexanone, and methylcyclohexanone; and 2,4-pentanedione, acetonylacetone, and acetophenone.

Examples of the amide-based solvent include:
a cyclic amide-based solvent, including N,N′-dimethyl imidazolidinone and N-methylpyrrolidone; and
a chain amide-based solvent, including N-methylformamide, N,N-dimethylformamide, N,N-diethylformamide, acetamide, N-methylacetamide, N,N-dimethylacetamide, and N-methylpropionamide.

Examples of the ester-based solvent include:
a monocarboxylate ester-based solvent, including n-butyl acetate;
a partially etherized polyhydric alcohol acetate-based solvent, including diethylene glycol mono-n-butyl ether acetate, propylene glycol monomethyl ether acetate, and dipropylene glycol monomethyl ether acetate;
a lactone-based solvent, including γ-butyrolactone and valerolactone;
a carbonate-based solvent, including diethyl carbonate, ethylene carbonate, and propylene carbonate; and
a polyhydric carboxylic acid diester-based solvent, including propylene glycol diacetate, methoxy triglycol acetate, diethyl oxalate, ethyl acetoacetate, and diethyl phthalate.

Examples of the hydrocarbon-based solvent include:
an aliphatic hydrocarbon-based solvent, including n-hexane, cyclohexane, and methylcyclohexane;
an aromatic hydrocarbon-based solvent, including benzene, toluene, di-iso-propylbenzene, and n-amylnaphthalene.

Among them, an alcohol-based solvent, an ester-based solvent, and an ether-based solvent are preferable, a monoalcohol-based solvent, a partially etherized polyhydric alcohol-based solvent, a lactone-based solvent, a monocarboxylate ester-based solvent, and a ketone-based solvent are more preferable, and propylene glycol monomethyl ether acetate, propylene glycol monomethyl ether acetate, γ-butyrolactone, ethyl lactate, cyclohexanone, propylene glycol monomethyl ether, diacetone alcohol, and methyl 2-hydroxyisobutyrate are still more preferable. The radiation-sensitive composition may include one type of the solvent, or two or more types of the solvents in combination.

### (Other Optional Components)

The radiation-sensitive composition may contain other optional components other than the above-descried components. Examples of other optional components include a cross-linking agent, a localization enhancing agent, a surfactant, an alicyclic backbone-containing compound, and a sensitizer. These other optional components may be used singly or in combination of two or more of them.

### <Method for Preparing Radiation-Sensitive Composition>

The radiation-sensitive composition can be prepared by, for example, mixing the first onium salt compound, the second onium salt compound, the polymer, and, as necessary, the high fluorine-content polymer or the like, as well as the solvent in a prescribed ratio. The radiation-sensitive composition is preferably filtered through, for example, a filter having a pore diameter of about 0.05 μm to 0.40 μm after mixing. The solid matter concentration of the radiation-sensitive composition is usually 0.1 mass % to 50 mass %, preferably 0.5 mass % to 30 mass %, more preferably 1 mass % to 20 mass %.

### <Method for Forming Pattern>

A pattern forming method according to an embodiment of the present disclosure includes:
a step (1) of applying the radiation-sensitive composition directly or indirectly on a substrate to form a resist film (hereinafter, also referred to as a “resist film forming step”);
a step (2) of exposing the resist film (hereinafter, also referred to as an “exposure step”); and
a step (3) of developing the exposed resist film (hereinafter, also referred to as a “developing step”).

In accordance with this method for forming a resist pattern, a high-quality resist pattern can be formed because of the use of the radiation-sensitive composition described above capable of forming a resist film superior in sensitivity in an exposure step, LWR performance, pattern rectangularity, CDU performance, pattern circularity, MEEF and DOF. Hereinbelow, each of the steps will be described.

### [Resist Film Forming Step]

In this step (the above mentioned step (1)), a resist film is formed with the radiation-sensitive composition. Examples of the substrate on which the resist film is formed include one traditionally known in the art, including a silicon wafer, silicon dioxide, and a wafer coated with aluminum. An organic or inorganic antireflection film may be formed on the substrate, as disclosed in JP-B-06-12452 and JP-A-59-93448. Examples of the applicating method include a rotary coating (spin coating), flow casting, and roll coating. After applicating, a prebake (PB) may be carried out in order to evaporate the solvent in the film, if needed. The temperature of PB is typically from 60° C. to 150° C., and preferably from 80° C. to 140° C. The duration of PB is typically from 5 seconds to 600 seconds, and preferably from 10 seconds to 300 seconds.

The lower limit of the thickness of the resist film to be formed is preferably 10 nm, more preferably 15 nm, and still more preferably 20 nm. The upper limit of the film thickness is preferably 500 nm, more preferably 400 nm, and still more preferably 300 nm. In particular, when a thick resist film is exposed to ArF excimer laser light in an exposure step described later, the lower limit of the film thickness may be 100 nm, may be 150 nm, or may be 200 nm.

When the immersion exposure is carried out, irrespective of presence of a water repellent polymer additive such as the high fluorine-content polymer in the radiation-sensitive composition, the formed resist film may have a protective film for the immersion which is not soluble into the immersion liquid on the film in order to prevent a direct contact between the immersion liquid and the resist film. As the protective film for the immersion, a solvent-removable protective film that is removed with a solvent before the developing step (for example, see JP-A-2006-227632); or a developer-removable protective film that is removed during the development of the developing step (for example, see WO2005-069076 and WO2006-035790) may be used. In terms of the throughput, the developer-removable protective film is preferably used.

When the next step, the exposure step, is performed with radiation having a wavelength of 50 nm or less, it is preferable to use a polymer having the structural unit (I) and the structural unit (IV) as the base polymer in the composition.

### [Exposing Step]

In this step (the above mentioned step (2)), the resist film formed in the resist film forming step as the step (1) is exposed by irradiating with a radioactive ray through a photomask (optionally through an immersion medium such as water). Examples of the radioactive ray used for the exposure include, depending on the desired line width of the pattern, visible ray, ultraviolet ray, far ultraviolet ray, extreme ultraviolet ray (EUV); an electromagnetic wave including X ray and γ ray; an electron beam; and a charged particle radiation such as α ray. Among them, far ultraviolet ray, an electron beam, or EUV is preferred. ArF excimer laser light (wavelength is 193 nm), KrF excimer laser light (wavelength is 248 nm), an electron beam, or EUV is more preferred. An electron beam or EUV having a wavelength of 50 nm or less which is identified as the next generation exposing technology is further preferred.

When the exposure is carried out by immersion exposure, examples of the immersion liquid include water and fluorine-based inert liquid. The immersion liquid is preferably a liquid which is transparent with respect to the exposing wavelength, and has a minimum temperature factor of the refractive index so that the distortion of the light image reflected on the film becomes minimum. However, when the exposing light source is ArF excimer laser light (wavelength is 193 nm), water is preferably used because of the ease of availability and ease of handling in addition to the above considerations. When water is used, a small proportion of an additive that decreases the surface tension of water and increases the surface activity may be added. Preferably, the additive cannot dissolve the resist film on the wafer and can neglect an influence on an optical coating at an under surface of a lens. The water used is preferably distilled water.

After the exposure, post exposure bake (PEB) is preferably carried out to promote the dissociation of the acid-dissociable group in the polymer by the acid generated from the radiation-sensitive acid generator with the exposure in the exposed part of the resist film. The difference of solubility into the developer between the exposed part and the non-exposed part is generated by the PEB. The temperature of PEB is typically from 50° C. to 180° C., and preferably from 80° C. to 130° C. The duration of PEB is typically from 5 seconds to 600 seconds, and preferably from 10 seconds to 300 seconds.

### [Developing Step]

In this step (the above mentioned step (3)), the resist film exposed in the exposing step as the step (2) is developed. By this step, the predetermined resist pattern can be formed. After the development, the resist pattern is washed with a rinse solution such as water or alcohol, and the dried, in general.

Examples of the developer used for the development include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, 1,5-diazabicyclo-[4.3.0]-5-nonene. Among them, an aqueous TMAH solution is preferred, and 2.38% by mass of aqueous TMAH solution is more preferred.

In the case of the development with organic solvent, examples of the solvent include an organic solvent, including a hydrocarbon-based solvent, an ether-based solvent, an ester-based solvent, a ketone-based solvent, and an alcohol-based solvent; and a solvent containing an organic solvent. Examples of the organic solvent include one, two or more solvents listed as the solvent for the radiation-sensitive composition. Among them, an ether-based solvent, an ester-based solvent or a ketone-based solvent is preferred. As the ether-based solvent, a glycol ether-based solvent is preferable, and ethylene glycol monomethyl ether and propylene glycol monomethyl ether are more preferable. The ester-based solvent is preferably an acetate ester-based solvent, and more preferably n-butyl acetate or amyl acetate. The ketone-based solvent is preferably a chain ketone, and more preferably 2-heptanone. The content of the organic solvent in the developer is preferably 80% by mass or more, more preferably 90% by mass or more, further preferably 95% by mass or more, and particularly preferably 99% by mass or more. Examples of the ingredient other than the organic solvent in the developer include water and silicone oil.

As described above, the developer may be either an alkaline developer or an organic solvent developer. The developer can be appropriately selected depending on whether the desired positive pattern or negative pattern is desired.

Examples of the developing method include a method of dipping the substrate in a tank filled with the developer for a given time (dip method); a method of developing by putting and leaving the developer on the surface of the substrate with the surface tension for a given time (paddle method); a method of spraying the developer on the surface of the substrate (spray method); and a method of injecting the developer while scanning an injection nozzle for the developer at a constant rate on the substrate rolling at a constant rate (dynamic dispense method).

### <Onium Salt Compound>

The onium salt compound according to the present embodiment is represented by the formula (1-a).

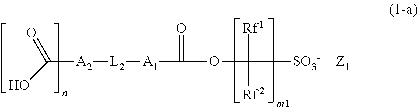

wherein, in the formula (1-a),
A₁ is a divalent organic group;
A₂ is a (1+n)-valent cyclic group;
L₂ is a divalent chain linking group containing an oxygen atom;
R^{f1} and R^{f2} are each independently a hydrogen atom, a monovalent organic group, a fluorine atom, or a monovalent fluorinated hydrocarbon group; when there are a plurality of R^{f1}s and R^{f2}s, the plurality of R^{f1}s and R^{f2}s are the same as or different from each other;
m1 is an integer of 1 to 8;
n is an integer of 1 to 3; and
Z₁⁺ represents a radiation-sensitive onium cation.

As the divalent organic group represented by A₁, a divalent organic group corresponding to the (1+n)-valent organic group having 1 to 40 carbon atoms represented by A of the formula (1) can be suitably employed. Among them, A₁ is preferably a divalent cyclic group.

Examples of the divalent cyclic group include groups obtained by removing two hydrogen atoms from the cyclic structure. The cyclic structure may be any of an alicyclic structure, an aromatic ring structure, and a heterocyclic structure, may be either a monocyclic structure or a polycyclic structure, and may be either saturated or unsaturated. Examples of the cyclic group include a group obtained by substituting a part or all of hydrogen atoms contained in a group having a cyclic structure by a substituent and a group containing, in a carbon-carbon bond (including both between two adjacent carbons and between two non-adjacent carbons) of such a group, a divalent heteroatom-containing group. As the alicyclic structure, the aromatic ring structure, and the heterocyclic structure, the alicyclic structure, the aromatic ring structure, and the heterocyclic structure in A of the formula (1) can be suitably employed.

A₂ is a (1+n)-valent cyclic group. As the cyclic group, a group obtained by removing (n-1) hydrogen atoms from the divalent cyclic group can be suitably employed. The carboxy group is preferably directly bonded to the cyclic structure.

Examples of the divalent chain linking group containing an oxygen atom represented by L₂ include —O—, —CO—, —CO—NH—, or a combination thereof, and a combination of —O—, —CO—, —CO—NH—, or a combination thereof with an alkanediyl group, and specific examples thereof include *—O—CO—, *—CO—O—, *—O—CO—O—, *—O—CO—CH₂—O—, *—CO—NH—, *—NH—CO—, and *—CO—CH₂—. “*” represents a bond with A₂. Among them, L₂ is preferably a divalent chain linking group containing an ester bond (*—O—CO—, *—CO—O—) or a carbonate bond (*—O—CO—O—).

R^{f1} and R^{f2} have the same meaning as in the formula (1). In particular, at least one of R^{f1} and R^{f2} is preferably a fluorine atom or a monovalent fluorinated hydrocarbon group, at least one of R^{f1} and R^{f2} on the carbon atom at the x position with respect to the sulfur atom of SO₃⁻ is more preferably a fluorine atom or a monovalent fluorinated hydrocarbon group, and both of R^{f1} and R^{f2} on the carbon atom at the x position with respect to the sulfur atom of SO₃⁻ are still more preferably a fluorine atom or a monovalent fluorinated hydrocarbon group.

m1, n, and Z₁⁺ have the same meaning as in the formula (1).

Examples of the anion structure of the onium salt compound represented by the formula (1-a) include those corresponding to the formula (1-a) among the examples of the anion structure of the first onium salt compound represented by the formula (1).

### EXAMPLES

Hereinafter, the present invention will be specifically described with reference to Examples, but the present invention is not limited to these Examples. Methods for measuring various physical property values are shown below.

### [Weight-Average Molecular Weight (Mw) and Number-Average Molecular Weight (Mn)]

The Mw and Mn of a polymer were measured under the conditions described above. A degree of dispersion (Mw/Mn) was calculated from results of the measured Mw and Mn.

### [¹³C-NMR Analysis]

¹³C-NMR analysis of the polymer was performed using a nuclear magnetic resonance apparatus (“JNM-Delta 400” manufactured by JEOL Ltd.).

### <Synthesis of Polymer>

Monomers used for synthesis of polymers in Examples and Comparative Examples are shown below. In the following synthesis examples, unless otherwise specified, “parts by mass” means a value taken when the total mass of the monomers used is 100 parts by mass, and “mol %” means a value taken when the total number of moles of the monomers used is 100 mol %.

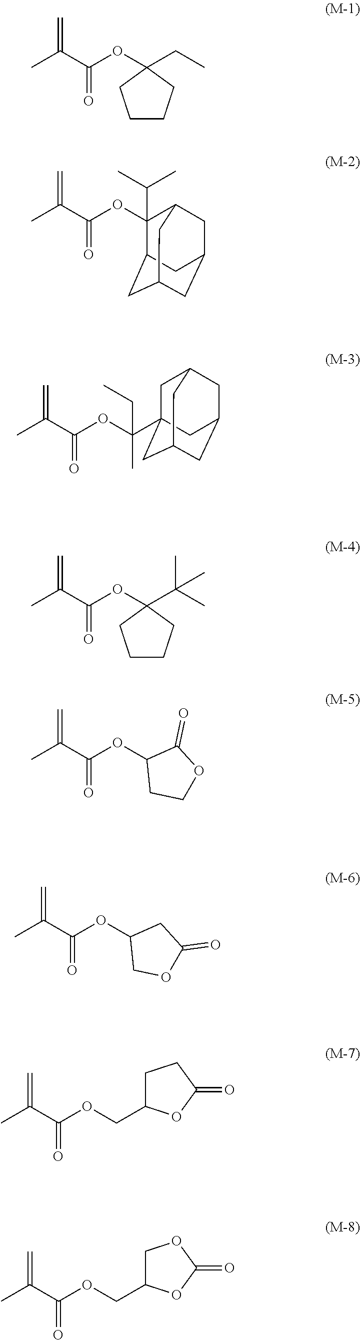

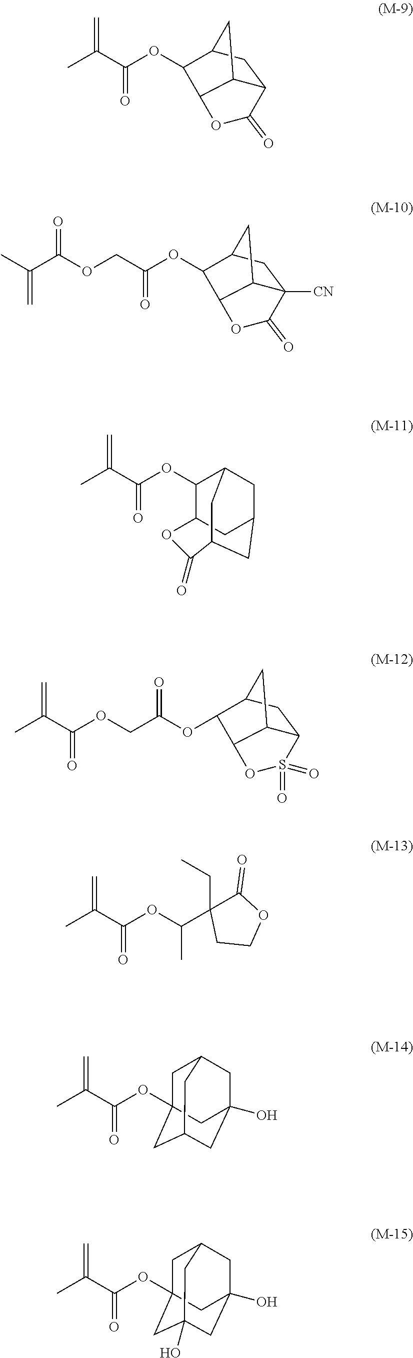

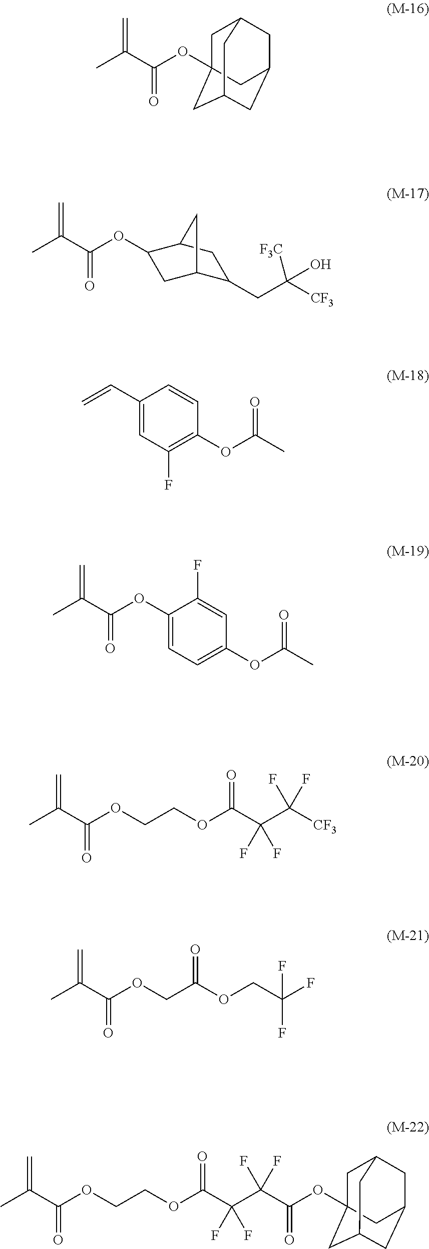

### Synthesis Example 1

### Synthesis of Polymer (A-1)

A monomer (M-1), a monomer (M-2), a monomer (M-5), a monomer (M-10), and a monomer (M-14) were dissolved at a molar ratio of 40/10/20/20/10 (mol %) in 2-butanone (200 parts by mass), and AIBN (azobisisobutyronitrile) (5 mol % based on 100 mol % in total of the monomers used) was added thereto as an initiator to prepare a monomer solution. 2-butanone (100 parts by mass) was placed in a reaction vessel, and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The polymerization solution cooled was poured into methanol (2,000 parts by mass), and a precipitated white powder was collected by filtration. The white powder separated by filtration was washed with methanol twice, then separated by filtration, and dried at 50° C. for 24 hours to obtain a white powdery polymer (A-1) (yield: 85%). The polymer (A-1) had an Mw of 7,100 and an Mw/Mn of 1.61. As a result of ¹³C-NMR analysis, the content ratio of the structural units derived from (M-1), (M-2), (M-5), (M-10), and (M-14) were 40.3 mol %, 9.2 mol %, 20.5 mol %, 19.8 mol %, and 10.2 mol %, respectively.

### Synthesis Examples 2 to 11

### Synthesis of Polymers (A-2) to (A-11)

Polymers (A-2) to (A-11) were synthesized in the same manner as in Synthesis Example 1 except that monomers of types and blending ratios shown in the following Table 1 were used. The content (mol %) of each of the structural units, yield (%), and physical property values (Mw and Mw/Mn) of the resulting polymers are also shown in Table 1. In Table 1, “-” indicates that the corresponding monomer was not used (the same applies to Tables below).

TABLE 1

| | | Monomer that affords structural | | | Monomer that affords structural | | | Monomer that affords structural | | | | |
|---|---|---|---|---|---|---|---|---|---|---|---|---|
| | | unit (I) | | | unit (II) | | | unit (III) and the like | | | | |
| | | | | Content ratio | | | Content ratio | | | Content ratio | | |
| | | | Blending | of structural | | Blending | of structural | | Blending | of structural | | |
| | Polymer | | ratio | unit | | ratio | unit | | ratio | unit | | |
| | (A) | Type | (mol %) | (mol %) | Type | (mol %) | (mol %) | Type | (mol %) | (mol %) | Mw | Mw/Mn |
| Synthesis | A-1 | M-1 | 40 | 40.3 | M-5 | 20 | 20.5 | M-14 | 10 | 10.2 | 7100 | 1.6 |
| Example 1 | | M-2 | 10 | 9.2 | M-10 | 20 | 19.8 | | | | | |
| Synthesis | A-2 | M-1 | 30 | 30.2 | M-9 | 50 | 50.6 | — | — | — | 7700 | 1.51 |
| Example 2 | | M-2 | 20 | 19.2 | | | | | | | | |
| Synthesis | A-3 | M-1 | 30 | 31.0 | M-11 | 50 | 49.4 | — | — | — | 7200 | 1.59 |
| Example 3 | | M-3 | 20 | 19.6 | | | | | | | | |
| Synthesis | A-4 | M-1 | 40 | 40.5 | M-12 | 50 | 49.2 | — | — | — | 6800 | 1.61 |
| Example 4 | | M-3 | 10 | 10.3 | | | | | | | | |
| Synthesis | A-5 | M-1 | 40 | 40.6 | M-13 | 50 | 51.3 | — | — | — | 6900 | 1.44 |
| Example 5 | | M-4 | 10 | 8.1 | | | | | | | | |
| Synthesis | A-6 | M-1 | 40 | 40.9 | M-6 | 20 | 20.5 | M-16 | 10 | 10.0 | 7500 | 1.51 |
| Example 6 | | M-4 | 10 | 8.3 | M-9 | 20 | 20.3 | | | | | |
| Synthesis | A-7 | M-1 | 50 | 50.2 | M-10 | 30 | 29.6 | M-14 | 20 | 20.2 | 7200 | 1.55 |
| Example 7 | | | | | | | | | | | | |
| Synthesis | A-8 | M-1 | 40 | 40.0 | M-7 | 20 | 20.5 | M-15 | 10 | 9.2 | 7100 | 1.62 |
| Example 8 | | M-3 | 10 | 10.1 | M-11 | 20 | 20.2 | | | | | |
| Synthesis | A-9 | M-1 | 50 | 50.3 | M-8 | 50 | 49.7 | — | — | — | 7000 | 1.51 |
| Example 9 | | | | | | | | | | | | |
| Synthesis | A-10 | M-1 | 40 | 40.2 | M-9 | 60 | 59.8 | — | — | — | 6700 | 1.50 |
| Example 10 | | | | | | | | | | | | |
| Synthesis | A-11 | M-2 | 40 | 39.4 | M-10 | 60 | 60.6 | — | — | — | 7500 | 1.49 |
| Example 11 | | | | | | | | | | | | |

### Synthesis Example 12

### Synthesis of Polymer (A-12)

Monomers (M-1) and (M-18) were dissolved at a molar ratio of 50/50 (mol %) in 1-methoxy-2 propanol (200 parts by mass), and AIBN (5 mol %) was added thereto as an initiator to prepare a monomer solution. 1-methoxy-2-propanol (100 parts by mass) was placed in a reaction vessel, and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The cooled polymerization solution was poured into hexane (2,000 parts by mass), and a precipitated white powder was collected by filtration. The white powder separated by filtration was washed with hexane twice, then separated by filtration, and dissolved in 1-methoxy-2-propanol (300 parts by mass). Next, methanol (500 parts by mass), triethylamine (50 parts by mass) and ultrapure water (10 parts by mass) were added, and a hydrolysis reaction was performed at 70° C. for 6 hours with stirring. After the completion of the reaction, the remaining solvent was distilled off. The resulting solid was dissolved in acetone (100 parts by mass), and the solution was added dropwise to water (500 parts by mass) to solidify a polymer. The resulting solid was separated by filtration, and dried at 50° C. for 13 hours to obtain a white powdery polymer (A-12) (yield: 81%). The polymer (A-12) had an Mw of 5, 500 and an Mw/Mn of 1.62. As a result of ¹³C-NMR analysis, the content ratio of the structural units derived from (M-1) and (M-18) were respectively 50.2 mol % and 49.8 mol %.

### Synthesis Examples 13 to 15

### Synthesis of Polymers (A-13) to (A-15)

Polymers (A-13) to (A-15) were synthesized in the same manner as in Synthesis Example 12 except that monomers of types and blending ratios shown in the following Table 2 were used. Note that, for the monomer that affords the structural unit (IV), in the polymer, the disappearance of the peak of the carbonyl group on the acetyl group was confirmed by measurement of ¹³C-NMR, and substantially all the alkali-dissociable groups were hydrolyzed to phenolic hydroxyl groups. The content ratio (mol %), yield (%), and physical property values (Mw and Mw/Mn) of each of the structural units of the resulting polymers are also shown in the following Table 2.

TABLE 2

| | | Monomer that affords structural | | | Monomer that affords structural | | | Monomer that affords structural | | | | |
|---|---|---|---|---|---|---|---|---|---|---|---|---|
| | | unit (I) | | | unit (III) | | | unit (IV) | | | | |
| | | | | Content ratio | | | Content ratio | | | Content ratio | | |
| | | | Blending | of structural | | Blending | of structural | | Blending | of structural | | |
| | Polymer | | ratio | unit | | ratio | unit | | ratio | unit | | |
| | (A) | Type | (mol %) | (mol %) | Type | (mol %) | (mol %) | Type | (mol %) | (mol %) | Mw | Mw/Mn |
| Synthesis | A-12 | M-1 | 50 | 50.2 | — | — | — | M-18 | 50 | 49.8 | 5500 | 1.62 |
| Example 12 | | | | | | | | | | | | |
| Synthesis | A-13 | M-3 | 50 | 46.6 | M-14 | 10 | 11.1 | M-19 | 40 | 42.3 | 5600 | 1.55 |
| Example 13 | | | | | | | | | | | | |
| Synthesis | A-14 | M-2 | 50 | 48.1 | M-17 | 20 | 21.3 | M-18 | 30 | 30.6 | 5100 | 1.59 |
| Example 14 | | | | | | | | | | | | |
| Synthesis | A-15 | M-4 | 55 | 53.2 | M-17 | 15 | 15.2 | M-19 | 30 | 31.6 | 6100 | 1.50 |
| Example 15 | | | | | | | | | | | | |

### Synthesis Example 16

### Synthesis of High Fluorine-Containing Polymer (F-1)

Monomers (M-1) and (M-20) were dissolved at a molar ratio of 20/80 (mol %) in 2-butanone (200 parts by mass), and AIBN (4 mol %) was added thereto as an initiator to prepare a monomer solution. 2-butanone (100 parts by mass) was placed in a reaction vessel, and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The solvent was replaced with acetonitrile (400 parts by mass). Hexane (100 parts by mass) was then added, followed by stirring, and an acetonitrile layer was collected. The operation was repeated three times. By replacing the solvent with propylene glycol monomethyl ether acetate, a solution of a high fluorine-containing polymer (F-1) was obtained (yield: 75%). The high fluorine-content polymer (F-1) had an Mw of 6, 200 and an Mw/Mn of 1.77. As a result of ¹³C-NMR analysis, the content ratio of the structural units derived from (M-1) and (M-20) were 19.5 mol % and 80.5 mol %, respectively.

### Synthesis Examples 17 to 20

### Synthesis of High Fluorine-Containing Polymers (F-2) to (F-5)

High fluorine-containing polymers (F-2) to (F-5) were synthesized in the same manner as in Synthesis Example 16 except that monomers of types and blending ratios shown in Table 3 were used. The content (mol %) of each of the structural units, yield (%), and physical property values (Mw and Mw/Mn) of the resulting high fluorine-containing polymers are also shown in Table 3.

TABLE 3

| | High | Monomer that affords | | | | | | | | | | | | | |
|---|---|---|---|---|---|---|---|---|---|---|---|---|---|---|---|
| | | structural unit | | | Monomer that affords | | | Monomer that affords | | | Monomer that affords | | | | |
| | fluo- | (V) or (VI) | | | structural unit(I) | | | structural unit(III) | | | other structural unit | | | | |
| | rine- | | Blen- | Content | | Blen- | Content | | Blen- | Content | | Blen- | Content | | |
| | content | | ding | ratio of | | ding | ratio of | | ding | ratio of | | ding | ratio of | | |
| | poly- | | ratio | structural | | ratio | structural | | ratio | structural | | ratio | structural | | |
| | mer | | (mol | unit | | (mol | unit | | (mol | unit | | (mol | unit | | Mw/ |
| | (F) | Type | %) | (mol %) | Type | %) | (mol %) | Type | %) | (mol %) | Type | %) | (mol %) | Mw | Mn |
| Synthesis | F-1 | M-20 | 80 | 80.5 | M-1 | 20 | 19.5 | — | — | — | — | — | — | 6200 | 1.77 |
| Example | | | | | | | | | | | | | | | |
| 16 | | | | | | | | | | | | | | | |
| Synthesis | F-2 | M-21 | 80 | 80.9 | M-4 | 20 | 19.1 | — | — | — | — | — | — | 7100 | 1.82 |
| Example | | | | | | | | | | | | | | | |
| 17 | | | | | | | | | | | | | | | |
| Synthesis | F-3 | M-22 | 60 | 62.3 | — | — | — | — | — | — | M-16 | 40 | 37.7 | 6900 | 1.91 |
| Example | | | | | | | | | | | | | | | |
| 18 | | | | | | | | | | | | | | | |
| Synthesis | F-4 | M-22 | 60 | 60.2 | M-2 | 20 | 19.4 | M-14 | 20 | 20.4 | — | — | — | 7300 | 1.88 |
| Example | | | | | | | | | | | | | | | |
| 19 | | | | | | | | | | | | | | | |
| Synthesis | F-5 | M-20 | 60 | 60.0 | M-3 | 10 | 10.1 | M-17 | 30 | 29.9 | — | — | — | 6700 | 1.87 |
| Example | | | | | | | | | | | | | | | |
| 20 | | | | | | | | | | | | | | | |

### Synthesis of Radiation-Sensitive Acid Generators (B)

### Synthesis Example 21

### Synthesis of compound (B-1)

A compound (B-1) was synthesized according to a synthesis scheme below.

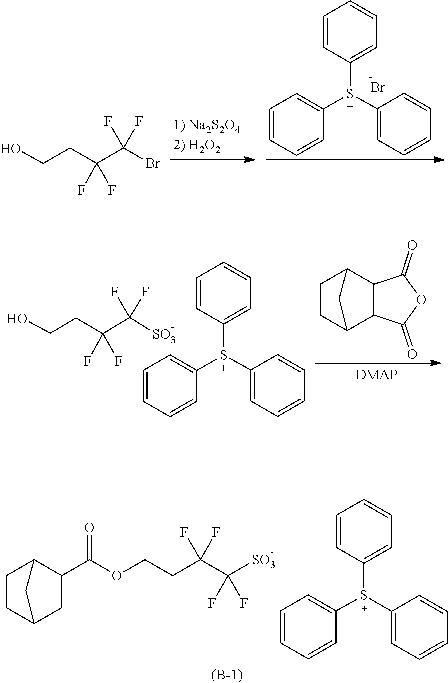

In a reaction vessel, a mixed liquid of acetonitrile and water (1:1 (mass ratio)) was added to 20.0 mmol of 4-bromo-3,3,4,4-tetrafluorobutan-1-ol to form a 1M solution. Then, 40.0 mmol of sodium dithionite and 60.0 mmol of sodium hydrogen carbonate were added, and the resulting mixture was reacted at 70° C. for 4 hours. After extraction with acetonitrile and distillation of the solvent, a mixed liquid of acetonitrile and water (3:1 (mass ratio)) was added to form a 0.5 M solution. To the solution were added 60.0 mmol of hydrogen peroxide and 2.00 mmol of sodium tungstate, followed by heating and stirring at 50° C. for 12 hours. The mixture was extracted with acetonitrile, and the solvent was distilled off to obtain a sodium sulfonate salt compound. 20.0 mmol of triphenylsulfonium bromide was added to the sodium sulfonate salt compound, and a mixed liquid of water and dichloromethane (1:3 (mass ratio)) was added to form a 0.5 M solution. The solution was vigorously stirred at room temperature for 3 hours. Thereafter, dichloromethane was added thereto, followed by extraction, and then the organic layer was separated. After drying the resulting organic layer over sodium sulfate, the solvent was distilled off, and purification was performed by column chromatography to obtain an onium salt form in a good yield.

20.0 mmol of norbornane-2,3-dicarboxylic anhydride, 5.00 mmol of 4-dimethylaminopyridine, and 50 g of dichloromethane were added to the onium salt form, and the mixture was stirred at room temperature for 13 hours. Thereafter, 1 M hydrochloric acid was added to stop the reaction, followed by addition of dichloromethane for extraction, and then the organic layer was separated. The resulting organic layer was washed with a saturated aqueous solution of sodium chloride and then with water. After drying over sodium sulfate, the solvent was distilled off, and purification was performed by column chromatography to obtain a compound (B-1) represented by the formula (B-1) in a good yield.

### Synthesis Examples 22 to 27

### Synthesis of Compounds (B-2) to (B-7)

Radiation-sensitive acid generators represented by the following formulas (B-2) to (B-7) were synthesized in the same manner as in Synthesis Example 21 except that the raw materials and the precursors were appropriately changed.

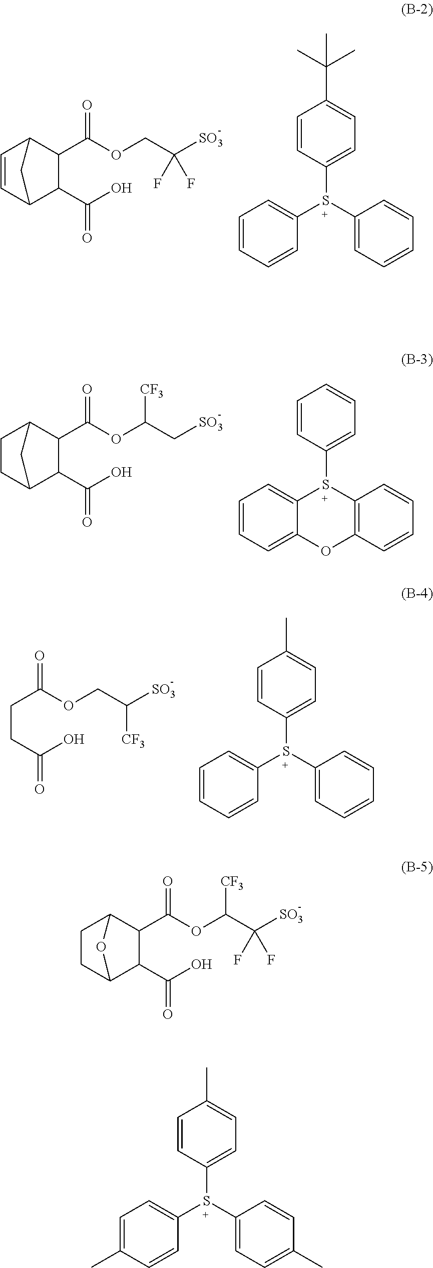

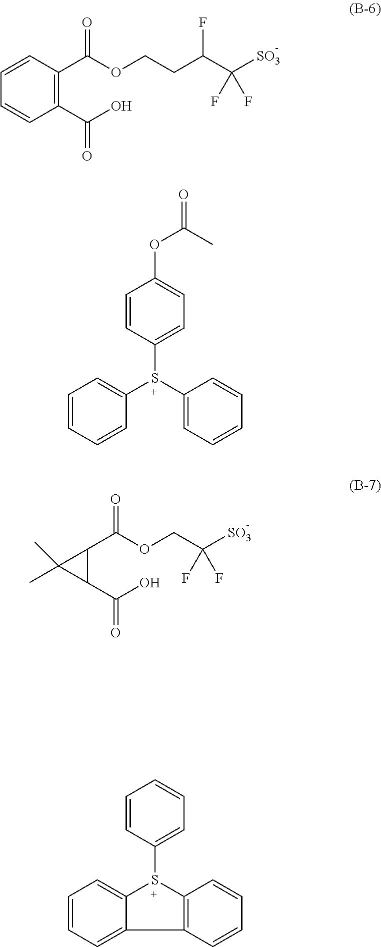

### Synthesis Example 28

### Synthesis of Compound (B-8)

A compound (B-8) was synthesized according to a 5 synthesis scheme below.

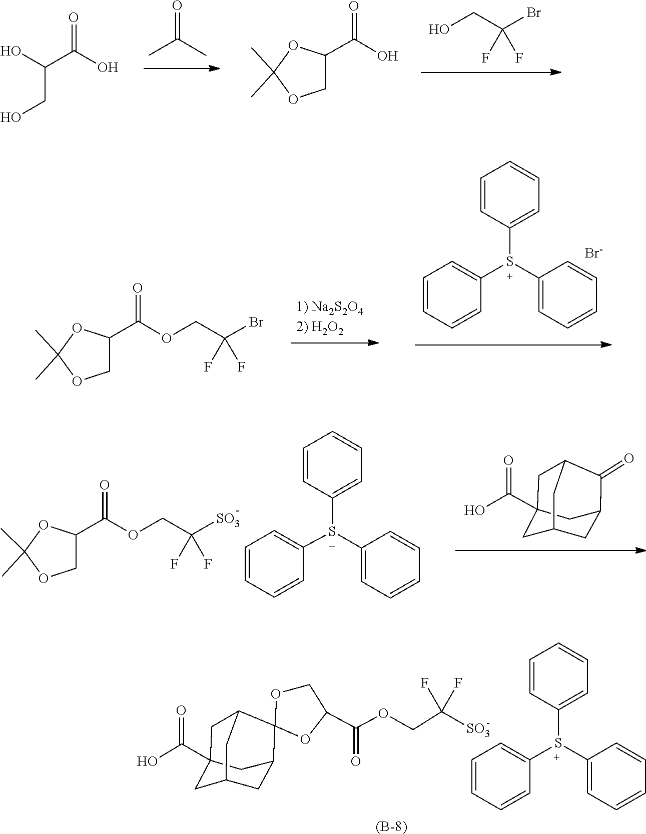

20.0 mmol of glyceric acid, 2.00 mmol of sulfuric acid, and 50 g of acetone were added to a reaction vessel, and the mixture was stirred stirring at room temperature for 2 hours. Thereafter, water was added to dilute the mixture, followed by addition of ethyl acetate and extraction, and then the organic layer was separated. The resulting organic layer was washed with a saturated aqueous solution of sodium chloride and then with water. After drying over sodium sulfate, the solvent was distilled off, and the residue was purified by column chromatography to obtain an acetal form in a good yield.

20.0 mmol of 2-bromo-2,2-difluoroethan-1-ol, 30.0 mmol of dicyclohexylcarbodiimide and 50 g of dichloromethane were added to the acetal form, and the mixture was stirred at room temperature for 4 hours. Thereafter, water was added to dilute the mixture, followed by addition of dichloromethane and extraction, and then the organic layer was separated. The resulting organic layer was washed with a saturated aqueous solution of sodium chloride and then with water. After drying over sodium sulfate, the solvent was distilled off, and the residue was purified by column chromatography to obtain an ester form in a good yield.

A mixed liquid of acetonitrile and water (1:1 (mass ratio)) was added to the ester form to form a 1 M solution. Then, 40.0 mmol of sodium dithionite and 60.0 mmol of sodium hydrogen carbonate were added, and the mixture was reacted at 70° C. for 4 hours. After extraction with acetonitrile and distillation of the solvent, a mixed liquid of acetonitrile and water (3:1 (mass ratio)) was added to form a 0.5 M solution. To the solution were added 60.0 mmol of hydrogen peroxide and 2.00 mmol of sodium tungstate, followed by heating and stirring at 50° C. for 12 hours. The mixture was extracted with acetonitrile, and the solvent was distilled off to obtain a sodium sulfonate salt compound. 20.0 mmol of triphenylsulfonium bromide was added to the sodium sulfonate salt compound, and a mixed liquid of water and dichloromethane (1:3 (mass ratio)) was added to form a 0.5 M solution. The solution was vigorously stirred at room temperature for 3 hours. Thereafter, dichloromethane was added thereto, followed by extraction, and then the organic layer was separated. After drying the resulting organic layer over sodium sulfate, the solvent was distilled off, and purification was performed by column chromatography to obtain an onium salt form in a good yield.

20.0 mmol of 2-adamantanone-5-carboxylic acid, 2.00 mmol of sulfuric acid and 50 g of dichloroethane were added to the onium salt form, and the mixture was stirred at 70° C. for 20 hours. Thereafter, water was added to dilute the mixture, followed by addition of dichloromethane and extraction, and then the organic layer was separated. The resulting organic layer was washed with a saturated aqueous solution of sodium chloride and then with water. After drying over sodium sulfate, the solvent was distilled off, and purification was performed by column chromatography to obtain a compound (B-8) represented by the formula (B-8) in a good yield.

### Synthesis Examples 29 to 34

### Synthesis of Compounds (B-9) to (B-14)

Onium salt compounds represented by the following formulas (B-9) to (B-14) were synthesized in the same manner as in Synthesis Example 28 except that the raw materials and the precursors were appropriately changed.

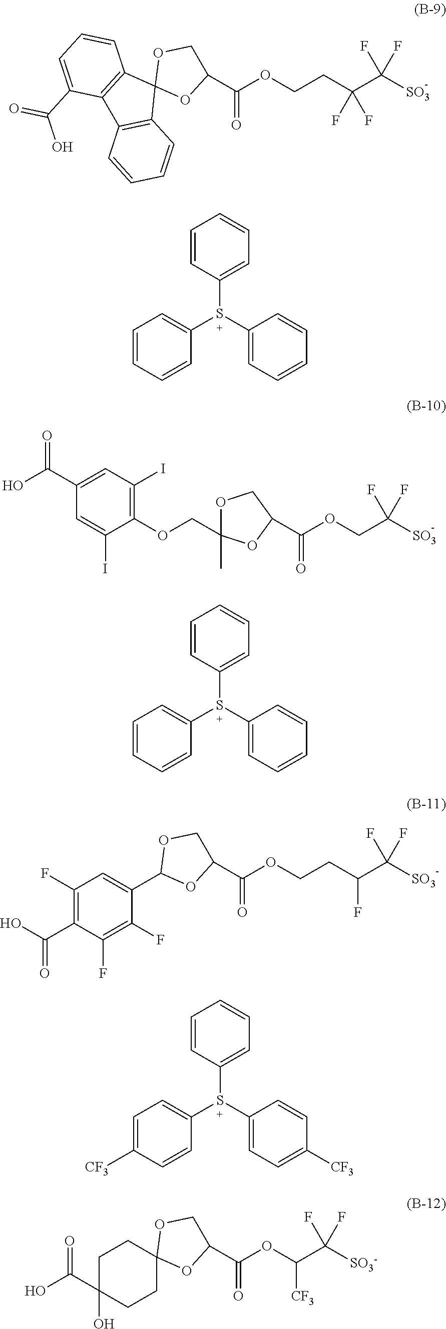

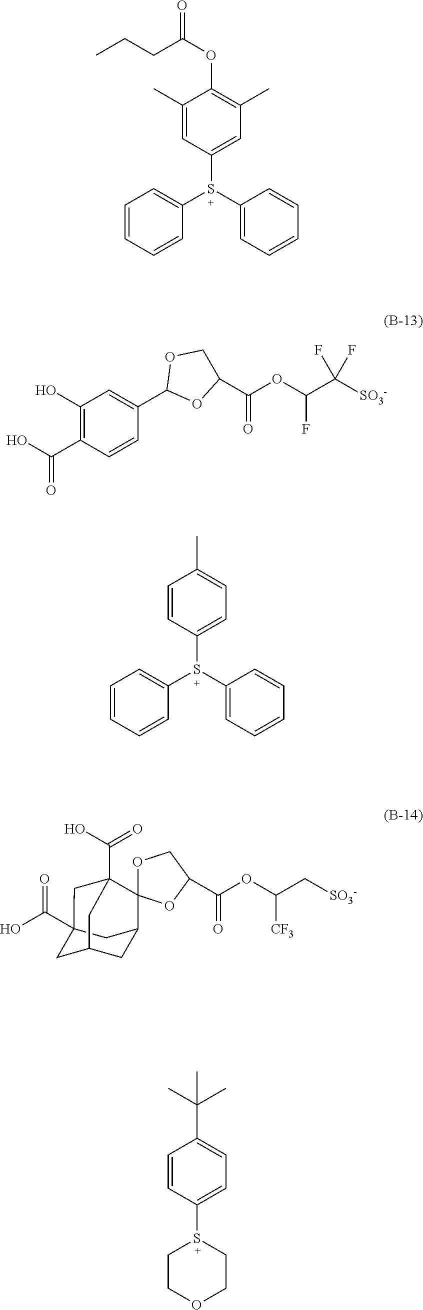

### Synthesis Example 35

### Synthesis of Compound (B-15)

A compound (B-15) was synthesized according to a synthesis scheme below.

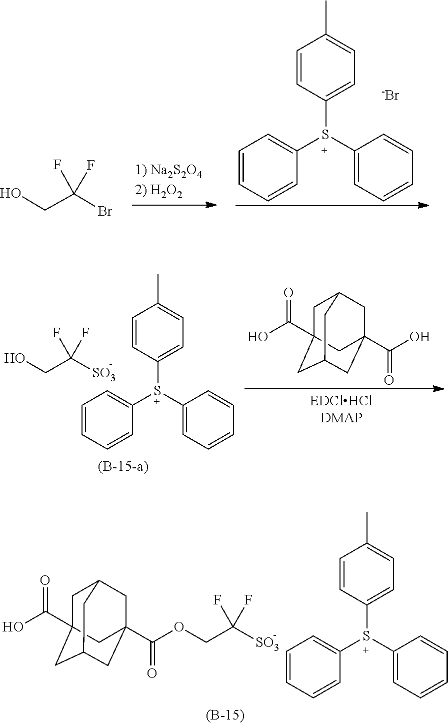

In a reaction vessel, a mixed liquid of acetonitrile and water (1:1 (mass ratio)) was added to 20.0 mmol of 2-bromo-2,2-difluoroethan-1-ol to form a 1M solution. Then, 40.0 mmol of sodium dithionite and 60.0 mmol of sodium hydrogen carbonate were added, and the resulting mixture was reacted at 70° C. for 4 hours. After extraction with acetonitrile and distillation of the solvent, a mixed liquid of acetonitrile and water (3:1 (mass ratio)) was added to form a 0.5 M solution. To the solution were added 60.0 mmol of hydrogen peroxide and 2.00 mmol of sodium tungstate, followed by heating and stirring at 50° C. for 12 hours. The mixture was extracted with acetonitrile, and the solvent was distilled off to obtain a sodium sulfonate salt compound. To the sodium sulfonate salt compound was added 20.0 mmol of diphenyl(p-tolyl) sulfonium bromide, and a mixed liquid of water and dichloromethane (1:3 (mass ratio)) was added thereto to form a 0.5 M solution. The solution was vigorously stirred at room temperature for 3 hours. Thereafter, dichloromethane was added thereto, followed by extraction, and then the organic layer was separated. After drying the resulting organic layer over sodium sulfate, the solvent was distilled off, and purification was performed by column chromatography to obtain an onium salt form (B-15-a) in a good yield.

20.0 mmol of 1,3-adamantane dicarboxylic acid, 25.0 mmol of 1-(3-dimethylaminopropyl)-3-ethylcarbodiimide hydrochloride, 5.0 mmol of 4-dimethylaminopyridine, and 50 g of dichloromethane were added to the onium salt form, and the mixture was reacted at room temperature for 12 hours. Thereafter, a 1 M aqueous hydrochloric acid solution was added to stop the reaction, followed by addition of dichloromethane for extraction, and then the organic layer was separated. After drying over sodium sulfate, the solvent was distilled off, and purification was performed by column chromatography to obtain a compound (B-15) represented by the formula (B-15) in a good yield.

### Synthesis Examples 36 to 40

### Synthesis of Compounds (B-16) to (B-20)

Radiation-sensitive acid generators represented by the following formulas (B-16) to (B-20) were synthesized in the same manner as in Synthesis Example 35 except that the raw materials and the precursors were appropriately changed.

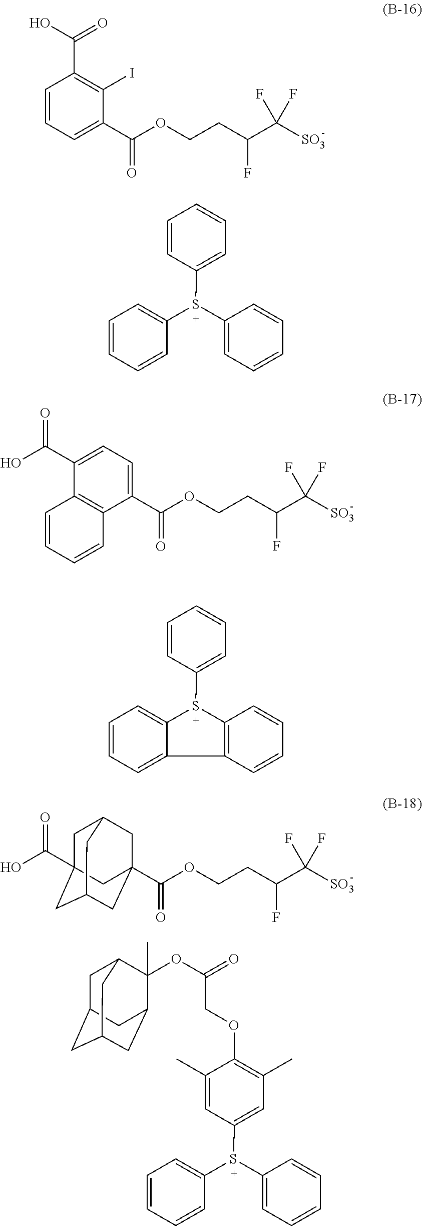

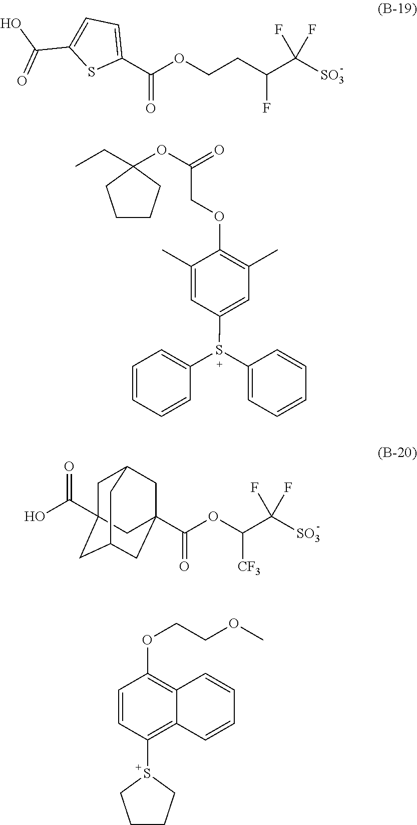

### Synthesis Example B1

### Synthesis of Compound (B-21)

A compound (B-21) was synthesized according to a synthesis scheme below.

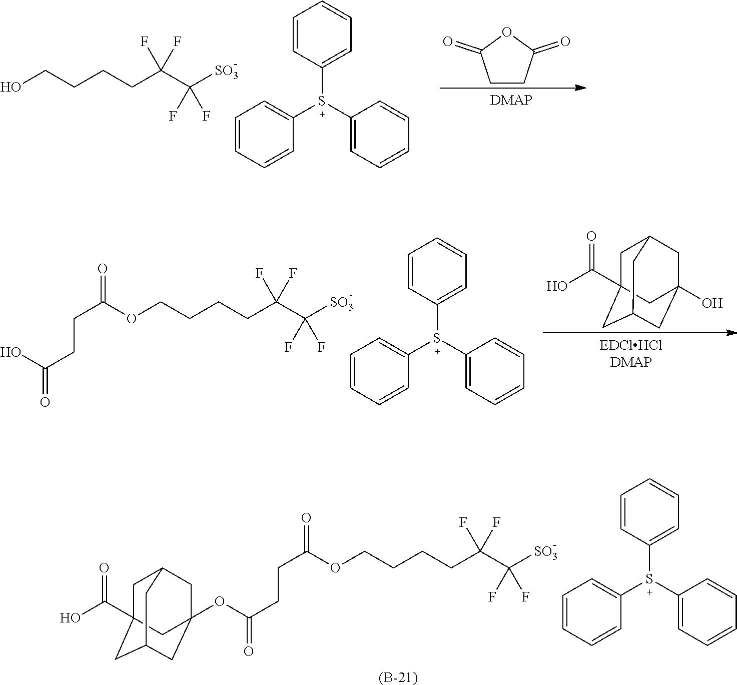

20.0 mmol of triphenylsulfonium 1,1,2,2-tetrafluoro-6-hydroxy-1-hexanesulfonate, 20.0 mmol of succinic anhydride, 5.0 mmol of 4-dimethylaminopyridine, and 50 g of dichloromethane were added to a reaction vessel, and the mixture was stirred at room temperature for 2 hours. Thereafter, a 1 M aqueous hydrochloric acid solution was added to stop the reaction, followed by addition of dichloromethane for extraction, and then the organic layer was separated. The resulting organic layer was washed with a saturated aqueous solution of sodium chloride. After drying over sodium sulfate, the solvent was distilled off, and purification was performed by column chromatography to obtain a carboxylic acid form in a good yield.

20.0 mmol of 3-hydroxy-1-adamantane carboxylic acid, 25.0 mmol of 1-(3-dimethylaminopropyl)-3-ethylcarbodiimide hydrochloride, 5.0 mmol of 4-dimethylaminopyridine, and 50 g of dichloromethane were added to the carboxylic acid form, and the mixture was reacted at room temperature for 12 hours. Thereafter, a 1 M aqueous hydrochloric acid solution was added to stop the reaction, followed by addition of dichloromethane for extraction, and then the organic layer was separated. After drying over sodium sulfate, the solvent was distilled off, and purification was performed by column chromatography to obtain a compound (B-21) represented by the formula (B-21) in a good yield.

### Synthesis Examples B2 to B7

### Synthesis of Compounds (B-22) to (B-27)

Radiation-sensitive acid generators represented by the following formulas (B-22) to (B-27) were synthesized in the same manner as in Synthesis Example B1 except that the raw materials and the precursors were appropriately changed.

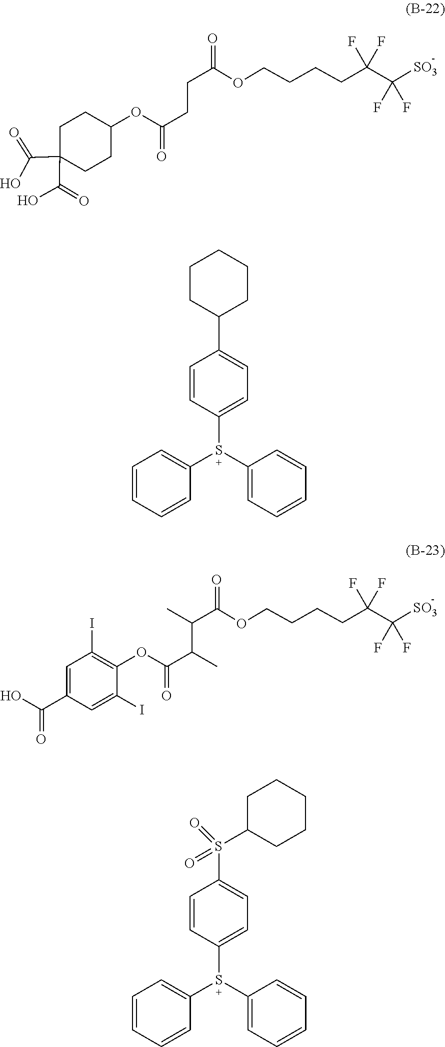

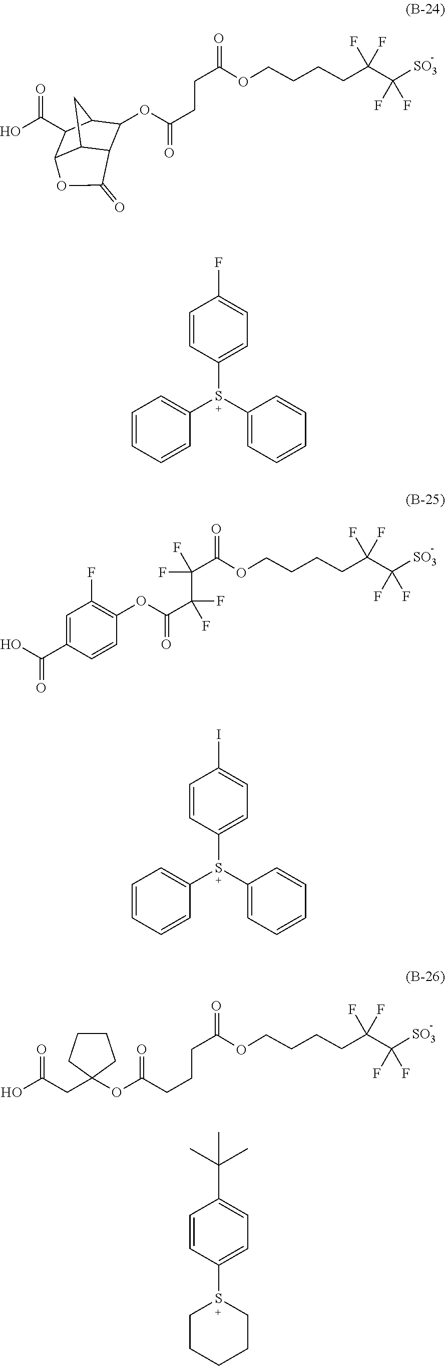

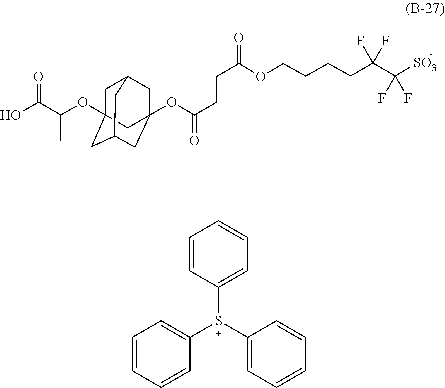

### Synthesis Example B8

### Synthesis of Compound (B-28)

A compound (B-28) was synthesized according to a synthesis scheme below.

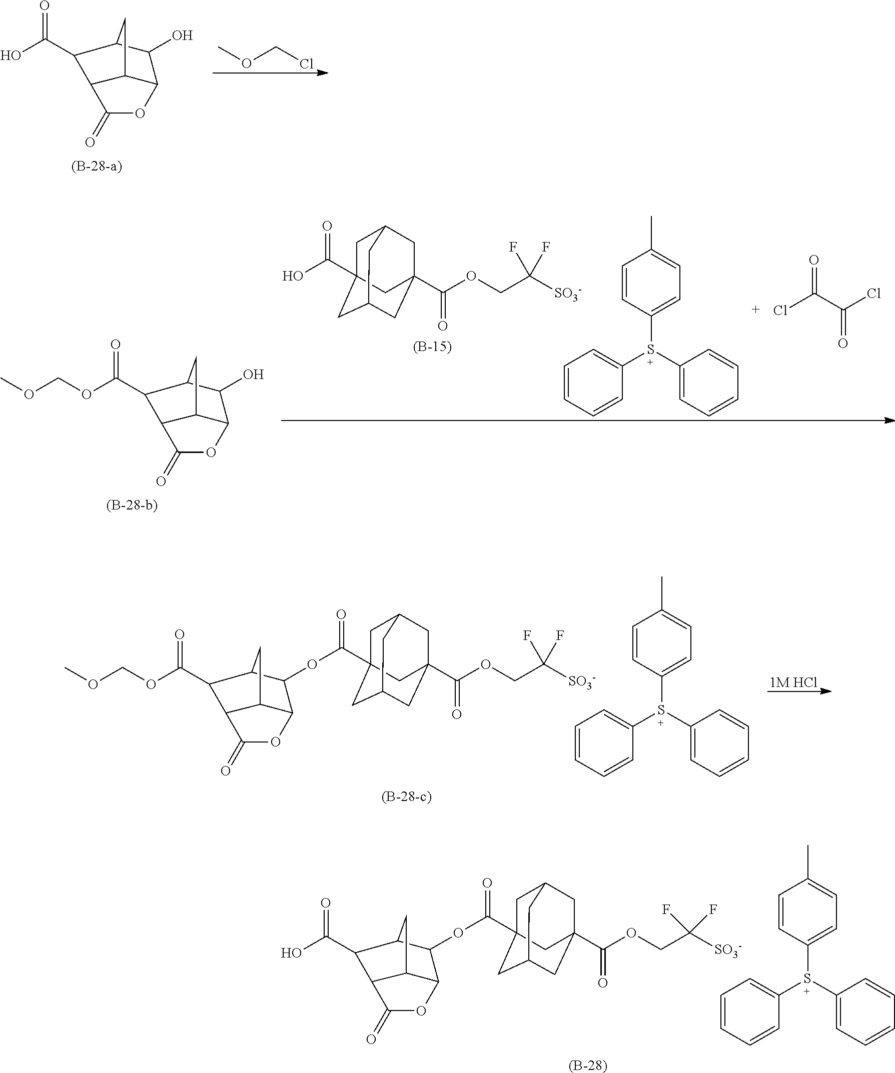

20.0 mmol of a compound (B-28-a), 20.0 mmol of chloromethyl methyl ether, 20.0 mmol of triethylamine, and 50 g of dichloromethane were added to a reaction vessel, and the mixture was stirred at room temperature for 13 hours. Thereafter, water was added to dilute the mixture, followed by addition of dichloromethane and extraction, and then the organic layer was separated. The resulting organic layer was washed with a saturated aqueous solution of sodium chloride and then with water. After drying over sodium sulfate, the solvent was distilled off, and the residue was purified by column chromatography to obtain a compound (B-28-b) in a good yield.

20.0 mmol of the onium salt compound (B-15) represented by the formula (B-15), 20.0 mmol of oxalyl chloride, and 50 g of acetonitrile were added to a reaction vessel, and the mixture was stirred at 50° C. for 2 hours. Then, 30.0 mmol of the compound (B-28-b), 30.0 mmol of pyridine, and 3.0 mmol of 4-dimethylaminopyridine were added, and the mixture was stirred at 100° C. for 10 hours. Thereafter, water was added to stop the reaction, dichloromethane was then added thereto to perform extraction, and the organic layer was separated. The resulting organic layer was washed with a saturated aqueous solution of sodium chloride and then with water. After drying over sodium sulfate, the solvent was distilled off, and the residue was purified by column chromatography to obtain an onium salt compound (B-28-c) in a good yield.

To the onium salt compound (B-28-c) represented by the formula (B-28-c), 50 g of 1-M hydrochloric acid and 50 g of acetonitrile were added, and the mixture was stirred at 100° C. for 3 hours. Thereafter, water was added to dilute the mixture, followed by addition of dichloromethane and extraction, and then the organic layer was separated. The resulting organic layer was washed with a saturated aqueous solution of sodium chloride and then with water. After drying over sodium sulfate, the solvent was distilled off, and purification was performed by column chromatography to obtain an onium salt compound (B-28) represented by the formula (B-28) in a good yield.

### Synthesis Examples B9 to B12

### Synthesis of Onium Salt Compounds (B-29) to (B-32)

Onium salt compounds (B-29) to (B-32) represented by the following formulas (B-29) to (B-32) were synthesized in the same manner as in Synthesis Example B8 except that the raw materials and the precursors were appropriately changed.

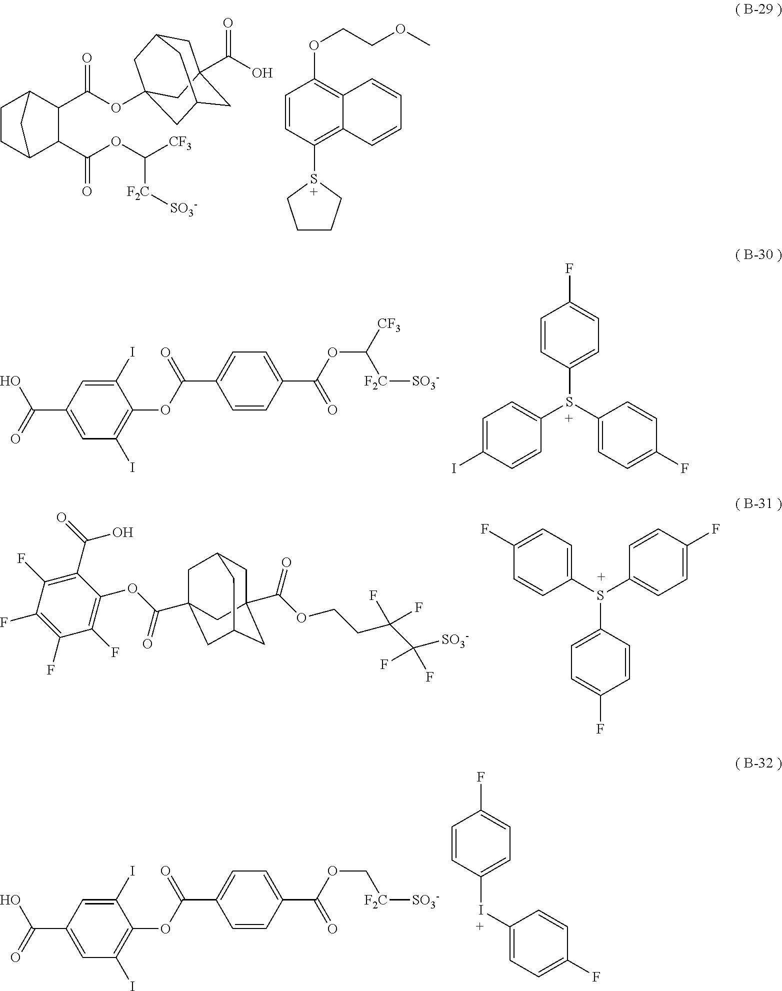

### Synthesis Example B13

### Synthesis of Compound (B-33)

A compound (B-33) was synthesized according to a synthesis scheme below.

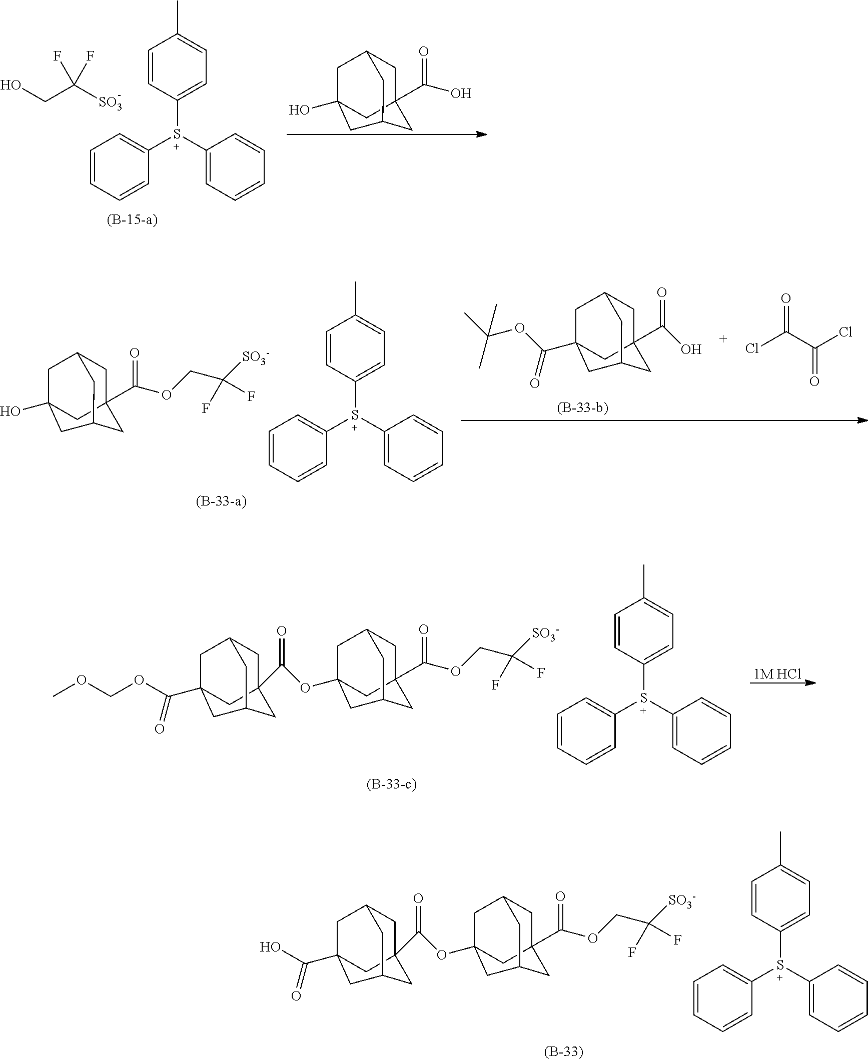

In a reaction vessel, 20.0 mmol of 3-hydroxy-1-adamantane carboxylic acid, 25.0 mmol of 1-(3-dimethylaminopropyl)-3-ethylcarbodiimide hydrochloride, 5.0 mmol of 4-dimethylaminopyridine, and 50 g of dichloromethane were added to 20.0 mmol of the onium salt compound (B-15-a) represented by the formula (B-15-a), and the mixture was reacted at room temperature for 12 hours. Thereafter, a 1 M aqueous hydrochloric acid solution was added to stop the reaction, followed by addition of dichloromethane for extraction, and then the organic layer was separated. After drying over sodium sulfate, the solvent was distilled off, and the residue was purified by column chromatography to obtain a compound (B-33-a) in a good yield.

20.0 mmol of the compound (B-33-b) represented by the formula (B-33-b), 20.0 mmol of oxalyl chloride, and 50 g of acetonitrile were added to a reaction vessel, and the mixture was stirred at 50° C. for 2 hours. Then, 30.0 mmol of the compound (B-33-a), 30.0 mmol of pyridine, and 3.0 mmol of 4-dimethylaminopyridine were added, and the mixture was stirred at 100° C. for 24 hours. Thereafter, water was added to stop the reaction, dichloromethane was then added thereto to perform extraction, and the organic layer was separated. The resulting organic layer was washed with a saturated aqueous solution of sodium chloride and then with water. After drying over sodium sulfate, the solvent was distilled off, and the residue was purified by column chromatography to obtain an onium salt compound (B-33-c) in a good yield.

To the onium salt compound (B-33-c) represented by the formula (B-33-c), 50 g of 1-M hydrochloric acid and 50 g of acetonitrile were added, and the mixture was stirred at 100° C. for 5 hours. Thereafter, water was added to dilute the mixture, followed by addition of dichloromethane and extraction, and then the organic layer was separated. The resulting organic layer was washed with a saturated aqueous solution of sodium chloride and then with water. After drying over sodium sulfate, the solvent was distilled off, and purification was performed by column chromatography to obtain an onium salt compound (B-33) represented by the formula (B-33) in a good yield.

### Synthesis Examples B14 to B16

### Synthesis of Onium Salt Compounds (B-34) to (B-36)

Onium salt compounds (B-34) to (B-36) represented by the following formulas (B-34) to (B-36) were synthesized in the same manner as in Synthesis Example B13 except that the raw materials and the precursors were appropriately changed.

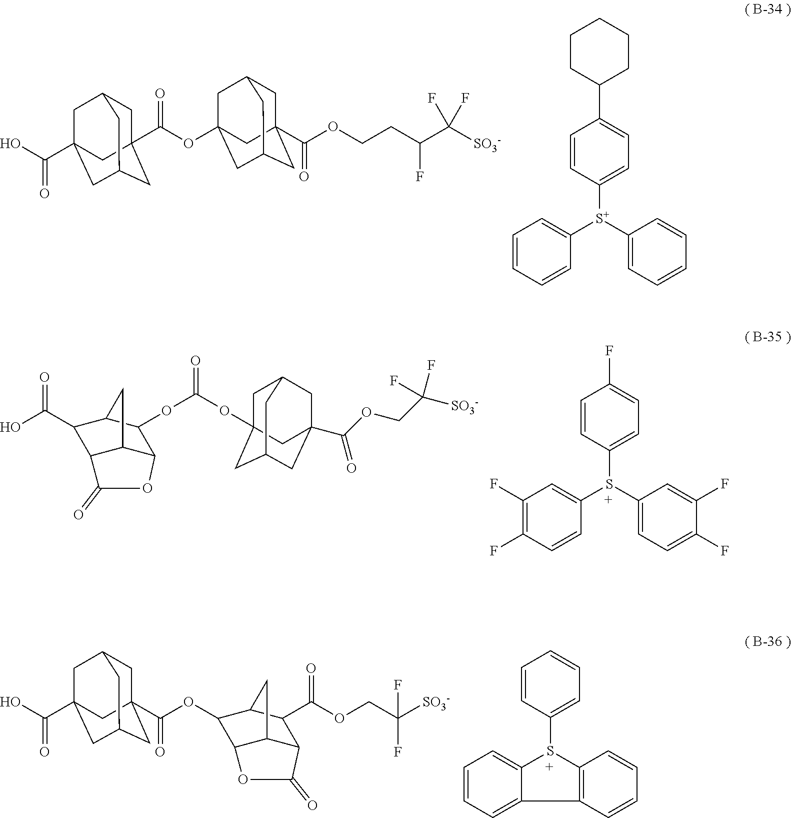

### Synthesis Example B17-a

### Synthesis of Compound (B-37-a)

A compound (B-37-a) was synthesized according to a synthesis scheme below.

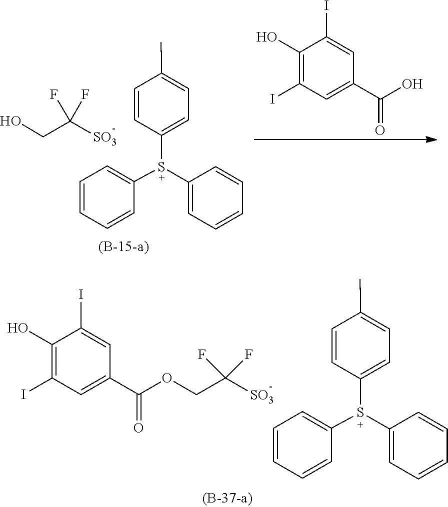

In a reaction vessel, 20.0 mmol of 4-hydroxy-3,5-diiodobenzene carboxylic acid, 25.0 mmol of 1-(3-dimethylaminopropyl)-3-ethylcarbodiimide hydrochloride, 5.0 mmol of 4-dimethylaminopyridine, and 50 g of dichloromethane were added to 20.0 mmol of the onium salt compound (B-15-a) represented by the formula (B-15-a), and the mixture was reacted at room temperature for 2 hours. Thereafter, a 1 M aqueous hydrochloric acid solution was added to stop the reaction, followed by addition of dichloromethane for extraction, and then the organic layer was separated. After drying over sodium sulfate, the solvent was distilled off, and the residue was purified by column chromatography to obtain a compound (B-37-a) in a good yield.

### Synthesis Example B17

### Synthesis of Compound (B-37)

A compound (B-37) was synthesized according to a synthesis scheme below.

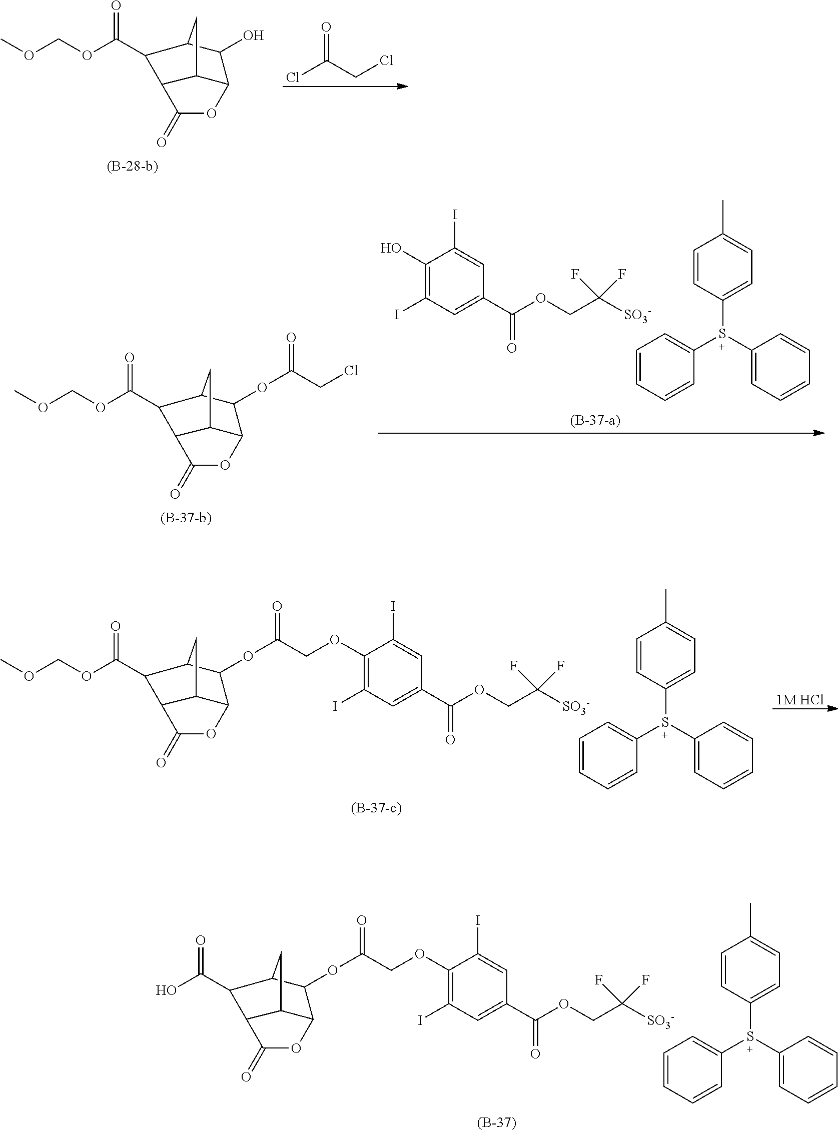

In a reaction vessel, 20.0 mmol of chloroacetyl chloride, 20.0 mmol of triethylamine, and 50 g of dichloromethane were added to 20.0 mmol of the compound (B-28-b) represented by the formula (B-28-b), and the mixture was reacted at room temperature for 1 hour. Thereafter, a 1 M aqueous hydrochloric acid solution was added to stop the reaction, followed by addition of dichloromethane for extraction, and then the organic layer was separated. After drying over sodium sulfate, the solvent was distilled off, and the residue was purified by column chromatography to obtain a compound (B-37-b) in a good yield.

To the compound (B-37-b) obtained above, 20.0 mmol of the compound (B-37-a) represented by the formula (B-37-a), 30.0 mmol of cesium carbonate, and 50 g of dimethylformamide were added, and the mixture was stirred at room temperature for 2 hours. Thereafter, a saturated aqueous solution of ammonium chloride was added to stop the reaction, followed by addition of dichloromethane for extraction, and then the organic layer was separated. The resulting organic layer was washed with a saturated aqueous solution of sodium chloride and then with water. After drying over sodium sulfate, the solvent was distilled off, and the residue was purified by column chromatography to obtain an onium salt compound (B-37-c) in a good yield.

To the onium salt compound (B-37-c) represented by the formula (B-37-c), 50 g of 1-M hydrochloric acid and 50 g of acetonitrile were added, and the mixture was stirred at 100° C. for 1 hour. Thereafter, water was added to dilute the mixture, followed by addition of dichloromethane and extraction, and then the organic layer was separated. The resulting organic layer was washed with a saturated aqueous solution of sodium chloride and then with water. After drying over sodium sulfate, the solvent was distilled off, and purification was performed by column chromatography to obtain an onium salt compound (B-37) represented by the formula (B-37) in a good yield.

The following compounds were used as components other than the synthesized components.

[Radiation-Sensitive Acid Generators Other than Radiation-Sensitive Acid Generators (B-1) to (B-37)]

b-1 to b-11: Compounds represented by the following formulas (b-1) to (b-11) (hereinafter, the compounds represented by the formulas (b-1) to (b-11) may be described as “compound (b-1)” to “compound (b-11)”, respectively).

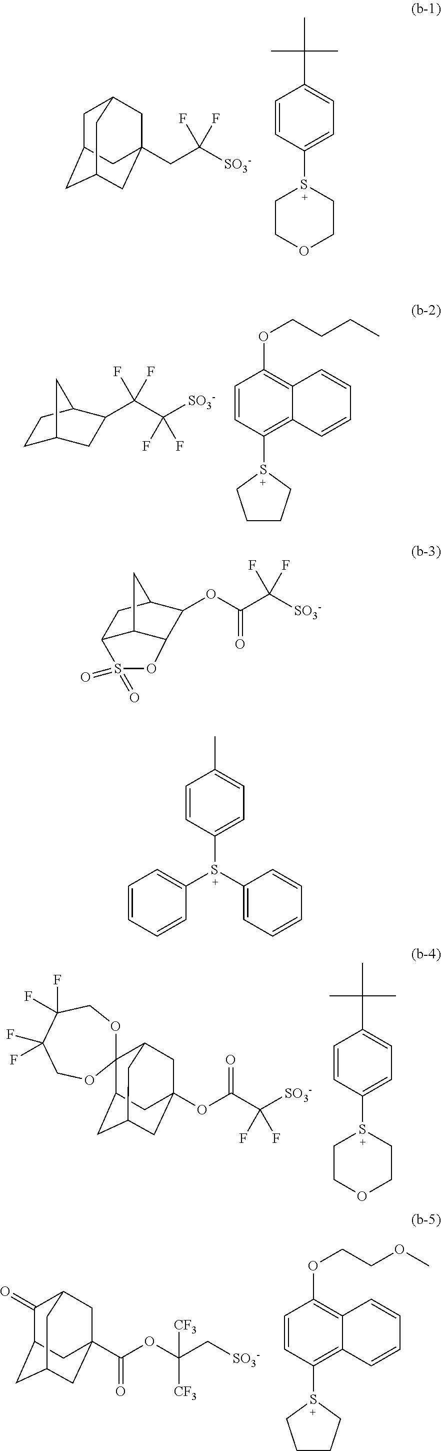

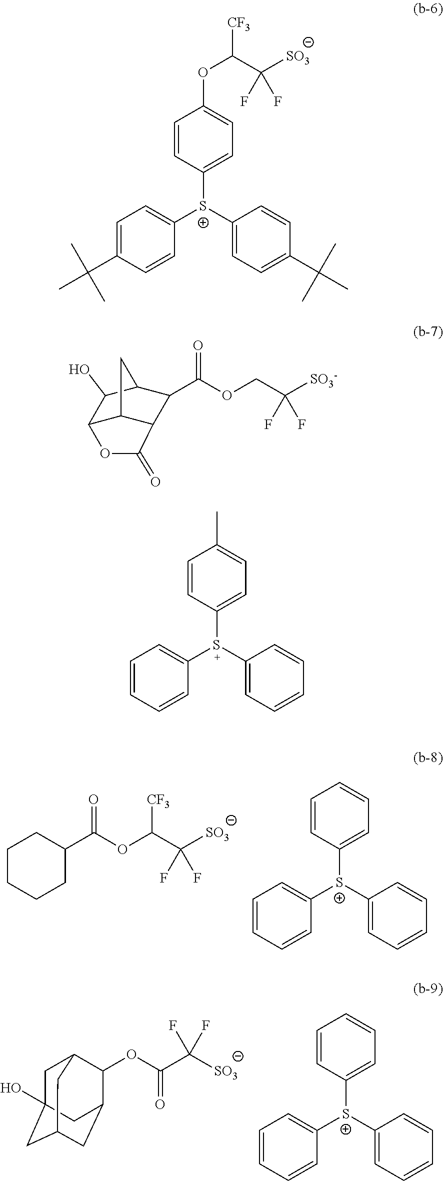

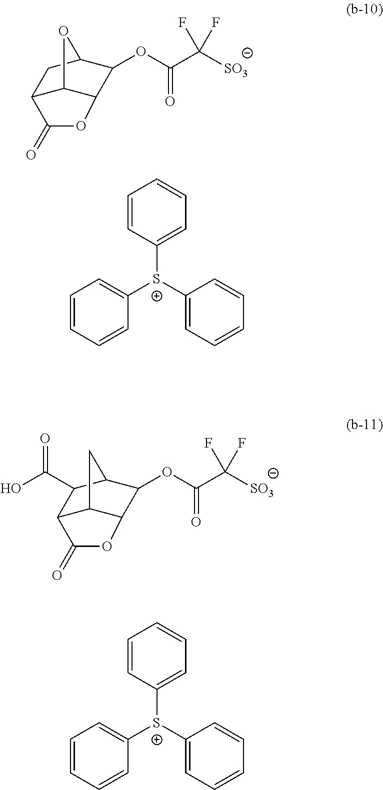

### [Acid Diffusion Controlling Agent (D)]

D-1 to D-12: Compounds represented by formulas (D-1) to (D-12) below

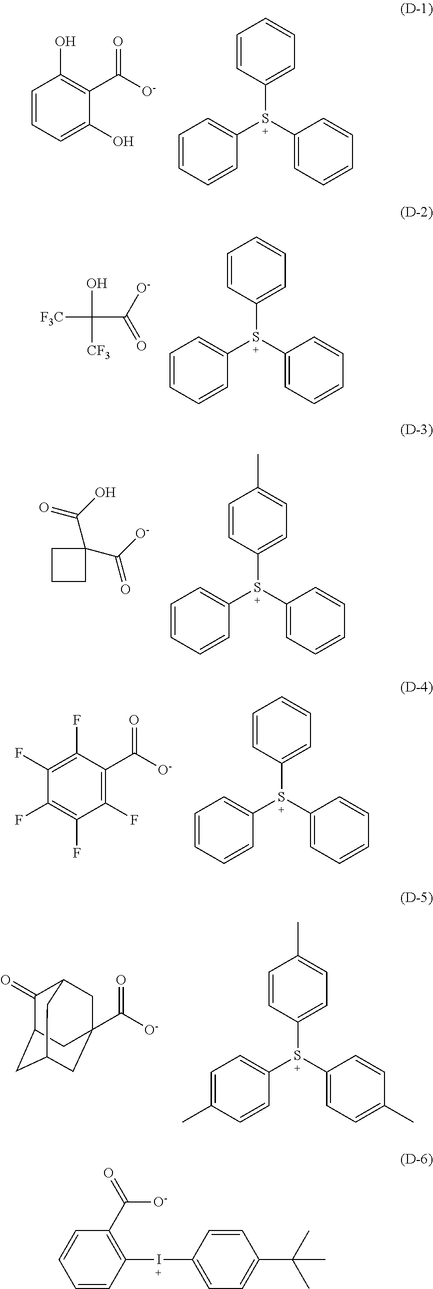

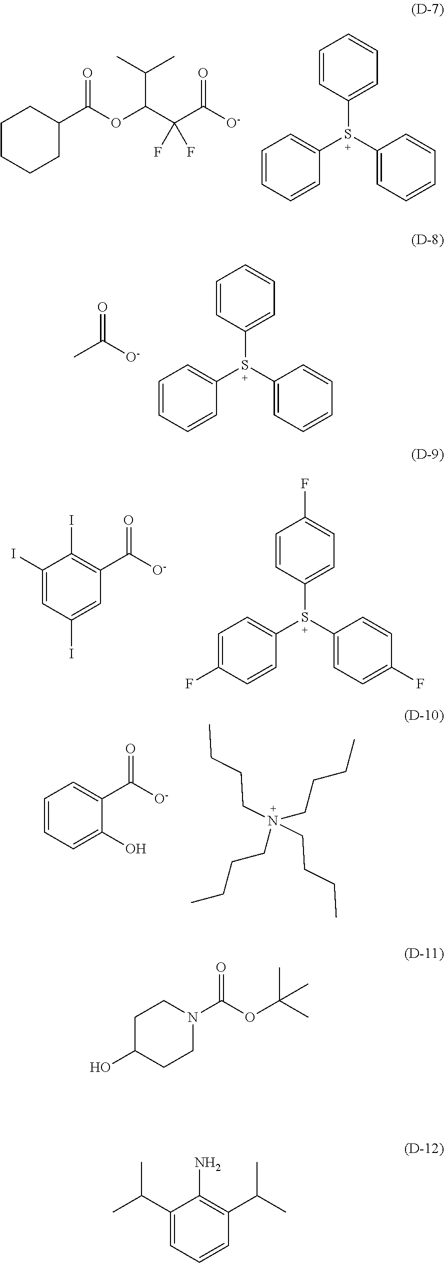

### [Solvent (E)]

E-1: Propylene glycol monomethyl ether acetate
E-2: Propylene glycol monomethyl ether
E-3: γ-Butyrolactone
E-4: Ethyl lactate
E-5: Diacetone alcohol
E-6: Methyl 2-hydroxyisobutyrate

### [Preparation of Positive Radiation-Sensitive Composition for ArF Immersion Exposure]

### Example 1

100 parts by mass of (A-1) as a polymer (A), 10.0 parts by mass of (B-1) as a radiation-sensitive acid generator (B), 4.0 parts by mass of (D-1) as an acid diffusion controlling agent (D), 5.0 parts by mass (solid content) of (F-1) as a high fluorine-content polymer (F), and 3,400 parts by mass of a mixed solvent of (E-1)/(E-2)/(E-3) as a solvent (E) were mixed, and the mixture was filtered through a membrane filter having a pore size of 0.2 μm to prepare a radiation-sensitive composition (J-1).

### Examples 2 to 53 and 101 to 114 and Comparative Examples 1 to 4 and 101 to 102

Radiation-sensitive compositions (J-2) to (J-53), (J-101) to (J-114), (CJ-1) to (CJ-4), and (CJ-101) to (CJ-102) were prepared in the same manner as in Example 1 except that the respective components of the types and contents shown in the following Table 4 were used.

TABLE 4

| | | | | | | Acid diffusion | | | | | |
|---|---|---|---|---|---|---|---|---|---|---|---|
| | | | | Radiation-sensitive | | controlling agent | | High fluorine- | | | |
| | | Polymer (A) | | acid generator (B) | | (D) | | content polymer (F) | | Solvent (E) | |
| | Radiation- | | Content | | Content | | Content | | Content | | |
| | sensitive | | (parts by | | (parts by | | (parts by | | (parts by | | Content |
| | composition | Type | mass) | Type | mass) | Type | mass) | Type | mass) | Type | (parts by mass) |
| Example 1 | J-1 | A-1 | 100 | B-1 | 10.0 | D-1 | 4.0 | F-1 | 5.0 | E-1/E-2/E-3 | 2240/960/200 |
| Example 2 | J-2 | A-2 | 100 | B-1 | 10.0 | D-1 | 4.0 | F-1 | 5.0 | E-1/E-2/E-3 | 2240/960/200 |
| Example 3 | J-3 | A-3 | 100 | B-1 | 10.0 | D-1 | 4.0 | F-1 | 5.0 | E-1/E-2/E-3 | 2240/960/200 |
| Example 4 | J-4 | A-4 | 100 | B-1 | 10.0 | D-1 | 4.0 | F-1 | 5.0 | E-1/E-2/E-3 | 2240/960/200 |
| Example 5 | J-5 | A-5 | 100 | B-1 | 10.0 | D-1 | 4.0 | F-1 | 5.0 | E-1/E-2/E-3 | 2240/960/200 |
| Example 6 | J-6 | A-6 | 100 | B-1 | 10.0 | D-1 | 4.0 | F-1 | 5.0 | E-1/E-2/E-3 | 2240/960/200 |
| Example 7 | J-7 | A-7 | 100 | B-1 | 10.0 | D-1 | 4.0 | F-1 | 5.0 | E-1/E-2/E-3 | 2240/960/200 |
| Example 8 | J-8 | A-8 | 100 | B-1 | 10.0 | D-1 | 4.0 | F-1 | 5.0 | E-1/E-2/E-3 | 2240/960/200 |
| Example 9 | J-9 | A-9 | 100 | B-1 | 10.0 | D-1 | 4.0 | F-1 | 5.0 | E-1/E-2/E-3 | 2240/960/200 |
| Example 10 | J-10 | A-10 | 100 | B-1 | 10.0 | D-1 | 4.0 | F-1 | 5.0 | E-1/E-2/E-3 | 2240/960/200 |
| Example 11 | J-11 | A-11 | 100 | B-1 | 10.0 | D-1 | 4.0 | F-1 | 5.0 | E-1/E-2/E-3 | 2240/960/200 |
| Example 12 | J-12 | A-1 | 100 | B-1 | 10.0 | D-2 | 4.0 | F-1 | 5.0 | E-1/E-2/E-3 | 2240/960/200 |
| Example 13 | J-13 | A-1 | 100 | B-1 | 10.0 | D-3 | 4.0 | F-1 | 5.0 | E-1/E-2/E-3 | 2240/960/200 |
| Example 14 | J-14 | A-1 | 100 | B-1 | 10.0 | D-4 | 4.0 | F-1 | 5.0 | E-1/E-2/E-3 | 2240/960/200 |
| Example 15 | J-15 | A-1 | 100 | B-1 | 10.0 | D-5 | 4.0 | F-1 | 5.0 | E-1/E-2/E-3 | 2240/960/200 |
| Example 16 | J-16 | A-1 | 100 | B-1 | 10.0 | D-6 | 4.0 | F-1 | 5.0 | E-1/E-2/E-3 | 2240/960/200 |
| Example 17 | J-17 | A-1 | 100 | B-1 | 10.0 | D-7 | 4.0 | F-1 | 5.0 | E-1/E-2/E-3 | 2240/960/200 |
| Example 101 | J-101 | A-1 | 100 | B-1 | 10.0 | D-9 | 4.0 | F-1 | 5.0 | E-1/E-2/E-3 | 2240/960/200 |
| Example 102 | J-101 | A-1 | 100 | B-1 | 10.0 | D-10 | 4.0 | F-1 | 5.0 | E-1/E-2/E-3 | 2240/960/200 |
| Example 18 | J-18 | A-1 | 100 | B-2 | 10.0 | D-1 | 4.0 | F-1 | 5.0 | E-1/E-2/E-3 | 2240/960/200 |
| Example 19 | J-19 | A-1 | 100 | B-3 | 10.0 | D-1 | 4.0 | F-1 | 5.0 | E-1/E-2/E-3 | 2240/960/200 |
| Example 20 | J-20 | A-1 | 100 | B-4 | 10.0 | D-1 | 4.0 | F-1 | 5.0 | E-1/E-2/E-3 | 2240/960/200 |
| Example 21 | J-21 | A-1 | 100 | B-5 | 10.0 | D-1 | 4.0 | F-1 | 5.0 | E-1/E-2/E-3 | 2240/960/200 |
| Example 22 | J-22 | A-1 | 100 | B-6 | 10.0 | D-1 | 4.0 | F-1 | 5.0 | E-1/E-2/E-3 | 2240/960/200 |
| Example 23 | J-23 | A-1 | 100 | B-7 | 10.0 | D-1 | 4.0 | F-1 | 5.0 | E-1/E-2/E-3 | 2240/960/200 |
| Example 24 | J-24 | A-1 | 100 | B-8 | 10.0 | D-1 | 4.0 | F-1 | 5.0 | E-1/E-2/E-3 | 2240/960/200 |
| Example 25 | J-25 | A-1 | 100 | B-9 | 10.0 | D-1 | 4.0 | F-1 | 5.0 | E-1/E-2/E-3 | 2240/960/200 |
| Example 26 | J-26 | A-1 | 100 | B-10 | 10.0 | D-1 | 4.0 | F-1 | 5.0 | E-1/E-2/E-3 | 2240/960/200 |
| Example 27 | J-27 | A-1 | 100 | B-11 | 10.0 | D-1 | 4.0 | F-1 | 5.0 | E-1/E-2/E-3 | 2240/960/200 |
| Example 28 | J-28 | A-1 | 100 | B-12 | 10.0 | D-1 | 4.0 | F-1 | 5.0 | E-1/E-2/E-3 | 2240/960/200 |
| Example 29 | J-29 | A-1 | 100 | B-13 | 10.0 | D-1 | 4.0 | F-1 | 5.0 | E-1/E-2/E-3 | 2240/960/200 |
| Example 30 | J-30 | A-1 | 100 | B-14 | 10.0 | D-1 | 4.0 | F-1 | 5.0 | E-1/E-2/E-3 | 2240/960/200 |
| Example 31 | J-31 | A-1 | 100 | B-15 | 10.0 | D-1 | 4.0 | F-1 | 5.0 | E-1/E-2/E-3 | 2240/960/200 |
| Example 32 | J-32 | A-1 | 100 | B-16 | 10.0 | D-1 | 4.0 | F-1 | 5.0 | E-1/E-2/E-3 | 2240/960/200 |
| Example 33 | J-33 | A-1 | 100 | B-17 | 10.0 | D-1 | 4.0 | F-1 | 5.0 | E-1/E-2/E-3 | 2240/960/200 |
| Example 34 | J-34 | A-1 | 100 | B-18 | 10.0 | D-1 | 4.0 | F-1 | 5.0 | E-1/E-2/E-3 | 2240/960/200 |
| Example 35 | J-35 | A-1 | 100 | B-19 | 10.0 | D-1 | 4.0 | F-1 | 5.0 | E-1/E-2/E-3 | 2240/960/200 |
| Example 36 | J-36 | A-1 | 100 | B-20 | 10.0 | D-1 | 4.0 | F-1 | 5.0 | E-1/E-2/E-3 | 2240/960/200 |
| Example 37 | J-37 | A-1 | 100 | B-21 | 10.0 | D-1 | 4.0 | F-1 | 5.0 | E-1/E-2/E-3 | 2240/960/200 |
| Example 38 | J-38 | A-1 | 100 | B-22 | 10.0 | D-1 | 4.0 | F-1 | 5.0 | E-1/E-2/E-3 | 2240/960/200 |
| Example 39 | J-39 | A-1 | 100 | B-23 | 10.0 | D-1 | 4.0 | F-1 | 5.0 | E-1/E-2/E-3 | 2240/960/200 |
| Example 40 | J-40 | A-1 | 100 | B-24 | 10.0 | D-1 | 4.0 | F-1 | 5.0 | E-1/E-2/E-3 | 2240/960/200 |
| Example 41 | J-41 | A-1 | 100 | B-25 | 10.0 | D-1 | 4.0 | F-1 | 5.0 | E-1/E-2/E-3 | 2240/960/200 |
| Example 42 | J-42 | A-1 | 100 | B-26 | 10.0 | D-1 | 4.0 | F-1 | 5.0 | E-1/E-2/E-3 | 2240/960/200 |
| Example 43 | J-43 | A-1 | 100 | B-27 | 10.0 | D-1 | 4.0 | F-1 | 5.0 | E-1/E-2/E-3 | 2240/960/200 |
| Example 103 | J-103 | A-1 | 100 | B-28 | 10.0 | D-1 | 4.0 | F-1 | 5.0 | E-1/E-2/E-3 | 2240/960/200 |
| Example 104 | J-104 | A-1 | 100 | B-29 | 10.0 | D-1 | 4.0 | F-1 | 5.0 | E-1/E-2/E-3 | 2240/960/200 |
| Example 105 | J-105 | A-1 | 100 | B-30 | 10.0 | D-1 | 4.0 | F-1 | 5.0 | E-1/E-2/E-3 | 2240/960/200 |
| Example 106 | J-106 | A-1 | 100 | B-31 | 10.0 | D-1 | 4.0 | F-1 | 5.0 | E-1/E-2/E-3 | 2240/960/200 |
| Example 107 | J-107 | A-1 | 100 | B-32 | 10.0 | D-1 | 4.0 | F-1 | 5.0 | E-1/E-2/E-3 | 2240/960/200 |
| Example 108 | J-108 | A-1 | 100 | B-33 | 10.0 | D-1 | 4.0 | F-1 | 5.0 | E-1/E-2/E-3 | 2240/960/200 |
| Example 109 | J-109 | A-1 | 100 | B-34 | 10.0 | D-1 | 4.0 | F-1 | 5.0 | E-1/E-2/E-3 | 2240/960/200 |
| Example 110 | J-110 | A-1 | 100 | B-35 | 10.0 | D-1 | 4.0 | F-1 | 5.0 | E-1/E-2/E-3 | 2240/960/200 |
| Example 111 | J-111 | A-1 | 100 | B-36 | 10.0 | D-1 | 4.0 | F-1 | 5.0 | E-1/E-2/E-3 | 2240/960/200 |
| Example 112 | J-112 | A-1 | 100 | B-37 | 10.0 | D-1 | 4.0 | F-1 | 5.0 | E-1/E-2/E-3 | 2240/960/200 |
| Example 44 | J-44 | A-1 | 100 | B-1/b-1 | 5.0/5.0 | D-1 | 4.0 | F-1 | 5.0 | E-1/E-2/E-3 | 2240/960/200 |
| Example 45 | J-45 | A-1 | 100 | B-1/b-2 | 5.0/5.0 | D-1 | 4.0 | F-1 | 5.0 | E-1/E-2/E-3 | 2240/960/200 |
| Example 46 | J-46 | A-1 | 100 | B-1/b-3 | 5.0/5.0 | D-1 | 4.0 | F-1 | 5.0 | E-1/E-2/E-3 | 2240/960/200 |
| Example 47 | J-47 | A-1 | 100 | B-1/b-4 | 5.0/5.0 | D-1 | 4.0 | F-1 | 5.0 | E-1/E-2/E-3 | 2240/960/200 |
| Example 48 | J-48 | A-1 | 100 | B-1/b-5 | 5.0/5.0 | D-1 | 4.0 | F-1 | 5.0 | E-1/E-2/E-3 | 2240/960/200 |
| Example 49 | J-49 | A-1 | 100 | B-1/b-6 | 5.0/5.0 | D-1 | 4.0 | F-1 | 5.0 | E-1/E-2/E-3 | 2240/960/200 |
| Example 50 | J-50 | A-1 | 100 | B-1/b-7 | 5.0/5.0 | D-1 | 4.0 | F-1 | 5.0 | E-1/E-2/E-3 | 2240/960/200 |
| Example 51 | J-51 | A-1 | 100 | B-1 | 10.0 | D-1 | 4.0 | F-2 | 5.0 | E-1/E-2/E-3 | 2240/960/200 |
| Example 52 | J-52 | A-1 | 100 | B-1 | 10.0 | D-1 | 4.0 | F-3 | 5.0 | E-1/E-2/E-3 | 2240/960/200 |
| Example 53 | J-53 | A-1 | 100 | B-1 | 10.0 | D-1 | 4.0 | F-4 | 5.0 | E-1/E-2/E-3 | 2240/960/200 |
| Example 113 | J-113 | A-1 | 100 | B-1 | 10.0 | D-1 | 4.0 | F-1 | 5.0 | E-1/E-4/E-3 | 2240/960/200 |
| Example 114 | J-114 | A-1 | 100 | B-1 | 10.0 | D-1 | 4.0 | F-1 | 5.0 | E-1/E-5/E-3 | 2240/960/200 |
| Comparative | CJ-1 | A-1 | 100 | b-8 | 10.0 | D-1 | 4.0 | F-1 | 5.0 | E-1/E-2/E-3 | 2240/960/200 |
| Example 1 | | | | | | | | | | | |
| Comparative | CJ-2 | A-1 | 100 | b-9 | 10.0 | D-1 | 4.0 | F-1 | 5.0 | E-1/E-2/E-3 | 2240/960/200 |
| Example 2 | | | | | | | | | | | |
| Comparative | CJ-3 | A-1 | 100 | b-10 | 10.0 | D-1 | 4.0 | F-1 | 5.0 | E-1/E-2/E-3 | 2240/960/200 |
| Example 3 | | | | | | | | | | | |
| Comparative | CJ-4 | A-1 | 100 | b-11 | 10.0 | D-8 | 4.0 | F-1 | 5.0 | E-1/E-2/E-3 | 2240/960/200 |
| Example 4 | | | | | | | | | | | |
| Comparative | CJ-101 | A-1 | 100 | b-11 | 10.0 | D-11 | 4.0 | F-1 | 5.0 | E-1/E-2/E-3 | 2240/960/200 |
| Example 101 | | | | | | | | | | | |
| Comparative | CJ-102 | A-1 | 100 | b-11 | 10.0 | D-12 | 4.0 | F-1 | 5.0 | E-1/E-2/E-3 | 2240/960/200 |
| Example 102 | | | | | | | | | | | |

### <Formation of Resist Pattern Using Positive Radiation-Sensitive Composition for ArF Immersion Exposure>

Onto the surface of a 12-inch silicon wafer, an underlayer antireflection film forming composition (“ARC66” manufactured by Brewer Science, Inc.) was applied with use of a spin coater (“CLEAN TRACK ACT12” manufactured by Tokyo Electron Ltd.). The wafer was then heated at 205° C. for 60 seconds to form an underlayer antireflection film having an average thickness of 100 nm. The positive radiation-sensitive composition for ArF immersion exposure prepared above was applied onto the underlayer antireflection film with use of the spin coater, followed by performing PB (pre-baking) at 100° C. for 60 seconds. Thereafter, cooling was performed at 23° C. for 30 seconds to form a resist film having an average thickness of 120 nm. Next, the resist film was exposed through a 60 nm line- and -space mask pattern with use of an ArF excimer laser immersion exposure apparatus (“TWINSCAN XT-1900i” manufactured by ASML) with NA of 1.35 under an optical condition of Dipole (σ=0.9/0.7). After the exposure, PEB (post exposure bake) was performed at 100° C. for 60 seconds. Thereafter, the resist film was developed with an alkali with use of a 2.38% by mass aqueous tetramethylammonium hydroxide (TMAH) solution as an alkaline developer, followed by washing with water and further drying to form a positive resist pattern (50 nm line- and -space pattern).

### Evaluation

The resist pattern formed using the positive radiation-sensitive composition for ArF immersion exposure was evaluated on sensitivity, LWR performance, pattern rectangularity, MEEF and Depth of focus in accordance with the following methods. The results are shown in the following Table 5. Note that a scanning electron microscope (“CG-5000” manufactured by Hitachi High-Tech Corporation) was used for measurement of the resist pattern. The evaluation results are shown in the following Table 5.

### [Sensitivity]

An exposure dose at which a 50 nm line- and -space pattern was formed in the resist pattern formation using the positive radiation-sensitive composition for ArF immersion exposure was defined as an optimum exposure dose, and this optimum exposure dose was defined as sensitivity (mJ/cm²). In the case of being 15 mJ/cm² or more and 30 mJ/cm² or less, the sensitivity was evaluated as “good”, and in the case of being less than 15 mJ/cm² or more than 30 mJ/cm², the sensitivity was evaluated as “poor”.

### [LWR Performance]

A 50 nm line- and -space resist pattern was formed by irradiation with the optimum exposure dose determined in the evaluation of the sensitivity. The formed resist pattern was observed from above the pattern using the scanning electron microscope. The variation in line width was measured at 500 points in total, the value of 30 was obtained from the distribution of the measured values, and the value of 30 was defined as LWR (nm). A smaller value of LWR indicates smaller roughness of the line and better performance. The LWR performance was evaluated as “good” when the LWR was 3.0 nm or less, and was evaluated as “poor” when the LWR exceeded 3.0 nm.

### [Pattern Rectangularity]

The 50 nm line- and -space resist pattern formed by irradiation with the optimum exposure dose determined in the evaluation of the sensitivity was observed using the scanning electron microscope, and the sectional shape of the line- and -space pattern was evaluated. The rectangularity of the resist pattern was evaluated as “A” (extremely good) when the ratio of the length of the lower side to the length of the upper side in the sectional shape was 1.00 or more and 1.05 or less, “B” (good) when the ratio was more than 1.05 and 1.10 or less, and “C” (poor) when the ratio was more than 1.10.

### [MEEF]

In the resist pattern to be resolved by irradiation in the optimum exposure dose, the slope of the straight line when the line width of the resist pattern formed using mask patterns having line widths of 52 nm, 54 nm, 56 nm, 58 nm, and 60 nm was plotted on the vertical axis, and the size of the mask patterns was plotted on the horizontal axis was calculated and used as MEEF. MEEF indicates better mask reproducibility as the value is closer to 1. In the case of being 2 or less, MEEF was evaluated as “good”, and in the case of being more than 2, the MEEF performance was evaluated as “poor”.

### [Depth of Focus]

In the resist pattern resolved at the optimum exposure dose determined in the evaluation of the sensitivity, a range of focal depth (DOF) at which the line width of the formed resist pattern is 45 nm or more and 55 nm or less was measured. A focal depth of 100 nm or more was evaluated as “A” (extremely good), a focal depth of 80 nm or more and less than 100 nm was evaluated as “B” (good), and a focal depth of less than 80 nm was evaluated as “C” (poor).

TABLE 5

| | Radiation- | | | | | |
|---|---|---|---|---|---|---|
| | sensitive | Sensitivity | LWR | Pattern | | |
| | composition | (mJ/cm²) | (nm) | rectangularity | MEEF | DOF |
| Example 1 | J-1 | 17 | 1.7 | A | 1.77 | A |
| Example 2 | J-2 | 16 | 1.7 | A | 1.67 | A |
| Example 3 | J-3 | 20 | 2.3 | A | 1.58 | A |
| Example 4 | J-4 | 21 | 2.4 | A | 1.72 | A |
| Example 5 | J-5 | 16 | 1.6 | A | 1.66 | A |
| Example 6 | J-6 | 18 | 1.6 | A | 1.66 | A |
| Example 7 | J-7 | 19 | 2.2 | A | 1.67 | A |
| Example 8 | J-8 | 20 | 1.5 | A | 1.77 | A |
| Example 9 | J-9 | 22 | 2.0 | A | 1.58 | A |
| Example 10 | J-10 | 21 | 2.0 | A | 1.77 | A |
| Example 11 | J-11 | 19 | 1.5 | A | 1.56 | A |
| Example 12 | J-12 | 22 | 1.7 | A | 1.72 | A |
| Example 13 | J-13 | 21 | 1.9 | A | 1.82 | A |
| Example 14 | J-14 | 24 | 1.5 | A | 1.77 | A |
| Example 15 | J-15 | 18 | 1.8 | A | 1.60 | A |
| Example 16 | J-16 | 21 | 2.2 | A | 1.67 | A |
| Example 17 | J-17 | 20 | 1.6 | A | 1.67 | A |
| Example 101 | J-101 | 25 | 2.2 | A | 1.77 | A |
| Example 102 | J-101 | 28 | 2.8 | A | 1.90 | A |
| Example 18 | J-18 | 18 | 2.0 | A | 1.61 | A |
| Example 19 | J-19 | 19 | 1.5 | A | 1.50 | A |
| Example 20 | J-20 | 23 | 2.0 | A | 1.69 | A |
| Example 21 | J-21 | 25 | 2.2 | A | 1.79 | A |
| Example 22 | J-22 | 22 | 2.1 | A | 1.70 | A |
| Example 23 | J-23 | 22 | 2.4 | A | 1.66 | A |
| Example 24 | J-24 | 25 | 2.1 | A | 1.69 | A |
| Example 25 | J-25 | 27 | 1.5 | A | 1.76 | A |
| Example 26 | J-26 | 25 | 2.2 | A | 1.74 | A |
| Example 27 | J-27 | 19 | 2.2 | A | 1.67 | A |
| Example 28 | J-28 | 20 | 2.3 | A | 1.75 | A |
| Example 29 | J-29 | 21 | 2.3 | A | 1.61 | A |
| Example 30 | J-30 | 26 | 1.5 | A | 1.50 | A |
| Example 31 | J-31 | 29 | 1.8 | A | 1.53 | A |
| Example 32 | J-32 | 28 | 2.1 | A | 1.69 | A |
| Example 33 | J-33 | 25 | 2.3 | A | 1.79 | A |
| Example 34 | J-34 | 27 | 2.3 | A | 1.77 | A |
| Example 35 | J-35 | 20 | 1.5 | A | 1.59 | A |
| Example 36 | J-36 | 27 | 1.9 | A | 1.62 | A |
| Example 37 | J-37 | 22 | 1.8 | A | 1.72 | A |
| Example 38 | J-38 | 26 | 2.4 | A | 1.62 | A |
| Example 39 | J-39 | 28 | 2.4 | A | 1.57 | A |
| Example 40 | J-40 | 25 | 2.0 | A | 1.51 | A |
| Example 41 | J-41 | 20 | 2.2 | A | 1.65 | A |
| Example 42 | J-42 | 19 | 2.8 | A | 1.70 | A |
| Example 43 | J-43 | 20 | 2.7 | A | 1.56 | A |
| Example 103 | J-103 | 18 | 2.1 | A | 1.35 | A |
| Example 104 | J-104 | 19 | 1.8 | A | 1.29 | A |
| Example 105 | J-105 | 17 | 1.9 | A | 1.37 | A |
| Example 106 | J-106 | 20 | 2.1 | A | 1.31 | A |
| Example 107 | J-107 | 21 | 2.3 | A | 1.28 | A |
| Example 108 | J-108 | 19 | 1.5 | A | 1.29 | A |
| Example 109 | J-109 | 22 | 1.9 | A | 1.49 | A |
| Example 110 | J-110 | 20 | 1.8 | A | 1.37 | A |
| Example 111 | J-111 | 19 | 1.9 | A | 1.21 | A |
| Example 112 | J-112 | 20 | 2.0 | A | 1.23 | A |
| Example 44 | J-44 | 25 | 2.1 | A | 1.70 | A |
| Example 45 | J-45 | 25 | 1.8 | A | 1.79 | A |
| Example 46 | J-46 | 24 | 1.9 | A | 1.71 | A |
| Example 47 | J-47 | 23 | 2.1 | A | 1.54 | A |
| Example 48 | J-48 | 25 | 2.4 | A | 1.73 | A |
| Example 49 | J-49 | 20 | 1.9 | A | 1.51 | A |
| Example 50 | J-50 | 19 | 1.9 | A | 1.65 | A |
| Example 51 | J-51 | 23 | 2.0 | A | 1.66 | A |
| Example 52 | J-52 | 25 | 2.1 | A | 1.78 | A |
| Example 53 | J-53 | 23 | 1.9 | A | 1.65 | A |
| Example 113 | J-113 | 24 | 1.7 | A | 1.76 | A |
| Example 114 | J-114 | 23 | 1.7 | A | 1.67 | A |
| Comparative | CJ-1 | 40 | 3.6 | B | 2.67 | C |
| Example 1 | | | | | | |
| Comparative | CJ-2 | 39 | 3.4 | C | 2.25 | C |
| Example 2 | | | | | | |
| Comparative | CJ-3 | 41 | 3.5 | C | 2.67 | B |
| Example 3 | | | | | | |
| Comparative | CJ-4 | 37 | 4.0 | C | 2.55 | C |
| Example 4 | | | | | | |
| Comparative | CJ-101 | 49 | 4.5 | C | 2.66 | C |
| Example 101 | | | | | | |
| Comparative | CJ-102 | 48 | 4.3 | C | 2.89 | C |
| Example 102 | | | | | | |

As is apparent from the results in Table 5, the radiation-sensitive compositions of Examples were good in sensitivity, LWR performance, pattern rectangularity, MEEF, and DOF when used for ArF immersion exposure, whereas the radiation-sensitive compositions of Comparative Examples were inferior in each characteristic to Examples. Therefore, when the radiation-sensitive compositions of Examples are used for ArF immersion exposure, a resist pattern having good LWR performance, pattern rectangularity, MEEF, and DOF with optimum sensitivity can be formed.

### [Preparation of Positive Radiation-Sensitive Composition for ArF-Dry Exposure]

### Example 54

100 parts by mass of (A-1) as a polymer (A), 6.0 parts by mass of (B-1) as a radiation-sensitive acid generator (B), 3.0 parts by mass of (D-2) as an acid diffusion controlling agent (D), and 3,230 parts by mass of a mixed solvent of (E-1)/(E-2)/(E-3) as a solvent (E) were mixed, and the mixture was filtered through a membrane filter having a pore size of 0.2 μm to prepare a radiation-sensitive composition (J-54).

### Examples 55 to 67 and Comparative Examples 5 to 8

Radiation-sensitive compositions (J-55) to (J-67) and (CJ-5) to (CJ-8) were prepared in the same manner as in Example 54 except that the components of the types and contents shown in the following Table 6 were used.

TABLE 6

| | | | | Radiation-sensitive | | Acid diffusion | | | |
|---|---|---|---|---|---|---|---|---|---|
| | | Polymer (A) | | acid generator (B) | | controlling agent (D) | | | |
| | Radiation- | | Content | | Content | | Content | Solvent (E) | |
| | sensitive | | (parts by | | (parts by | | (parts by | | Content |
| | composition | Type | mass) | Type | mass) | Type | mass) | Type | (parts by mass) |
| Example 54 | J-54 | A-1 | 100 | B-1 | 6.0 | D-2 | 3.0 | E-1/E-2/E-3 | 2240/960/30 |
| Example 55 | J-55 | A-6 | 100 | B-1 | 6.0 | D-2 | 3.0 | E-1/E-2/E-3 | 2240/960/30 |
| Example 56 | J-56 | A-7 | 100 | B-1 | 6.0 | D-2 | 3.0 | E-1/E-2/E-3 | 2240/960/30 |
| Example 57 | J-57 | A-8 | 100 | B-1 | 6.0 | D-2 | 3.0 | E-1/E-2/E-3 | 2240/960/30 |
| Example 58 | J-58 | A-1 | 100 | B-1 | 6.0 | D-6 | 3.0 | E-1/E-2/E-3 | 2240/960/30 |
| Example 59 | J-59 | A-1 | 100 | B-1 | 6.0 | D-7 | 3.0 | E-1/E-2/E-3 | 2240/960/30 |
| Example 60 | J-60 | A-1 | 100 | B-7 | 6.0 | D-2 | 3.0 | E-1/E-2/E-3 | 2240/960/30 |
| Example 61 | J-61 | A-1 | 100 | B-14 | 6.0 | D-2 | 3.0 | E-1/E-2/E-3 | 2240/960/30 |
| Example 62 | J-62 | A-1 | 100 | B-21 | 6.0 | D-2 | 3.0 | E-1/E-2/E-3 | 2240/960/30 |
| Example 63 | J-63 | A-1 | 100 | B-25 | 6.0 | D-2 | 3.0 | E-1/E-2/E-3 | 2240/960/30 |
| Example 64 | J-64 | A-1 | 100 | B-27 | 6.0 | D-2 | 3.0 | E-1/E-2/E-3 | 2240/960/30 |
| Example 65 | J-65 | A-1 | 100 | B-1/b-2 | 3.0/3.0 | D-2 | 3.0 | E-1/E-2/E-3 | 2240/960/30 |
| Example 66 | J-66 | A-1 | 100 | B-1/b-4 | 3.0/3.0 | D-2 | 3.0 | E-1/E-2/E-3 | 2240/960/30 |
| Example 67 | J-67 | A-1 | 100 | B-1/B-10 | 3.0/3.0 | D-2 | 3.0 | E-1/E-2/E-3 | 2240/960/30 |
| Comparative | CJ-5 | A-1 | 100 | b-8 | 6.0 | D-2 | 3.0 | E-1/E-2/E-3 | 2240/960/30 |
| Example 5 | | | | | | | | | |
| Comparative | CJ-6 | A-1 | 100 | b-9 | 6.0 | D-2 | 3.0 | E-1/E-2/E-3 | 2240/960/30 |
| Example 6 | | | | | | | | | |
| Comparative | CJ-7 | A-1 | 100 | b-10 | 6.0 | D-2 | 3.0 | E-1/E-2/E-3 | 2240/960/30 |
| Example 7 | | | | | | | | | |
| Comparative | CJ-8 | A-1 | 100 | b-11 | 6.0 | D-8 | 3.0 | E-1/E-2/E-3 | 2240/960/30 |
| Example 8 | | | | | | | | | |

### <Formation of Resist Pattern Using Positive Radiation-Sensitive Composition for ArF-Dry Exposure>

Onto the surface of an 8-inch silicon wafer, an underlayer antireflection film forming composition (“ARC29” manufactured by Brewer Science, Inc.) was applied with use of a spin coater (“CLEAN TRACK ACT8” manufactured by Tokyo Electron Ltd.). The wafer was then heated at 205° C. for 60 seconds to form an underlayer antireflection film having an average thickness of 77 nm. The positive radiation-sensitive composition for ArF-Dry exposure prepared above was applied onto the underlayer antireflection film with use of the spin coater, followed by performing PB (pre-baking) at 100° C. for 60 seconds. Thereafter, cooling was performed at 23° C. for 30 seconds to form a resist film having an average thickness of 200 nm. Next, a line- and -space resist pattern having a line width of 90 nm was formed on this resist film using an ArF excimer laser exposure apparatus (“S306C” manufactured by Nikon Corporation) at NA=0.75 under an optical condition of Annular (σ=0.8/0.6). After the exposure, PEB (post exposure bake) was performed at 100° C. for 60 seconds. Thereafter, the resist film was developed with an alkali with use of a 2.38% by mass aqueous TMAH solution as an alkaline developer, followed by washing with water and further drying to form a positive resist pattern (100 nm line- and -space resist pattern).

### Evaluation

The resist pattern formed using the positive radiation-sensitive composition for ArF-Dry exposure was evaluated on sensitivity, LWR performance, and pattern rectangularity in accordance with the following methods. The results are shown in the following Table 7. A scanning electron microscope (“S-9380” manufactured by Hitachi High-Tech Corporation) was used for measuring the length of the resist pattern.

### [Sensitivity]

An exposure dose at which a 100 nm line- and -space pattern was formed in the aforementioned resist pattern formation using each of the positive radiation-sensitive compositions for ArF-Dry exposure was defined as an optimum exposure dose, and this optimum exposure dose was defined as sensitivity (mJ/cm²). In the case of being 15 mJ/cm² or more and 30 mJ/cm² or less, the sensitivity was evaluated as “good”, and in the case of being less than 15 mJ/cm² or more than 30 mJ/cm², the sensitivity was evaluated as “poor”.

### [LWR Performance]

A 100 nm line- and -space resist pattern was formed by irradiation with the optimum exposure dose determined in the evaluation of the sensitivity. The formed resist pattern was observed from above the pattern using the scanning electron microscope. The variation in line width was measured at 500 points in total, the value of 30 was obtained from the distribution of the measured values, and the value of 30 was defined as LWR (nm). A smaller value of LWR indicates smaller roughness of the line and better performance. The LWR performance was evaluated as “good” when the LWR was 4.0 nm or less, and was evaluated as “poor” when the LWR exceeded 4.0 nm.

### [Pattern Rectangularity]

The 100 nm line- and -space resist pattern formed by irradiation with the optimum exposure dose determined in the evaluation of the sensitivity was observed using the scanning electron microscope, and the sectional shape of the line- and -space pattern was evaluated. The rectangularity of the resist pattern was evaluated as “A” (extremely good) when the ratio of the length of the lower side to the length of the upper side in the sectional shape was 1.00 or more and 1.05 or less, “B” (good) when the ratio was more than 1.05 and 1.10 or less, and “C” (poor) when the ratio was more than 1.10.

TABLE 7

| | Radiation-sensitive | Sensitivity | LWR | Pattern |
|---|---|---|---|---|
| | composition | (mJ/cm²) | (nm) | rectangularity |
| Example 54 | J-54 | 15 | 3.3 | A |
| Example 55 | J-55 | 15 | 3.6 | A |
| Example 56 | J-56 | 27 | 3.2 | A |
| Example 57 | J-57 | 17 | 3.4 | A |
| Example 58 | J-58 | 24 | 3.6 | A |
| Example 59 | J-59 | 16 | 3.3 | A |
| Example 60 | J-60 | 24 | 3.3 | A |
| Example 61 | J-61 | 17 | 2.8 | A |
| Example 62 | J-62 | 21 | 3.5 | A |
| Example 63 | J-63 | 21 | 3.0 | A |
| Example 64 | J-64 | 18 | 3.5 | A |
| Example 65 | J-65 | 17 | 2.7 | A |
| Example 66 | J-66 | 16 | 3.0 | A |
| Example 67 | J-67 | 20 | 3.6 | A |
| Comparative | CJ-5 | 35 | 4.6 | C |
| Example 5 | | | | |
| Comparative | CJ-6 | 37 | 4.7 | C |
| Example 6 | | | | |
| Comparative | CJ-7 | 35 | 4.5 | C |
| Example 7 | | | | |
| Comparative | CJ-8 | 36 | 4.6 | C |
| Example 8 | | | | |

As is apparent from the results in Table 7, the radiation-sensitive compositions of Examples were good in sensitivity, LWR performance, and pattern rectangularity when used for ArF-Dry exposure, whereas the radiation-sensitive compositions of Comparative Examples were inferior in each characteristic to Examples. Therefore, when the radiation-sensitive compositions of Examples are used for ArF-Dry exposure, a resist pattern having good LWR performance and pattern rectangularity with optimum sensitivity can be formed.

### [Preparation of Positive Radiation-Sensitive Composition for Extreme Ultraviolet (EUV) Exposure]

### Example 68

100 parts by mass of (A-12) as a polymer (A), 20.0 parts by mass of (B-1) as a radiation-sensitive acid generator (B), 10.0 parts by mass of (D-4) as an acid diffusion controlling agent (D), 3.0 parts by mass (solid content) of (F-5) as a high fluorine-content polymer (F), and 6, 110 parts by mass of a mixed solvent of (E-1)/(E-4) as a solvent (E) were mixed, and the mixture was filtered through a membrane filter having a pore size of 0.2 μm to prepare a radiation-sensitive composition (J-68).

### Examples 69 to 78 and 115 to 127 and Comparative Examples 9 to 12

Radiation-sensitive compositions (J-69) to (J-78), (J-115) to (J-127), and (CJ-9) to (CJ-12) were prepared in the same manner as in Example 68 except that the respective components of the types and contents shown in the following Table 8 were used.

TABLE 8

| | | | | | | Acid diffusion | | High fluorine- | | | |
|---|---|---|---|---|---|---|---|---|---|---|---|
| | | Polymer (A) | | Radiation-sensitive | | controlling agent | | content polymer | | | |
| | | | Content | acid generator (B) | | (D) | | (F) | | | |
| | Radiation- | | (parts | | Content | | Content | | Content | Solvent (E) | |
| | sensitive | | by | | (parts | | (parts by | | (parts by | | Content |
| | composition | Type | mass) | Type | by mass) | Type | mass) | Type | mass) | Type | (parts by mass) |
| Example 68 | J-68 | A-12 | 100 | B-1 | 20.0 | D-4 | 10.0 | F-5 | 3.0 | E-1/E-4 | 4280/1830 |
| Example 69 | J-69 | A-12 | 100 | B-6 | 20.0 | D-4 | 10.0 | F-5 | 3.0 | E-1/E-4 | 4280/1830 |
| Example 70 | J-70 | A-12 | 100 | B-9 | 20.0 | D-4 | 10.0 | F-5 | 3.0 | E-1/E-4 | 4280/1830 |
| Example 71 | J-71 | A-12 | 100 | B-10 | 20.0 | D-4 | 10.0 | F-5 | 3.0 | E-1/E-4 | 4280/1830 |
| Example 72 | J-72 | A-12 | 100 | B-22 | 20.0 | D-4 | 10.0 | F-5 | 3.0 | E-1/E-4 | 4280/1830 |
| Example 73 | J-73 | A-12 | 100 | B-23 | 20.0 | D-4 | 10.0 | F-5 | 3.0 | E-1/E-4 | 4280/1830 |
| Example 115 | J-115 | A-12 | 100 | B-28 | 20.0 | D-4 | 10.0 | F-5 | 3.0 | E-1/E-4 | 4280/1830 |
| Example 116 | J-116 | A-12 | 100 | B-30 | 20.0 | D-4 | 10.0 | F-5 | 3.0 | E-1/E-4 | 4280/1830 |
| Example 117 | J-117 | A-12 | 100 | B-31 | 20.0 | D-4 | 10.0 | F-5 | 3.0 | E-1/E-4 | 4280/1830 |
| Example 118 | J-118 | A-12 | 100 | B-32 | 20.0 | D-4 | 10.0 | F-5 | 3.0 | E-1/E-4 | 4280/1830 |
| Example 119 | J-119 | A-12 | 100 | B-34 | 20.0 | D-4 | 10.0 | F-5 | 3.0 | E-1/E-4 | 4280/1830 |
| Example 120 | J-120 | A-12 | 100 | B-36 | 20.0 | D-4 | 10.0 | F-5 | 3.0 | E-1/E-4 | 4280/1830 |
| Example 121 | J-121 | A-12 | 100 | B-37 | 20.0 | D-4 | 10.0 | F-5 | 3.0 | E-1/E-4 | 4280/1830 |
| Example 122 | J-122 | A-12 | 100 | B-30 | 50.0 | D-4 | 35.0 | F-5 | 3.0 | E-1/E-4 | 4280/1830 |
| Example 123 | J-123 | A-12 | 100 | B-30 | 20.0 | D-1 | 10.0 | F-5 | 3.0 | E-1/E-4 | 4280/1830 |
| Example 124 | J-124 | A-12 | 100 | B-30 | 20.0 | D-6 | 10.0 | F-5 | 3.0 | E-1/E-4 | 4280/1830 |
| Example 125 | J-125 | A-12 | 100 | B-30 | 20.0 | D-9 | 10.0 | F-5 | 3.0 | E-1/E-4 | 4280/1830 |
| Example 126 | J-126 | A-12 | 100 | B-30 | 20.0 | D-4 | 10.0 | F-5 | 3.0 | E-1/E-2/E-5 | 2000/4000/30 |
| Example 127 | J-127 | A-12 | 100 | B-30 | 20.0 | D-4 | 10.0 | F-5 | 3.0 | E-1/E-2/E-6 | 2000/4000/30 |
| Example 74 | J-74 | A-12 | 100 | B-19/b-5 | 10.0/10.0 | D-4 | 10.0 | F-5 | 3.0 | E-1/E-4 | 4280/1830 |
| Example 75 | J-75 | A-12 | 100 | B-15/B-25 | 10.0/10.0 | D-4 | 10.0 | F-5 | 3.0 | E-1/E-4 | 4280/1830 |
| Example 76 | J-76 | A-13 | 100 | B-1 | 20.0 | D-4 | 10.0 | F-5 | 3.0 | E-1/E-4 | 4280/1830 |
| Example 77 | J-77 | A-14 | 100 | B-1 | 20.0 | D-4 | 10.0 | F-5 | 3.0 | E-1/E-4 | 4280/1830 |
| Example 78 | J-78 | A-15 | 100 | B-1 | 20.0 | D-4 | 10.0 | F-5 | 3.0 | E-1/E-4 | 4280/1830 |
| Comparative | CJ-9 | A-12 | 100 | b-8 | 20.0 | D-4 | 10.0 | F-5 | 3.0 | E-1/E-4 | 4280/1830 |
| Example 9 | | | | | | | | | | | |
| Comparative | CJ-10 | A-12 | 100 | b-9 | 20.0 | D-4 | 10.0 | F-5 | 3.0 | E-1/E-4 | 4280/1830 |
| Example 10 | | | | | | | | | | | |
| Comparative | CJ-11 | A-12 | 100 | b-10 | 20.0 | D-4 | 10.0 | F-5 | 3.0 | E-1/E-4 | 4280/1830 |
| Example 11 | | | | | | | | | | | |
| Comparative | CJ-12 | A-12 | 100 | b-11 | 20.0 | D-8 | 10.0 | F-5 | 3.0 | E-1/E-4 | 4280/1830 |
| Example 12 | | | | | | | | | | | |

### <Formation of Resist Pattern Using Positive Radiation-Sensitive Composition for EUV Exposure>

Onto the surface of a 12-inch silicon wafer, an underlayer antireflection film forming composition (“ARC66” manufactured by Brewer Science, Inc.) was applied with use of a spin coater (“CLEAN TRACK ACT12” manufactured by Tokyo Electron Ltd.). The wafer was then heated at 205° C. for 60 seconds to form an underlayer antireflection film having an average thickness of 105 nm. The positive radiation-sensitive composition for EUV exposure prepared above was applied onto the underlayer antireflection film with use of the spin coater, followed by performing PB at 130° C. for 60 seconds. Thereafter, cooling was performed at 23° C. for 30 seconds to form a resist film having an average thickness of 50 nm. Next, the resist film was exposed by an EUV exposure apparatus (“NXE3300”, manufactured by ASML) with NA of 0.33 under a lighting condition of Conventional s=0.89 and with a mask of imecDEFECT32FFR02. After the exposure, PEB was performed at 120° C. for 60 seconds. Thereafter, the resist film was developed with an alkali with use of a 2.38% by mass aqueous TMAH solution as an alkaline developer, followed by washing with water and further drying to form a positive resist pattern (15 nm line- and -space pattern).

### Evaluation

The resist pattern formed using the positive radiation-sensitive composition for EUV exposure was evaluated on sensitivity, LWR performance, and pattern rectangularity in accordance with the following methods. The results are shown in the following Table 9. Note that a scanning electron microscope (“CG-5000” manufactured by Hitachi High-Tech Corporation) was used for measurement of the resist pattern.

### [Sensitivity]

In formation of the resist pattern using the positive radiation-sensitive composition for EUV exposure, an exposure dose at which a 15 nm line- and -space pattern was formed was defined as an optimum exposure dose, and this optimum exposure dose was defined as sensitivity (mJ/cm²). In the case of being 25 mJ/cm² or more and 40 mJ/cm² or less, the sensitivity was evaluated as “good”, and in the case of being less than 25 mJ/cm² or more than 40 mJ/cm², the sensitivity was evaluated as “poor”.

### [LWR Performance]

A resist pattern was formed by adjusting a mask size so as to form a 15 nm line- and -space pattern by irradiation with the optimum exposure dose obtained in the evaluation of the sensitivity. The formed resist pattern was observed from above the pattern using the scanning electron microscope. The variation in line width was measured at 500 points in total, the value of 30 was obtained from the distribution of the measured values, and the value of 30 was defined as LWR (nm). The smaller the value of the LWR is, the smaller the wobble of the line is and the better the LWR is. The LWR performance was evaluated as “good” when the LWR was 4.0 nm or less, and was evaluated as “poor” when the LWR exceeded 4.0 nm.

### [Pattern Rectangularity]

The 15 nm line- and -space resist pattern formed by irradiation with the optimum exposure dose determined in the evaluation of the sensitivity was observed using the scanning electron microscope, and the sectional shape of the line- and -space pattern was evaluated. The rectangularity of the resist pattern was evaluated as “A” (extremely good) when the ratio of the length of the lower side to the length of the upper side in the sectional shape was 1.00 or more and 1.05 or less, “B” (good) when the ratio was more than 1.05 and 1.10 or less, and “C” (poor) when the ratio was more than 1.10.

TABLE 9

| | Radiation-sensitive | Sensitivity | LWR | Pattern |
|---|---|---|---|---|
| | composition | (mJ/cm²) | (nm) | rectangularity |
| Example 68 | J-68 | 35 | 2.7 | A |
| Example 69 | J-69 | 35 | 3.5 | A |
| Example 70 | J-70 | 36 | 3.1 | A |
| Example 71 | J-71 | 34 | 3.5 | A |
| Example 72 | J-72 | 32 | 3.6 | A |
| Example 73 | J-73 | 34 | 3.0 | A |
| Example 115 | J-115 | 27 | 2.4 | A |
| Example 116 | J-116 | 28 | 2.5 | A |
| Example 117 | J-117 | 29 | 2.4 | A |
| Example 118 | J-118 | 27 | 2.6 | A |
| Example 119 | J-119 | 28 | 2.4 | A |
| Example 120 | J-120 | 29 | 2.5 | A |
| Example 121 | J-121 | 27 | 2.3 | A |
| Example 122 | J-122 | 28 | 2.3 | A |
| Example 123 | J-123 | 27 | 2.5 | A |
| Example 124 | J-124 | 28 | 2.3 | A |
| Example 125 | J-125 | 27 | 2.4 | A |
| Example 126 | J-126 | 29 | 2.3 | A |
| Example 127 | J-127 | 27 | 2.5 | A |
| Example 74 | J-74 | 34 | 3.2 | A |
| Example 75 | J-75 | 34 | 3.1 | A |
| Example 76 | J-76 | 35 | 2.7 | A |
| Example 77 | J-77 | 31 | 3.0 | A |
| Example 78 | J-78 | 30 | 3.3 | A |
| Comparative | CJ-9 | 45 | 4.5 | C |
| Example 9 | | | | |
| Comparative | CJ-10 | 44 | 4.7 | C |
| Example 10 | | | | |
| Comparative | CJ-11 | 46 | 4.3 | C |
| Example 11 | | | | |
| Comparative | CJ-12 | 43 | 4.6 | C |
| Example 12 | | | | |

As is apparent from the results in Table 9, the radiation-sensitive compositions of Examples were good in sensitivity, LWR performance, and pattern rectangularity when used for EUV exposure, whereas the radiation-sensitive compositions of Comparative Examples were inferior in the characteristics to those of Examples.

[Preparation of Negative Radiation-Sensitive Composition for ArF Exposure, and Formation and Evaluation of Resist Pattern Using this Composition]

### Example 79

100 parts by mass of (A-1) as a polymer (A), 12.0 parts by mass of (B-28) as a radiation-sensitive acid generator (B), 10.0 parts by mass of (D-7) as an acid diffusion controlling agent (D), 2.0 parts by mass (solid content) of (F-3) as a high fluorine-content polymer (F), and 3,230 parts by mass of a mixed solvent of (E-1)/(E-2)/(E-3) as a solvent (E) were mixed, and the mixture was filtered through a membrane filter having a pore size of 0.2 μm to prepare a radiation-sensitive composition (J-79).

Onto the surface of a 12-inch silicon wafer, an underlayer antireflection film forming composition (“ARC66” manufactured by Brewer Science, Inc.) was applied with use of a spin coater (“CLEAN TRACK ACT12” manufactured by Tokyo Electron Ltd.). The wafer was then heated at 205° C. for 60 seconds to form an underlayer antireflection film having an average thickness of 100 nm. The negative radiation-sensitive composition for ArF exposure (J-85) prepared above was applied onto the underlayer antireflection film with use of the spin coater, followed by performing PB (pre-baking) at 100° C. for 60 seconds. Thereafter, cooling was performed at 23° C. for 30 seconds to form a resist film having an average thickness of 120 nm. Next, this resist film was exposed through a mask pattern having a hole of 50 nm and a pitch of 100 nm using an ArF excimer laser immersion exposure apparatus (“TWINSCAN XT-1900i” manufactured by ASML) with NA of 1.35 under an optical condition of Annular (σ=0.8/0.6). After the exposure, PEB (post exposure bake) was performed at 100° C. for 60 seconds. Thereafter, the resist film was developed with an organic solvent using n-butyl acetate as an organic solvent developer, and dried to form a negative resist pattern (pattern of contact holes with a 55 nm hole and a 110 nm pitch).

The resist pattern using the negative radiation-sensitive composition for ArF exposure was evaluated on sensitivity in the same manner as in the evaluation of the resist pattern using the positive radiation-sensitive composition for ArF immersion exposure. In addition, CDU performance and pattern circularity were evaluated in accordance with the following methods.

### [CDU Performance]

Contact holes with a 55 nm hole and a 110 nm pitch were formed by irradiation with the optimum exposure dose determined in the evaluation of sensitivity. The formed resist pattern was observed from above the pattern using the scanning electron microscope. The variation in diameter of the contact holes was measured at 500 points in total. The 3 sigma value was determined from the distribution of the measurement values, and defined as CDU (nm). The smaller the value of the CDU is, the smaller the roughness of the contact holes is, which is better. When the value was less than 3.5 nm, the CDU performance was evaluated to be “good”, and when the value was 3.5 nm or more, the CDU performance was evaluated to be “poor”.

### [Pattern Circularity]

The contact holes with a 55 nm hole and a 110 nm pitch formed by irradiation with the optimum exposure dose determined in the evaluation of the sensitivity were observed in planar view with use of the scanning electron microscope, and the size in the longitudinal direction and the size in the lateral direction were measured. The pattern circularity was evaluated as “A” (extremely good) when the ratio of the size in the longitudinal direction to the size in the lateral direction was 0.95 or more and less than 1.05, “B” (good) when the ratio was 0.90 or more and less than 0.95, or 1.05 or more and less than 1.10, and “C” (poor) when the ratio was less than 0.90 or more than 1.10.

As a result, the radiation-sensitive composition of Example 79 had good sensitivity, CDU performance, and pattern circularity even when a negative resist pattern was formed by ArF exposure.

[Preparation of Negative Radiation-Sensitive Composition for EUV Exposure, Formation of Resist Pattern Using this Composition, and Evaluation]

### Example 80

100 parts by mass of (A-15) as a polymer (A), 55.0 parts by mass of (B-37) as a radiation-sensitive acid generator (B), 50.0 parts by mass of (D-6) as an acid diffusion controlling agent (D), 5.0 parts by mass (solid content) of (F-5) as a high fluorine-content polymer (F), and 6, 110 parts by mass of a mixed solvent of (E-1)/(E-4) as a solvent (E) were mixed, and the mixture was filtered through a membrane filter having a pore size of 0.2 μm to prepare a radiation-sensitive composition (J-80).

Onto the surface of a 12-inch silicon wafer, an underlayer antireflection film forming composition (“ARC66” manufactured by Brewer Science, Inc.) was applied with use of a spin coater (“CLEAN TRACK ACT12” manufactured by Tokyo Electron Ltd.). The wafer was then heated at 205° C. for 60 seconds to form an underlayer antireflection film having an average thickness of 105 nm. The negative radiation-sensitive composition for EUV exposure (J-80) prepared above was applied onto the underlayer antireflection film with use of the spin coater, followed by performing PB at 130° C. for 60 seconds. Thereafter, cooling was performed at 23° C. for 30 seconds to form a resist film having an average thickness of 55 nm. Next, the resist film was exposed with use of an EUV exposure apparatus (“NXE3300”, manufactured by ASML) with NA of 0.33 under a lighting condition of Conventional s=0.89 and with a mask of imecDEFECT32FFR15. After the exposure, PEB was performed at 120° C. for 60 seconds. Thereafter, the resist film was developed with an organic solvent using n-butyl acetate as an organic solvent developer, and dried to form a negative resist pattern (pattern of contact holes with a 17 nm hole and a 30 nm pitch).

The resist pattern formed using the negative radiation-sensitive composition for EUV exposure was evaluated in the same manner as the resist pattern formed using the negative radiation-sensitive composition for ArF exposure. As a result, the radiation-sensitive composition of Example 80 had good sensitivity, CDU performance, and pattern circularity even when a negative resist pattern was formed by EUV exposure.

According to the radiation-sensitive composition, the method for forming a pattern and the onium salt compound described above, a resist pattern having good sensitivity to exposure light and being superior in LWR performance, pattern rectangularity, CDU performance, pattern circularity, MEEF and

DOF can be formed. Therefore, these can be suitably used for a machining process and the like of a semiconductor device in which micronization is expected to further progress in the future.

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A radiation-sensitive composition comprising:
a first onium salt compound represented by formula (1);
at least one second onium salt compound selected from the group consisting of a carboxylic acid salt compound represented by formula (2) and a carboxylic acid intramolecular salt compound represented by formula (3);
a polymer comprising a structural unit which comprises an acid-dissociable group; and
a solvent, 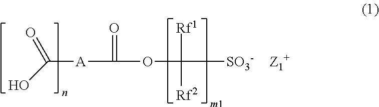
wherein, in the formula (1),
A is a (1+n)-valent organic group having 1 to 40 carbon atoms;
R^{f1} and R^{f2} are each independently a hydrogen atom, a monovalent organic group, a fluorine atom, or a monovalent fluorinated hydrocarbon group; when there are a plurality of R^{f1}s and R^{f2}s, the plurality of R^{f1}s and R^{f2}s are the same as or different from each other;
m1 is an integer of 1 to 8;
n is an integer of 1 to 3; and
Z₁⁺ represents a radiation-sensitive onium cation, 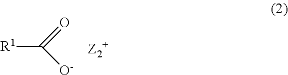
wherein, in the formula (2),
R¹ is a monovalent organic group having 2 to 40 carbon atoms; and
Z₂⁺ represents an organic cation, and 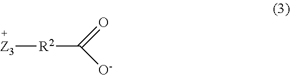
wherein, in the formula (3),
R² is a single bond or a divalent organic group having 1 to 40 carbon atoms; and
Z₃⁺ is a monovalent organic onium cation.

2. The radiation-sensitive composition according to claim 1, wherein in the formula (1), the (1+n)-valent organic group represented by A comprises a first cyclic structure.

3. The radiation-sensitive composition according to claim 2, wherein the first cyclic structure comprises an alicyclic hydrocarbon structure, a cyclic acetal structure, or a combination thereof.

4. The radiation-sensitive composition according to claim 1, wherein in the formula (1), n is 1.

5. The radiation-sensitive composition according to claim 1, wherein in the formula (1), R^{f1} and R^{f2} bonded to a carbon atom adjacent to a sulfur atom in SO₃⁻ are each independently a fluorine atom or a monovalent fluorinated hydrocarbon group.

6. The radiation-sensitive composition according to claim 1, wherein in the formula (2), the monovalent organic group represented by R¹ comprises at least one structure selected from the group consisting of a second cyclic structure, an ester bond, a structure having an alcoholic hydroxyl group, a carboxy group, and an ether bond.

7. The radiation-sensitive composition according to claim 6, wherein the second cyclic structure comprises an alicyclic hydrocarbon structure, an aromatic hydrocarbon group, or a combination thereof.

8. The radiation-sensitive composition according to claim 1, wherein in the formula (3), the monovalent organic onium cation represented by Z₃⁺ is an iodonium cation.

9. The radiation-sensitive composition according to claim 1, wherein a content of the first onium salt compound in the radiation-sensitive composition is 1 part by mass or more and 60 parts by mass or less with respect to 100 parts by mass of the polymer.

10. The radiation-sensitive composition according to claim 1, wherein a content of the second onium salt compound in the radiation-sensitive composition is 1 part by mass or more and 60 parts by mass or less with respect to 100 parts by mass of the polymer.

11. The radiation-sensitive composition according to claim 1, wherein the structural unit which comprises an acid-dissociable group is represented by formula (4), 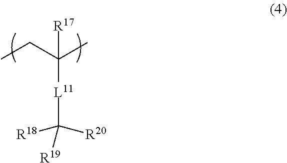
wherein, in the formula (4),
R¹⁷ is a hydrogen atom, a fluorine atom, a methyl group, or a trifluoromethyl group;
R¹⁸ is a substituted or unsubstituted monovalent hydrocarbon group having 1 to 20 carbon atoms;
R¹⁹ and R²⁰ are each independently a substituted or unsubstituted monovalent chain hydrocarbon group having 1 to 20 carbon atoms or a substituted or unsubstituted monovalent alicyclic hydrocarbon group having 3 to 20 carbon atoms, or R¹⁹ and R²⁰ taken together represent a divalent alicyclic group having 3 to 20 carbon atoms together with the carbon atom to which R¹⁹ and R²⁰ are bonded;
L¹¹ represents *—COO—, *-L^{11a}COO—, or *—COOL^{11a}COO—; L^{11a} is a substituted or unsubstituted alkanediyl group or arenediyl group; and
* is a bond to the carbon atom to which R¹⁷ is bonded.

12. A method for forming a pattern, comprising:
applying the radiation-sensitive composition according to claim 1 directly or indirectly to a substrate to form a resist film;
exposing the resist film to light; and
developing the exposed resist film with a developer.

13. The method according to claim 12, wherein exposing comprises exposing the resist film to an ArF excimer laser, extreme ultraviolet rays, or an electron beam.

14. An onium salt compound represented by formula (1-a), 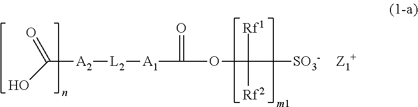
wherein, in the formula (1-a),
A₁ is a divalent organic group;
A₂ is a (1+n)-valent cyclic group;
L₂ is a divalent chain linking group comprising an oxygen atom;
R^{f1} and R^{f2} are each independently a hydrogen atom, a monovalent organic group, a fluorine atom, or a monovalent fluorinated hydrocarbon group; when there are a plurality of R^{f1}s and R^{f2}s, the plurality of R^{f1}s and R^{f2}s are the same as or different from each other;
m1 is an integer of 1 to 8;
n is an integer of 1 to 3; and
Z₁⁺ represents a radiation-sensitive onium cation.

15. The onium salt compound according to claim 14, wherein in the formula (1-a), A₁ is a divalent cyclic group, L₂ is a divalent chain linking group comprising an ester bond or a carbonate bond, and R^{f1} and R^{f2} on a carbon atom at the xx position with respect to a sulfur atom of SO₃⁻ are a fluorine atom or a monovalent fluorinated hydrocarbon group.
